(19) United States
(12) Patent Application Publication
(10) Pub. No.: US 2022/0290194 A1
(43) Pub. Date: Sep. 15, 2022

(54) CANNABIDIOLIC ACID SYNTHASE VARIANTS WITH IMPROVED ACTIVITY FOR USE IN PRODUCTION OF PHYTOCANNABINOIDS

(71) Applicant: Hyasynth Biologicals Inc., Montreal (CA)

(72) Inventors: Letian SONG, Montréal (CA); Timothy S. LIAO, Montréal (CA); Curtis WALTON, Montréal (CA); Louis HOM, Montréal (CA); Mindy MELGAR, Montréal (CA); Daniel FURLONG, Montréal (CA); Devanshi BHARGAVA, Montréal (CA); Sylvester PALYS, Montréal (CA); Leanne BOURGEOIS, Montréal (CA)

(21) Appl. No.: 17/828,449

(22) Filed: May 31, 2022

Related U.S. Application Data
(63) Continuation-in-part of application No. PCT/CA2021/051636, filed on Nov. 18, 2021.
(60) Provisional application No. 63/116,276, filed on Nov. 20, 2020.

(51) Int. Cl. C12P 7/42; C12N 9/02; C12N 9/10; C12N 9/88

(52) U.S. Cl. CPC C12P 7/42; C12Y 121/03008; C12N 9/0004; C12N 9/1029; C12Y 404/01026; C12N 9/88; C12N 9/1085; C12Y 205/01001

(57) ABSTRACT

The present disclosure relates generally to methods, isolated polypeptides and polynucleotides, expression vectors, and host cells for the production of cannabidiolic acid (CBDa), cannabigerolic acid (CBCa), and other phytocannabinoids. A method of producing CBDa, CBCa, and/or other phytocannabinoids in a heterologous host cell having CBDa-producing, CBCa-producing or phytocannabinoid-producing capacity comprises transforming the host cell with a nucleotide encoding a variant CBDa synthase protein having a serine insertion between residues P224 and K225 and one or more other amino acid mutation relative to wild type CBDa synthase, and culturing the transformed host cell to produce CBDa, CBCa, and/or other phytocannabinoids therefrom. The variant CBDa synthase protein has at least 85% sequence identity with the wild type CBDa synthase protein sequence OXC52 according to SEQ ID NO:140, with serine insertion (SEQ ID NO:141). Exemplary variants having good phytocannabinoid production capacity are described.

### CROSS REFERENCE TO RELATED APPLICATIONS

This application is the U.S. National Phase of International Patent Application No. PCT/CA2021/051636 filed Nov. 18, 2021 and is a Continuation-In-Part thereof. This application claims the benefit of and priority to International Patent Application No. PCT/CA2021/051636 filed Nov. 18, 2021, and U.S. Provisional Patent Application No. 63/116,276 filed Nov. 20, 2020, the contents of which are hereby incorporated by reference.

### FIELD

The present disclosure relates generally to proteins having cannabidiolic acid (CBDa) synthase activity, useful in production of phytocannabinoids.

### BACKGROUND

Phytocannabinoids are a large class of compounds with over 100 different known structures that are produced in the Cannabis sativa plant. Phytocannabinoids are known to be biosynthesized in C. sativa, or may result from thermal or other decomposition from phytocannabinoids biosynthesized in C. sativa. These bio-active molecules, such as tetrahydrocannabinol (THC), cannabidiol (CBD), and cannabichromene (CBC) can be extracted from plant material for medical and recreational purposes. However, the synthesis of plant material is costly, not readily scalable to large volumes, and requires lengthy growing periods to produce sufficient quantities of phytocannabinoids. While the C. sativa plant is also a valuable source of grain, fiber, and other material, growing C. sativa for phytocannabinoid production, particularly indoors, is costly in terms of energy and labour. Subsequent extraction, purification, and fractionation of phytocannabinoids from the C. sativa plant is also labour and energy intensive.

Phytocannabinoids are pharmacologically active molecules that contribute to the medical and psychotropic effects of C. sativa. Biosynthesis of phytocannabinoids in the C. sativa plant scales similarly to other agricultural projects. As with other agricultural projects, large scale production of phytocannabinoids by growing C. sativa requires a variety of inputs (e.g. nutrients, light, pest control, CO, etc.). The inputs required for cultivating C. sativa must be provided. In addition, cultivation of C. sativa, where allowed, is currently subject to heavy regulation, taxation, and rigorous quality control where products prepared from the plant are for commercial use, further increasing costs.

Phytocannabinoid analogues are pharmacologically active molecules that are structurally similar to phytocannabinoids. Phytocannabinoid analogues are often synthesized chemically, which can be labour intensive and costly. As a result, it may be economical to produce the phytocannabinoids and phytocannabinoid analogues in a robust and scalable, fermentable organism. Saccharomyces cerevisiae is an example of a fermentable organism that has been used to produce industrial scales of similar molecules.

The extensive time, energy, and labour involved in growing C. sativa for production of naturally-occurring phytocannabinoids provides a motivation to produce phytocannabinoids by other means such as through heterologous pathways in transgenic cell lines. Biosynthesis of phytocannabinoids in C. sativa can include those formed from cannabigerolic acid (CBGa). For example, CBGa may be oxidatively cyclized into cannabidiolic acid (CBDa) by CBDa synthase (Taura et al., 1996).

In addition, it is desirable to find alternative enzymes and methods for the production of phytocannabinoids, and/or for the production of compounds useful in phytocannabinoid biosynthesis as intermediate or precursor compounds.

### SUMMARY

Cannabidiolic acid (CBDa) synthase catalyzes the stereoselective oxidative cyclization of the monoterpene moiety in cannabigerolic acid (CBGa), producing cannabidiolic acid (CBDa). As referenced herein, wild type CBDa synthase (or “OXC52”), can be modified with the insertion of a serine between positions 224 and 225 in the OXC52 sequence, thereby creating a new protein (hereby referred to interchangeably as “OXC154”) with significantly improved CBDa production as compared with OXC52. OXC154 is described in Applicant's co-pending application PCT/CA2020/050687, which is herein incorporated by reference. Variants of OXC154 are described herein that have increased CBDa synthase activity and/or decreased tetrahydrocannabinolic acid (THCa) synthase activity. Exemplary variants are produced in a host cell, showing improved CBDa and/or reduced THCa production. The described variants are useful in the production of cannabidiolic acid and downstream phytocannabinoids in a heterologous host. Methods of production are described.

In certain aspects described, OXC154 variants comprise at least one non-conservative substitution amino acid mutation relative to unmodified OXC154. Certain variants described have improved CBDa synthase activity in comparison to OXC52 and/or OXC154.

A method is described herein for producing cannabidiolic acid (CBDa) cannabichromenic acid (CBCa), or another phytocannabinoid produced therefrom in a heterologous host cell having CBDa-producing, CBCa-producing, or other phytocannabinoid-producing capacity. The method comprises transforming the host cell with a nucleotide encoding a variant cannabidiolic acid (CBDa) synthase protein having a serine insertion between P224 and K225 and one or more other amino acid mutation relative to the wild type CBDa synthase protein OXC52 (SEQ ID NO:140), and culturing the transformed host cell to produce CBDa, CBCa, and/or another phytocannabinoid therefrom, wherein the variant CBDa synthase protein comprises at least 85%, 90%, 95%, or 99% sequence identity with the wild type CBDa synthase protein sequence.

An isolated polypeptide having cannabidiolic acid synthase activity is described, which has an amino acid sequence according to SEQ ID NO:207, wherein 1 or more amino acid residues comprise mutations relative to OXC154 (SEQ ID NO:141). The one or more mutation is located at a position selected from the group consisting of: residues 2, 3, 5, 18, 21, 26, 28, 31, 47, 49, 60, 88, 97, 225, 274, 295, 331, 347, 349, 351, 367, 372, 383, 399, 451, 513, or 515 of SEQ ID NO:141, such as at least at residue 451.

An isolated polynucleotide is described, comprising (a) a nucleotide sequence according to SEQ ID NO:4-SEQ ID NO:71; SEQ ID NO:157-160, SEQ ID NO:165-172, or SEQ ID NO:181-188, SEQ ID NO: 209-210 such as for example SEQ ID NO:187; (b) a nucleotide sequence having at least 85%, at least 90%, at least 95%, at least 99%, or of 100% identity with the nucleotide sequence of (a); or (c) a nucleotide sequence that hybridizes with the complementary strand of the nucleotide having the sequence of (a).

Expression vectors comprising the polynucleotide, and host cells transformed with such expression vectors are described.

Other aspects and features of the present disclosure will become apparent to those ordinarily skilled in the art upon review of the following description of specific embodiments in conjunction with the accompanying figures.

### BRIEF DESCRIPTION OF THE FIGURES

Embodiments of the present disclosure will now be described, by way of example only, with reference to the attached Figures.

FIG. 1 illustrates a cannabinoid biosynthesis pathway in Cannabis sativa.

FIG. 2 illustrates a cannabinoid biosynthesis pathway as described in Applicant's co-pending International Application No: PCT/CA2020/050687.

FIG. 3 illustrates PCR primers used in site-saturation mutagenesis protocol.

FIG. 4 shows stagger-arrayed mutagenic oligonucleotides for combinatorial library construction. The symbol x represents a point mutation.

FIG. 5 shows CBDa production in OXC154 variants.

FIG. 6 shows CBDa production in OXC161 variants in Example 2.

FIG. 7 shows CBDa production values in Example 3.

FIG. 8 shows CBDa production in strains expressing OXC158 variants identified through a combinatorial library in Example 4.

FIG. 9 shows the cannabivarinic acid biosynthesis pathway in Cannabis sativa.

FIG. 10 shows UV spectra of varinoid standards in Example 5.

FIG. 11 shows UV spectra for CBGVa control strain (HB3292, no oxidocyclase).

FIG. 12 shows UV spectra CBDVa strain (HB3291).

FIG. 13 shows CBDVa and intermediate products in strains expressing OXC154 variants identified through a combinatorial library.

FIG. 14A shows panels A to D illustrating production of meroterpenoids in Example 6 in which HB3167 show red fluorescent protein control (RFP).

FIG. 14B shows panels E to H illustrating production of meroterpenoids in Example 6 in which HB3167 show red fluorescent protein control production of meroterpenoids in HB3167 transformed with OXC157.

### DETAILED DESCRIPTION

A method is described for producing cannabidiolic acid (CBDa), cannabichromenic acid, or another phytocannabinoid produced therefrom in a heterologous host cell having CBDa-producing, CBCa-producing, or phytocannabinoid-producing capacity. The method comprises transforming the host cell with a nucleotide encoding a variant cannabidiolic acid (CBDa) synthase protein having a serine insertion between residues P224 and K225, as well as one or more other amino acid mutation relative to the wild type CBDa synthase protein OXC52 (SEQ ID NO:140). The transformed host cell is cultured to produce CBDa, CBCa, and/or a phytocannabinoid therefrom, wherein the variant CBDa synthase protein (referenced interchangeably herein as the OXC154 variant) comprises at least 85%, 90%, 95%, or 99% sequence identity with the wild type CBDa synthase protein sequence.

The one or more other amino acid mutation, aside from the serine insertion that is S225 in OXC154, is at a location selected from the group consisting of: residues 451, 2, 3, 5, 18, 21, 26, 28, 31, 47, 49, 60, 88, 97, 225, 274, 295, 331, 347, 349, 351, 367, 372, 383, 399, 513, and/or 515 OXC154 (SEQ ID NO:141), for example, at least at residue 451. The one or more other mutation may be a conservative or a non-conservative amino acid substitution, and in an exemplary embodiment is a non-conservative substitution. The variant CBDa synthase protein may have a non-conservative amino acid substitution in 2 or more of the noted residues. Optionally, the OXC154 variant protein may additionally have one or more amino acid mutation at a location other than the specified residues (2, 3, 5, 18, 21, 26, 28, 31, 47, 49, 60, 88, 97, 225, 274, 295, 331, 347, 349, 351, 367, 372, 383, 399, 451, 513, or 515 of SEQ ID NO:141) in which the mutation is a conservative amino acid substitution, provided at least 85%, 90%, 95% or 99% sequence identity is maintained, and CBDa synthase activity relative to wild type (OXC52) is maintained.

In an embodiment of the described method, the nucleotide encoding the variant CBDa synthase protein may have a sequence comprising: (a) a nucleotide sequence according to SEQ ID NO:4-SEQ ID NO:71, SEQ ID NO:157-160, SEQ ID NO:165-172, SEQ ID NO:181-188, or SEQ ID NO: 209-210; (b) a nucleotide sequence having at least 85%, 90%, 95% or 99% identity with the sequence of (a); or (c) a nucleotide sequence that hybridizes with the complementary strand of the nucleotide having the sequence of (a), for example, SEQ ID NO:187.

Further, in certain embodiments, the variant CBDa synthase protein may comprise a sequence selected from the group consisting of SEQ ID NO:72 to SEQ ID NO:139, SEQ ID NO:161-164, SEQ ID NO:173-180, or SEQ ID NO:189-196, SEQ ID NO:211, or a sequence of at least 85%, 90%, 95%, or 99% identity thereto, for example, SEQ ID NO:195.

In exemplary embodiments, at least 1 of the one or more other amino acid or codon mutations relative to the wild type CBDa synthase protein OXC52 (SEQ ID NO:140) may be mutations selected from the group consisting of: P2W; R3G, R3T, R3W, R3V, or R3A; NSQ; A18E; L21G; T26A; N28E; L31E; S47F; T49R; S60T; S88A; V97E or V97D; Q274G; N331G; A347G; Q349G; G351I, G351R, or G351M; S367Q; S367N; S367R; or S367K; I372L; A383V; V383A; V383M; V383G; S399G; L451G, P513V; and/or H515E, L451G, based on the residues of OXC154 (SEQ ID NO:141), which mutations are represented by “Xaa” in the variant OXC154 of SEQ ID NO:207.

In the described method, the host cell may be transformed with a nucleotide encoding: (a) a variant CBDa synthase protein with at least 85%, at least 90%, at least 95%, at least 99%, or 100% sequence identity of any one of the following sequences with the indicated substitutions from OXC154 (SEQ ID NO:141):

OXC154-S88A/L451G (SEQ ID NO:72),

OXC154-R3G/L21G/S60T/S88A (SEQ ID NO:73),

OXC154-R3G/A18E/T49R/S60T/S88A (SEQ ID NO:74),

OXC154-R3T/T49R/S88A (SEQ ID NO:75),

OXC154-R3W/A18E/T49R/S60T/S88A (SEQ ID NO:76),

OXC154-R3V/T49R/S60T/S88A (=GCT) (SEQ ID NO:77),

OXC154-R3V/T49R/S60T/S88A (=GCC) (SEQ ID NO:78),

OXC154-A18A (SEQ ID NO:79),

OXC154-R3T/A18E/T49R/S88A (=GCC) (SEQ ID NO:80),

OXC154-R3T/S88A (=GCC) (SEQ ID NO:81),

OXC154-R3G(=GGG)/L21G/T49R (=GCC) (SEQ ID NO:82),

OXC154-R3T/T49R/S88A(=GCT) (SEQ ID NO:83),

OXC154-R3G(=GGA)/A18E/T49R/S60T/S88A(=GCC) (SEQ ID NO:84),

OXC154-R3W/T49R/S88A(=GCC)/V97E (SEQ ID NO:85),

OXC154-R3G(=GGG)/A18E/S88A(=GCC) (SEQ ID NO:86),

OXC154-R3V/A18E/T49R/S60T/S88A(=GCC) (SEQ ID NO:87),

OXC154-S60T/S88A(=GCC) (SEQ ID NO:88),

OXC154-R3T/A18E/T49R/S60T/S88A(=GCT) (SEQ ID NO:89),

OXC154-R3W/L21G/T49R/S88A(=GCC)/V97E (SEQ ID NO:90),

OXC154-R3T/A18E/T49R/S60T (SEQ ID NO:91),

OXC154-P2W/T26A/S60T (SEQ ID NO:91),

OXC154-R3G(=GGG)/L21G/S60T/S88A(=GCC)/V97E (SEQ ID NO:93),

OXC154-R3G(=GGG)/A18E/T49R/S88A(=GCC) (SEQ ID NO:94),

OXC154-R3T/L21G/S60T/S88A(=GCC)/V97D (SEQ ID NO:95),

OXC154-P2W/L21G/T49R/S88A(=GCC)/V97E (SEQ ID NO:96),

OXC154-R3G(=GGG)/L21G/T49R/S88A(=GCT) (SEQ ID NO:97),

OXC154-S295S(=TCA) (SEQ ID NO:98),

OXC154-R3V/L21G/S60T/S88A(=GCC) (SEQ ID NO:99),

OXC154-R3T/A18E/S88A(=GCC) (SEQ ID NO:100),

OXC154-S60T/S88A(=GCT) (SEQ ID NO:101),

OXC154-R3W/T49R/S88A(=GCT) (SEQ ID NO:102),

OXC154-T49R/S88A(=GCC) (SEQ ID NO:103),

OXC154-R3W/S47F (SEQ ID NO:104),

OXC154-A347G/I372L/L451G (SEQ ID NO:105),

OXC154-R3G(=GGG)/L21G/S60T (SEQ ID NO:106),

OXC154-R3T/L21G/T49R/S88A(=GCT) (SEQ ID NO:107),

OXC154-R3T/L21G/S60T (SEQ ID NO:108),

OXC154-R3W/L21G/S88A(=GCT) (SEQ ID NO:109),

OXC154-L21G/T49R/S60T/S88A(=GCT) (SEQ ID NO:110),

OXC154-A347G/A383V (SEQ ID NO:111),

OXC154-R3W/L21G/T49R/S60T/S88A(=GCT) (SEQ ID NO:112),

OXC154-A18E/S88A(=GCC) (SEQ ID NO:113),

OXC154-R3W/L21G/T49R (SEQ ID NO:114),

OXC154-A347G/L451G (SEQ ID NO:115),

OXC154-A347G/I372L/A383V/L451G (SEQ ID NO:116),

OXC154-I372L/A383V/L451G (SEQ ID NO:117),

OXC154-R3V/T49R/S88A(=GCT) (SEQ ID NO: 118),

OXC154-R3G(=GGG)/A18E/S60T (SEQ ID NO:119),

OXC154-A347G/I372L/A383V (SEQ ID NO:120),

OXC154-R3T (SEQ ID NO:121),

OXC154-R3V/A18E/T49R/V97E (SEQ ID NO:122),

OXC154-R3T/L21G/T49R/S60T/S88A(=GCT) (SEQ ID NO:123),

OXC154-R3T/L21G/T49R/V97E (SEQ ID NO:124),

OXC154-R3V/L21G/T49R/S60T (SEQ ID NO:125),

OXC154-G351I/1372L (SEQ ID NO:126),

OXC154-G351I/A383V/L451G (SEQ ID NO:127),

OXC154-G351R/I372L/L451G (SEQ ID NO:128),

OXC154-G351I/I372L/A383V/L451G (SEQ ID NO:129),

OXC154-G351R/I372L/A383V/L451G (SEQ ID NO:130),

OXC154-G351I/I372L/A383V (SEQ ID NO:131),

OXC154-N331G/Q349G/I372L/L451G (SEQ ID NO:132),

OXC154-G351R/A383V/L451G (SEQ ID NO:133),

OXC154-Q349G/A383V/L451G (SEQ ID NO:134),

OXC154-A383V/L451G (SEQ ID NO:135),

OXC154-N331G/Q349G (SEQ ID NO:136),

OXC154-G351I (SEQ ID NO:137),

OXC154-L451G (SEQ ID NO:138),

OXC154-N331G/G351I/1372L/A383V (SEQ ID NO:139),

OXC154-R3G/A18E/S60T/G351I/A383V/L451G (SEQ ID NO:161),

OXC154-R3W/A18E/T49R/V97E/G351I/A383V/L451G (SEQ ID NO:162),

OXC154-R3W/A18E/T49R/V97E/G351I/A383V/L451G (SEQ ID NO:163),

OXC154-R3T/S60T/G351I/A383V/L451G (SEQ ID NO:164), or

OXC154-R3G/A18E/S60T/G351I/A383V/L451G (SEQ ID NO: 211).

Alternatively, the cell may be transformed with a nucleotide encoding a variant CBDa synthase protein with at least 85%, at least 90%, at least 95%, at least 99% sequence identity, or with 100% identity with any one of the following sequences with the further indicated substitutions from OXC158 (SEQ ID NO:162):

OXC158-W3A/I351G/V383A (SEQ ID NO:195),

OXC158-I351G (SEQ ID NO:173),

OXC158-S367R(=CGG) (SEQ ID NO:174),

OXC158-Q274G (SEQ ID NO:175),

OXC158-I351M (SEQ ID NO:176),

OXC158-V383A (SEQ ID NO:177),

OXC158-S367Q (SEQ ID NO:178),

OXC158-S367N (SEQ ID NO:179),

OXC158-S367R(=AGG) (SEQ ID NO:180),

OXC158-L31E/V383G (SEQ ID NO:189),

OXC158-N138T/V383M/H515E (SEQ ID NO:190),

OXC158-S367K/V383A/P513V (SEQ ID NO:191),

OXC158-V383A (SEQ ID NO:192),

OXC158-W3A/L31E/K226M/S367Q/V383M/S399G/P513V (SEQ ID NO:193),

OXC158-I351GN383A (SEQ ID NO:194), or

OXC158-W3A/N5Q/N28E/I351G/S367R/V383A (SEQ ID NO:196).

Of these, one exemplary sequence is OXC158-W3A/I351G/V383A (SEQ ID NO:195).

In the method, the production of a phytocannabinoid by the transformed host cell may involve production of phytocannabinoids including but not limited to cannabigerol (CBG), cannabigerolic acid (CBGa), cannabigerovarin (CBGv), cannabigerovarinic acid (CBGVa), cannabigerocin (CBGO), cannabigerocinic acid (CBGOa), cannabidiovarinic acid (CBDVa), cannabichromenic acid (CBCa), cannabichromene (CBC), tetrahydrocannabinol (THC), or tetrahydrocannabinolic acid (THCa). For example, the transformed host cell may produce cannabidiovarinic acid (CBDVa) from cannabigerovarinic acid (CBGVa). Further, when the transformed host cell is one that produces cannabidiovarinic acid (CBDVa) from cannabigerovarinic acid (CBGVa), this may be done in the presence of endogenously produced or exogenously provided butyric acid.

The host cell transformed in the method described may be a yeast cell, a bacterial cell, a fungal cell, a protist cell, or a plant cell. Exemplary organisms include S. cerevisiae, E. coli, Yarrowia lipolytica, or Komagataella phaffii, as well as others described herein. The transformed host cell may additionally comprise, or be transformed with, other enzymes useful in phytocannabinoid production. For example, a polynucleotide encoding a polyketide synthase enzyme, a polynucleotide encoding an olivetolic acid cyclase enzyme, and/or a polynucleotide encoding a prenyltransferase enzyme may also be included in the host cell. Further options for polynucleotides and methods, such as described in Applicant's co-pending International Application No: PCT/CA2020/050687 (hereby incorporated by reference) are envisioned. The transformed host cell may comprises a polynucleotide encoding a type III PKS, an acyl-activating enzyme, a prenyltransferase enzyme, and/or an oxidocyclase enzyme.

An isolated polypeptide is described herein, having cannabidiolic acid synthase activity and comprising an amino acid sequence of at least 85%, of at least 90%, of at least 95%, of at least 99%, or of 100% sequence identity relative to OXC154 (SEQ ID NO:141), wherein 1 or more amino acid residues comprise mutations relative to OXC154 (SEQ ID NO:141), at least one of said one or more mutation being located at a position selected from the group consisting of: residues 2, 3, 5, 18, 21, 26, 28, 31, 47, 49, 60, 88, 97, 225, 274, 295, 331, 347, 349, 351, 367, 372, 383, 399, 451, 513, or 515 of SEQ ID NO:141 of SEQ ID NO:141. The isolated polypeptide may comprise an amino acid sequence according to SEQ ID NO:72-SEQ ID NO:139, SEQ ID NO:161-164, SEQ ID NO:173-180, or SEQ ID NO:189-196, for example SEQ ID NO:195.

An isolated polynucleotide is described, comprising: (a) a nucleotide sequence according to SEQ ID NO:4-SEQ ID NO:71, SEQ ID NO:157-160, SEQ ID NO:165-172, or SEQ ID NO:181-188 (b) a nucleotide sequence having at least 85%, 90%, 95%, or 99% identity with the nucleotide sequence of (a), or (c) a nucleotide sequence that hybridizes with the complementary strand of the nucleotide having the sequence of (a).

An expression vector comprising the polynucleotide is described, such that the vector encodes a variant CBDa synthase protein with a sequence as described, with CBDa synthase activity. Such an expression vector encodes the variant CBDa synthase protein by comprising a nucleotide sequence according to any of SEQ ID NO:4 to SEQ ID NO:71; SEQ ID NO:157-160, SEQ ID NO:165-172, or SEQ ID NO:181-188, or having 85%, 90%, 95%, 99% identity to these sequences.

A host cell transformed with the expression vector as described may additionally comprise a polynucleotide encoding a polyketide synthase enzyme, a polynucleotide encoding an olivetolic acid cyclase enzyme, and/or a polynucleotide encoding a prenyltransferase enzyme. Such a host cell may comprise a polynucleotide encoding other enzymes useful in synthesis of olivetolic acid and/or phytocannabinoids. The host cell may comprises a polynucleotide encoding a type III PKS, an acyl-activating enzyme, a prenyltransferase enzyme, and/or an oxidocyclase enzyme. The host cell may be a yeast, a bacterial cell, a fungal cell, a protist cell, or a plant cell, for example: S. cerevisiae, E. coli, Yarrowia lipolytica, or Komagataella phaffii.

### Definitions

Certain terms used herein are described below.

The term “cannabinoid” as used herein refers to a chemical compound that shows direct or indirect activity at a cannabinoid receptor. Non limiting examples of cannabinoids include tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM).

The term “phytocannabinoid” as used herein refers to a cannabinoid that typically occurs in a plant species. Exemplary phytocannabinoids produced according to the invention include cannabigerol (CBG); cannabigerolic acid (CBGa); cannabivarins such as cannabigerovarin (CBGV), cannabigerovarinic acid (CBGVa), or cannabidiovarinic acid (CBDVa); cannabigerocin (CBGo); or cannabigerocinic acid (CBGoa).

Cannabinoids and phytocannabinoids may contain or may lack one or more carboxylic acid functional groups. Non limiting examples of such cannabinoids or phytocannabinoids containing carboxylic acid function groups or phytocannabinoids include tetrahydrocannabinolic acid (THCA), cannabidiolic acid (CBDA), and cannabichromenic acid (CBCA).

The term “homologue” includes homologous sequences from the same and other species and orthologous sequences from the same and other species. Different polynucleotides or polypeptides having homology may be referred to as homologues.

The term “homology” may refer to the level of similarity between two or more polynucleotide and/or polypeptide sequences in terms of percent of positional identity (i.e., sequence similarity or identity). Homology also refers to the concept of similar functional properties among different polynucleotide or polypeptides. Thus, the compositions and methods herein may further comprise homologues to the polypeptide and polynucleotide sequences described herein.

The term “orthologous,” as used herein, refers to homologous polypeptide sequences and/or polynucleotide sequences in different species that arose from a common ancestral gene during speciation.

As used herein, a “homologue” may have a significant sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% and/or 100%) to the polynucleotide sequences herein.

As used herein “sequence identity” refers to the extent to which two optimally aligned polynucleotide or peptide sequences are invariant throughout a window of alignment of components, e.g., nucleotides or amino acids. “Identity” can be readily calculated by known methods.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

The terms “fatty acid-CoA”, “fatty acyl-CoA”, or “CoA donors” as used herein may refer to compounds useful in polyketide synthesis as primer molecules which react in a condensation reaction with an extender unit (such as malonyl-CoA) to form a polyketide. Examples of fatty acid-CoA molecules (also referred to herein as primer molecules or CoA donors), useful in the synthetic routes described herein include but are not limited to: acetyl-CoA, butyryl-CoA, hexanoyl-CoA. These fatty acid-CoA molecules may be provided to host cells or may be synthesized by the host cells for biosynthesis of polyketides, as described herein.

Two nucleotide sequences can be considered to be substantially “complementary” when the two sequences hybridize to each other under stringent conditions. In some examples, two nucleotide sequences considered to be substantially complementary hybridize to each other under highly stringent conditions.

The terms “stringent hybridization conditions” and “stringent hybridization wash conditions” in the context of nucleic acid hybridization experiments, for example in Southern hybridizations and Northern hybridizations are sequence dependent, and are different under different environmental parameters. In some examples, generally, highly stringent hybridization and wash conditions are selected to be about 5° C. lower than the thermal melting point (Tm) for the specific sequence at a defined ionic strength and pH.

In some examples, polynucleotides include polynucleotides or “variants” having at least about 50%, 55%, 60%, 65%, 70%, 75%, 80%, 85%, 90%, 95%, 96%, 97%, 98%, 99% or 100% sequence identity to any of the reference sequences described herein, typically where the variant maintains at least one biological activity of the reference sequence.

As used herein, the terms “polynucleotide variant” and “variant” and the like refer to polynucleotides displaying substantial sequence identity with a reference polynucleotide sequence or polynucleotides that hybridize with a reference sequence under, for example, stringent conditions. These terms may include polynucleotides in which one or more nucleotides have been added or deleted, or replaced with different nucleotides compared to a reference polynucleotide. It will be understood that certain alterations inclusive of mutations, additions, deletions and substitutions can be made to a reference polynucleotide whereby the altered polynucleotide retains the biological function or activity of the reference polynucleotide.

In some examples, the polynucleotides described herein may be included within “vectors” and/or “expression cassettes”.

In some embodiments, the nucleotide sequences and/or nucleic acid molecules described herein may be “operably” or “operatively” linked to a variety of promoters for expression in host cells. Thus, in some examples, the invention provides transformed host cells and transformed organisms comprising the transformed host cells, wherein the host cells and organisms are transformed with one or more nucleic acid molecules/nucleotide sequences of the invention. As used herein, “operably linked to,” when referring to a first nucleic acid sequence that is operably linked to a second nucleic acid sequence, means a situation when the first nucleic acid sequence is placed in a functional relationship with the second nucleic acid sequence. For instance, a promoter is operably associated with a coding sequence if the promoter effects the transcription or expression of the coding sequence.

In the context of a polypeptide, “operably linked to,” when referring to a first polypeptide sequence that is operably linked to a second polypeptide sequence, refers to a situation when the first polypeptide sequence is placed in a functional relationship with the second polypeptide sequence.

The term “promoter,” as used herein, refers to a nucleotide sequence that controls or regulates the transcription of a nucleotide sequence (i.e., a coding sequence) that is operably associated with the promoter. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression.

Promoters can include, for example, constitutive, inducible, temporally regulated, developmentally regulated, chemically regulated, tissue-preferred and tissue-specific promoters for use in the preparation of recombinant nucleic acid molecules, i.e., chimeric genes.

The choice of promoter will vary depending on the temporal and spatial requirements for expression, and also depending on the host cell to be transformed. Thus, for example, where expression in response to a stimulus is desired a promoter inducible by stimuli or chemicals can be used. Where continuous expression at a relatively constant level is desired throughout the cells or tissues of an organism a constitutive promoter can be chosen.

In some examples, vectors may be used.

In some examples, the polynucleotide molecules and nucleotide sequences described herein can be used in connection with vectors.

The term “vector” refers to a composition for transferring, delivering or introducing a nucleic acid or polynucleotide into a host cell. A vector may comprise a polynucleotide molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid, a fosmid, a bacteriophage, or an artificial chromosome. The selection of a vector will depend upon the preferred transformation technique and the target species for transformation.

As used herein, “expression vectors” refers to a nucleic acid molecule comprising a nucleotide sequence of interest, wherein said nucleotide sequence is operatively associated with at least a control sequence (e.g., a promoter). Thus, some examples provide expression vectors designed to express the polynucleotide sequences described herein.

An expression vector comprising a polynucleotide sequence of interest may be “chimeric”, meaning that at least one of its components is heterologous with respect to at least one of its other components. An expression cassette may also be one that is naturally occurring but has been obtained in a recombinant form useful for heterologous expression. In some examples, however, the expression vector is heterologous with respect to the host. For example, the particular polynucleotide sequence of the expression vector does not occur naturally in the host cell and must have been introduced into the host cell or an ancestor of the host cell by a transformation event.

In some examples, an expression vector may also include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, promoters, enhancers, introns, 5′ and 3′ untranslated regions, translation leader sequences, termination signals, and polyadenylation signal sequences.

An expression vector may also include a nucleotide sequence for a selectable marker, which can be used to select a transformed host cell.

As used herein, “selectable marker” means a nucleotide sequence that when expressed imparts a distinct phenotype to the host cell expressing the marker and thus allows such transformed host cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, a sugar, a carbon source, or the like), or on whether the marker is simply a trait that one can identify through observation or testing, such as by screening. Examples of suitable selectable markers are known in the art and can be used in the expression vectors described herein.

The vector and/or expression vectors and/or polynucleotides may be introduced into a cell.

The term “introducing,” in the context of a nucleotide sequence of interest (e.g., the nucleic acid molecules/constructs/expression vectors), refers to presenting the nucleotide sequence of interest to cell host in such a manner that the nucleotide sequence gains access to the interior of a cell. Where more than one nucleotide sequence is to be introduced these nucleotide sequences can be assembled as part of a single polynucleotide or nucleic acid construct, or as separate polynucleotide or nucleic acid constructs, and can be located on the same or different transformation vectors. Accordingly, these polynucleotides may be introduced into host cells in a single transformation event, or in separate transformation events.

As used herein, the term “contacting” refers to a process by which, for example, a compound may be delivered to a cell. The compound may be administered in a number of ways, including, but not limited to, direct introduction into a cell (i.e., intracellularly) and/or extracellular introduction into a cavity, interstitial space, or into the circulation of the organism.

The term “transformation” or “transfection” as used herein refers to the introduction of a polynucleotide or heterologous nucleic acid into a cell. Transformation of a cell may be stable or transient.

The term “transient transformation” as used herein in the context of a polynucleotide refers to a polynucleotide introduced into the cell and does not integrate into the genome of the cell.

The terms “stably introducing” or “stably introduced” in the context of a polynucleotide introduced into a cell is intended to represent that the introduced polynucleotide is stably incorporated into the genome of the cell, and thus the cell is stably transformed with the polynucleotide.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transformed in vivo or in vitro with a recombinant vector or a polynucleotide of the invention. A host cell which comprises a recombinant vector of the invention is a recombinant host cell.

In some examples, a host cell may be a bacterial cell, a fungal cell, a protist cell, or a plant cell. Specific examples of host cells are described below.

“Conversion” refers to the enzymatic transformation of a substrate to the corresponding product. “Percent conversion” refers to the percent of the substrate that is converted to the product within a period of time under specified conditions. Thus, for example, the “activity” or “conversion rate” of a ketoreductase polypeptide can be expressed as “percent conversion” of the substrate to the product.

“Hydrophilic Amino Acid or Residue” refers to an amino acid or residue having a side chain exhibiting a hydrophobicity of less than zero according to the normalized consensus hydrophobicity scale Eisenberg et al., 1984. Genetically encoded hydrophilic amino acids include L-Thr (T), L-Ser (S), L-His (H), L-Glu (E), L-Asn (N), L-Gln (Q), L-Asp (D), L-Lys (K) and L-Arg (R).

“Acidic Amino Acid or Residue” refers to a hydrophilic amino acid or residue having a side chain exhibiting a pKa value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic Amino Acid or Residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ion. Genetically encoded basic amino acids include L-Arg (R) and L-Lys (K).

“Polar Amino Acid or Residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S) and L-Thr (T).

“Hydrophobic Amino Acid or Residue” refers to an amino acid or residue having a side chain exhibiting a hydrophobicity of greater than zero according to the normalized consensus hydrophobicity scale (Eisenberg et al., 1984). Genetically encoded hydrophobic amino acids include L-Pro (P), L-Ile (I), L-Phe (F), L-Val (V), L-Leu (L), L-Trp (W), L-Met (M), L-Ala (A) and L-Tyr (Y).

“Aromatic Amino Acid or Residue” refers to a hydrophilic or hydrophobic amino acid or residue having a side chain that includes at least one aromatic or heteroaromatic ring. Genetically encoded aromatic amino acids include L-Phe (F), L-Tyr (Y) and L-Trp (W). Although owing to the pKa of its heteroaromatic nitrogen atom L His (H) it is sometimes classified as a basic residue, or as an aromatic residue as its side chain includes a heteroaromatic ring, herein histidine is classified as a hydrophilic residue.

“Constrained amino acid or residue” refers to an amino acid or residue that has a constrained geometry. Herein, constrained residues include L-Pro (P) and L-His (H). Histidine has a constrained geometry because it has a relatively small imidazole ring. Proline has a constrained geometry because it also has a five membered ring.

“Non-polar Amino Acid or Residue” refers to a hydrophobic amino acid or residue having a side chain that is uncharged at physiological pH and which has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M) and L-Ala (A).

“Aliphatic Amino Acid or Residue” refers to a hydrophobic amino acid or residue having an aliphatic hydrocarbon side chain. Genetically encoded aliphatic amino acids include L-Ala (A), L-Val (V), L-Leu (L) and L-Ile (I).

“Small Amino Acid or Residue” refers to an amino acid or residue having a side chain that is composed of a total three or fewer carbon and/or heteroatoms (excluding the α-carbon and hydrogens). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser (S), L-Thr (T) and L-Asp (D).

A “conservative” amino acid substitution (or mutation) refers to the substitution of a residue with a residue having a similar side chain, and thus typically involves substitution of the amino acid in the polypeptide with amino acids within the same or similar defined class of amino acids. For the following residues, the possible conservative mutations are provided in parentheses: A, L, V, I (Other aliphatic residues: A, L, V, 1); A, L, V, I, G, M (Other non-polar residues: A, L, V, I, G, M); D, E (Other acidic residues: D, E); K, R (Other basic residues: K, R); P, H (Other constrained residues: P, H); N, Q, S, T (Other polar residues: N, Q, S, T); Y, W, F (Other aromatic residues: Y, W, F); and C (none).

Phytocannabinoids are a large class of compounds with over 100 different known structures that are produced in the Cannabis plant. These bio-active molecules, such as tetrahydrocannabinol (THC) and cannabidiol (CBD), can be extracted from plant material for medical and psychotropic purposes. However, the synthesis of plant material is costly, not readily scalable to large volumes, and requires lengthy growth periods to produce sufficient quantities of phytocannabinoids. A fermentable organism such as Saccharomyces cerevisiae capable of producing cannabinoids would provide an economical route to producing these compounds on an industrial scale. The extensive time, energy, and labour involved in growing C. sativa for phytocannabinoid production provides a motivation to produce transgenic cell lines for production of phytocannabinoids in yeast. One example of such efforts is provided in PCT application by Mookerjee et al WO2018/148848.

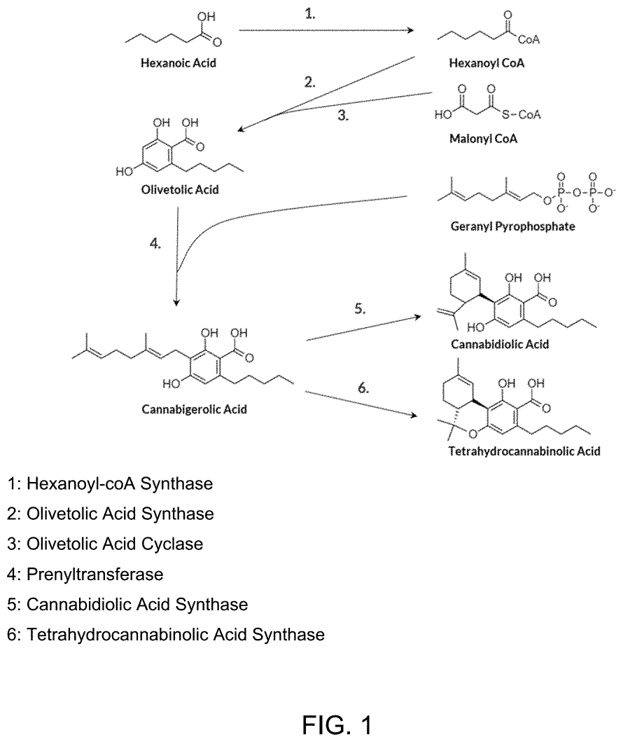

FIG. 1 illustrates a cannabinoid biosynthesis pathway in Cannabis sativa. As expression and functionality of the C. sativa pathway in S. cerevisiae is hindered by problems of toxic precursors and poor expression, a novel biosynthetic route for cannabinoid production was developed that overcomes said issues.

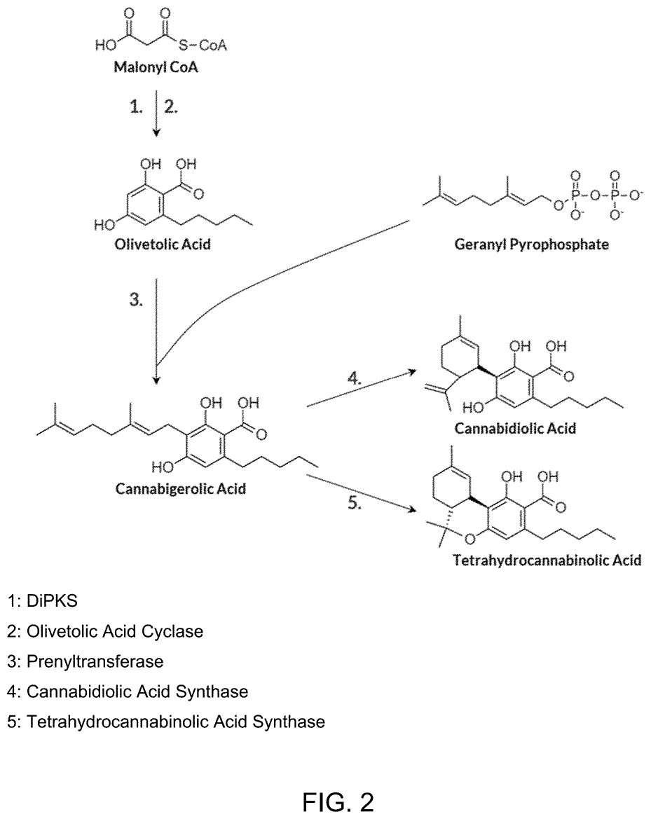

The pathway is described in FIG. 2 comprises a multi-enzyme system. DiPKS from D. discoideum and OAC from C. sativa are used to produce olivetolic acid directly from glucose. GPP from the yeast terpenoid pathway and OLA are subsequently converted to cannabigerolic acid catalyzed by using a prenyltransferase. Then, C. sativa THCa synthase or CBDa synthase is used to further cyclize cannabigerolic acid to form THCa or CBDa respectively.

FIG. 2 illustrates a cannabinoid biosynthesis pathway as described in Applicant's co-pending PCT Application No. CA2020/050687 (Bourgeois et al., filed May 21, 2019), which is herein incorporated by reference.

The biosynthesis of downstream acid forms of phytocannabinoids in C. sativa from cannabigerolic acid (CBGa) is illustrated in steps 4 and 5 of FIG. 2. CBGa is oxidatively cyclized into A9-tetrahydrocannabinolic acid (THCa) by THCa synthase. As depicted herein, CBGa is oxidatively cyclized into cannabidiolic acid (CBDa) by CBDa synthase. PCT Application No: CA2020/050687 describes modified CBDa synthases, for example those referred to as Ostl-pro-alpha-f(I)-OXC52 and mutants thereof.

One mutant described in Applicant's co-pending PCT Application No. CA2020/050687 is referenced from strain HB2010, which is a mutant of OXC52 with a serine insertion between residues P224 and K225. Another mutant is from strain HB1973; a mutant of OXC52 having mutations S88A, L450G, and a serine insertion between residues 224 and 225, the sequence of which is provided in Applicant's co-pending International Patent Application PCT/CA2020/050687 and is hereby incorporated by reference. The protein is described as having the general description “Ostl-pro-alpha-f(I)-OXC52-Serine insertion between residues 224 and 225” is herein referred to interchangeably as “Ostl-pro-alpha-f(I)-OXC154”. Other variants pertaining to OXC52 are described in PCT Application No. CA2020/050687 such as variants referred to as “OXC155” and “OXC53”.

The term “CBDa synthase” refers to an oxidoreductase that converts CBGa into CBDa by stereo-selectively cyclizing the monoterpene moiety in CBGa, as shown in step 5 of FIG. 1. Wild type CBDa synthase isolated from Cannabis sativa (referred to herein as OXC52) has a protein sequence of 523 amino acids (or variants with 544 amino acids including an N-terminal signal peptide of 28 amino acids (Uniprot ID: A6P6V9). As referred to herein, the wild type CBDa synthase is encoded by the DNA sequence of SEQ ID NO:1. In yeast, proper CBDa synthase functionality requires localization to the vacuole. As described herein, when expressing CBDa synthases the native N-terminal signal peptide is removed from the enzyme and is replaced with an N-terminal Ostl-pro-alpha-f(I) tag (SEQ ID NO:156). All oxidocyclase sequences listed in this application have an added 3′-terminal 6 amino acid histidine tag (SEQ ID NO:206) to assist in protein purification where necessary.

CBDa synthase predominantly utilizes cannabigerolic acid (CBGa) as substrate to form CBDa, and also accepts cannabinerolic acid, an isomer of CBGa, with low catalytic activity. The oxidocyclization reaction catalyzed by CBDa synthase requires the FAD coenzyme but does not require molecular oxygen or other metal ion cofactors (Taura et al., 1996). The main reaction product is CBDa accompanied with a small amount of THCa and CBCa by-products.

A modified CBDa synthase is described herein that has a serine inserted between residues P224 and K225 of the wild type sequence and is hereafter referred to as OXC154 (encoded by a nucleotide according to SEQ ID NO:2), the amino acid sequence of which is provided as SEQ ID NO:141. In order to further improve the activity and product specificity of Ostl-pro-alpha-f(I)-OXC154 inside yeast, protein engineering was conducted on OXC154. Numerous variants were identified from the process displaying increased CBDa synthase activity and/or decreased THCa synthase activity. Sixty-eight such variants are exemplified herein. The variants described have at least one point mutation relative to the amino acid sequence of OXC154. The amino acid sequence illustrating candidate positions for modified residue locations is provided as SEQ ID NO:207.

The process of producing CBDa in a modified yeast cell using these enzymes is described herein.

Enzyme engineering is the process of improving a desired phenotype of the enzyme by making modifications to the amino acid sequence of the polypeptide. As the functionality of the enzyme is dependent on the structure of the enzyme and the structure of the enzyme is dependent, partially, on the primary amino acid sequence; modification of the amino acid sequence of the enzyme can lead to a beneficial impact on the desired phenotype. This principle was applied to OXC154, as described herein, and modifications were made to its amino acid sequence using a directed evolution approach, allowing identification of amino acid residues that improved activity in a strain of recombinant S. cerevisiae.

Sequences are described herein that have multiple residues modified as compared to the OXC154 sequence, which modifications allow the variant enzyme to catalyze the production of CBDa with greater demonstrated product conversion as compared to the OXC154. In some instances, improved product conversion may range up to 300% greater, for example more than 245% greater, more than 200% greater. Other levels of improvement are observed in different variants. Improvements to one or more enzyme properties of the engineered OXC154 variants may include increases in enzyme activity, enzyme kinetics and turnover, tolerance to increased levels of substrate, and tolerance to increased product levels. The modifications of the residues may be conservative modifications/substitutions or non-conservative modifications/substitutions.

According to embodiments described herein, the residues that can be modified will be defined as X{#} where # represents the sequence position in the amino acid position of the wild type OXC154 sequence (SEQ ID NO:2). Specifically the following 17 residues may be modified in the OXC154 variants according to SEQ ID NO:207: X{2}, X{3}, X{18}, X{21}, X{26}, X{47}, X{49}, X{60}, X{88}, X{97}, X{225}, X{295}, X{331}, X{347}, X{349}, X{351}, X{372}, X{383}, X{451}, among others.

Further, the following additional residues may be modified in this sequence: X{5}, X{28}, X{31}, X{274}, X{367}, X{399}, X{513}, X{515}.

SEQ ID NO:140 represents the wild type cannabidiolic acid (CBDa) synthase protein OXC52:

| | |
|---|---|
| MPRENFLKCF SQYIPNNATN LKLVYTQNNP LYMSVLNSTI HNLRFTSDTT | 50 |
| PKPLVIVTPS HVSHIQGTIL CSKKVGLQIR TRSGGHDSEG MSYISQVPFV | 100 |
| IVDLRNMRSI KIDVHSQTAW VEAGATLGEV YYWVNEKNEN LSLAAGYCPT | 150 |
| VCAGGHFGGG GYGPLMRNYG LAADNIIDAH LVNVHGKVLD RKSMGEDLFW | 200 |
| ALRGGGAESF GIIVAWKIRL VAVPKSTMFS VKKIMEIHEL VKLVNKWQNI | 250 |
| AYKYDKDLLL MTHFITRNIT DNQGKNKTAI HTYFSSVFLG GVDSLVDLMN | 300 |
| KSFPELGIKK TDCRQLSWID TIIFYSGVVN YDTDNFNKEI LLDRSAGQNG | 350 |
| AFKIKLDYVK KPIPESVFVQ ILEKLYEEDI GAGMYALYPY GGIMDEISES | 400 |
| AIPFPHRAGI LYELWYICSW EKQEDNEKHL NWIRNIYNFM TPYVSKNPRL | 450 |
| AYLNYRDLDI GINDPKNPNN YTQARIWGEK YFGKNFDRLV KVKTLVDPNN | 500 |
| FFRNEQSIPP LPRHRHGHHH HHH | 523 |

SEQ ID NO:141 represents the modified cannabidiolic acid (CBDa) synthase protein OXC154, which differs from OXC52 by having a serine S insertion between residues P224 and K225 relative to OXC52 (SEQ ID NO:140):

| | |
|---|---|
| MPRENFLKCF SQYIPNNATN LKLVYTQNNP LYMSVLNSTI HNLRFTSDTT | 50 |
| PKPLVIVTPS HVSHIQGTIL CSKKVGLQIR TRSGGHDSEG MSYISQVPFV | 100 |
| IVDLRNMRSI KIDVHSQTAW VEAGATLGEV YYWVNEKNEN LSLAAGYCPT | 150 |
| VCAGGHFGGG GYGPLMRNYG LAADNIIDAH LVNVHGKVLD RKSMGEDLFW | 200 |
| ALRGGGAESF GIIVAWKIRL VAVPSKSTMF SVKKIMEIHE LVKLVNKWQN | 250 |
| IAYKYDKDLL LMTHFITRNI TDNQGKNKTA IHTYFSSVFL GGVDSLVDLM | 300 |
| NKSFPELGIK KTDCRQLSWI DTIIFYSGVV NYDTDNFNKE ILLDRSAGQN | 350 |
| GAFKIKLDYV KKPIPESVFV QILEKLYEED IGAGMYALYP YGGIMDEISE | 400 |
| SAIPFPHRAG ILYELWYICS WEKQEDNEKHL NWIRNIYNF MTPYVSKNPR | 450 |
| LAYLNYRDLD IGINDPKNPN NYTQARIWGE KYFGKNFDRL VKVKTLVDPN | 500 |
| NFFRNEQSIP PLPRHRHGHH HHHH | 524 |

SEQ ID NO:207 represents the generalized variant CBDa synthase protein OXC154 of SEQ ID NO:141 (including the serine S insertion that is S225), but with candidate locations for mutated residues represented as X (where X represents any amino acid):

| | |
|---|---|
| MXXEXFLKCF SQYIPNNXTN XKLVYXQXNP XYMSVLNSTI HNLRFTXDXT | 50 |
| PKPLVIVTPX HVSHIQGTIL CSKKVGLQIR TRSGGHDXEG MSYISQXPFV | 100 |
| IVDLRNMRSI KIDVHSQTAW VEAGATLGEV YYWVNEKNEN LSLAAGYCPT | 150 |
| VCAGGHFGGG GYGPLMRNYG LAADNIIDAH LVNVHGKVLD RKSMGEDLFW | 200 |
| ALRGGGAESF GIIVAWKIRL VAVPXKSTMF SVKKIMEIHE LVKLVNXWQN | 250 |
| IAYKYDKDLL LMTHFITRNI TDNQGKNKTA IHTYFSSVFL GGVDSLVDLM | 300 |
| NKSFPELGIK KTDCRQLSWI DTIIFYSGVV XYDTDNFNKE ILLDRSXGXN | 350 |
| XAFKIKLDYV KKPIPEXVFV QXLEKLYEED IGXGMYALYP YGGIMDEIXE | 400 |
| SAIPFPHRAG ILYELWYICS WEKQEDNEKHL NWIRNIYNF MTPYVSKNPR | 450 |
| XAYLNYRDLD IGXNXPKNPN NYTQARIWGE KYFGKNFDRL VKVKTLVDPN | 500 |
| NFFRNEQSIP PLPRHRHGHH HHHH | 524 |

As described herein, the functionality of the OXC154 mutants were tested. This allowed for the rapid and robust identification of improvements to the catalytic conversion of CBDa or other products. The mutants were then tested combinatorially in vivo in S. cerevisiae to develop a consolidated cannabinoid producing strain.

### EXAMPLES

In overview, Examples 1 to 5 are provided.

Table 1-A shows a general screening data summary for Examples 1 to 4, designating mutagenesis technique used, library genetic manipulation, the OXC template in the Example, and the background strain.

TABLE 1-A
OXC Screening Data Summary for Examples

| Exam- | Mutagenesis | Library genetic | OXC | Background |
|---|---|---|---|---|
| ple | technique | manipulation | template | strain |
| 1 | Combinatorial | Cytosolic plasmid | OXC154 | HB965 |
| 2 | Combinatorial | Cytosolic plasmid | OXC161 | HB2191 |
| 3 | SSM* | Cytosolic plasmid | OXC158 | HB2652 |
| 4 | Combinatorial | Genome integration | OXC158 | HB3192 |

*SSM = Site-saturation mutagenesis

### Example 1

Combinatorial Set of OXC154 Mutants

Wild type cannabidiolic acid synthase (CBDa synthase or “OXC52” herein), when modified with the insertion of a serine between positions 224 and 225 in the OXC52 sequence, results in a new protein, referenced herein interchangeably as “OXC154”. This modified cannabidiolic acid synthase, OXC154, leads to significantly improved CBDa production as compared with OXC52. OXC154 is described in Applicant's co-pending application PCT/CA2020/050687, which is herein incorporated by reference. Variants of OXC154 are described herein that have increased CBDa synthase activity and/or decreased tetrahydrocannabinolic acid (THCa) synthase activity. In this example OXC154 enzyme and variants thereof are prepared.

Materials and Methods:

Genetic Manipulations:

Vector VB40 was used to construct all expression plasmids encoding enzyme proteins disclosed herein, including OXC154 and variants.

The expression plasmid encoding OXC154 was constructed by an in-house site-directed mutagenesis method, such that a serine was inserted between residues P224 and K225 relative to the wild type (OXC52) sequence (SEQ ID NO:140).

The OXC154 variants were constructed in a combinatorial library using mutations that were initially selected in a site-saturation mutagenesis library screen. The VB40 plasmid harboring OXC154 coding sequence (plasmid ID PLAS513) was used as the template in all library construction.

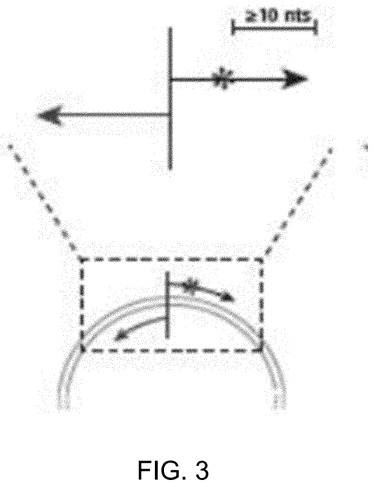

Site-saturation mutagenesis was conducted at each amino acid position by a PCR reaction using a forward degenerate NNK primer and a ‘back-to-back’ reverse non-mutagenic primer (FIG. 3). The PCR products were then processed through in vitro kinase-ligase-Dpnl reactions and transformed into Escherichia coli DH5alpha strain for amplification.

FIG. 3 illustrates PCR primers used in site-saturation mutagenesis protocol. Right-facing arrows represents forward degenerate NNK primer, symbol * denotes the mutational position, and the left-facing arrows represent a reverse primer designed ‘back-to-back’ in the opposite direction of the forward primer.

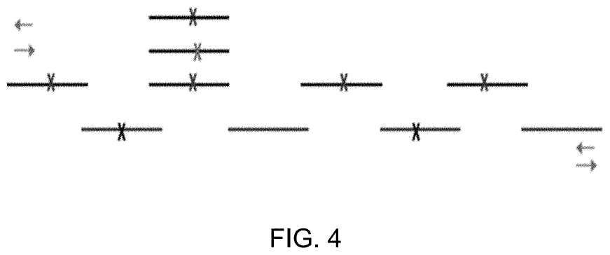

The combinatorial library was constructed by an in-house protocol. Selected mutations were combined through an overlap-extension PCR of a batch of mutagenic oligonucleotides that were generated using targeted mutagenic primers (FIG. 4). Double-stranded DNA of the assembled combinatorial mutant variants were cloned into a vector with complementary overlapping sequences, which resulted in a pool of OXC154 combinatorial variants. FIG. 4 shows an overlap-extension assembly of mutagenic oligonucleotides for combinatorial library construction. The symbol “x” represents a point mutation.

The plasmids encoding OXC154 and variant proteins as disclosed herein were transformed and expressed in Saccharomyces cerevisiae, with the host strain HB965. All DNA was transformed into background strains using the Gietz et al. transformation protocol (Gietz 2006).

Strain Growth and Media:

Strains were grown in yeast synthetic complete media with a composition of 1.7 g/L YNB without ammonium sulfate, 1.92 g/L URA dropout amino acid supplement, 1.5 g/L magnesium L-glutamate, with 2% w/v galactose, 2% w/v raffinose, 200 μg/L geneticin, and 200 μg/L ampicillin (Sigma-Aldrich Canada). The culture was incubated at 30° C. for four days (96 hours). Strain HB2010 and HB1741 were respectively used as wild type control and negative control in the screening of OXC154 variants with improved activity.

Variant Screening Conditions:

Each variant was tested in three replicates and each replicate was clonally derived from single colonies. All strains were grown in 500 μL of media for 96 hours in 96-well deepwell plates. The 96-well deepwell plates were incubated at 30° C. and shaken at 950 rpm for 96 hrs.

Metabolite extraction was performed by adding 30 μL of culture to 270 μL of 56% acetonitrile in a 96-well microtiter plate. The solutions were mixed thoroughly, then centrifuged at 3750 rpm for 10 mins. 200 μL of the soluble layer was removed and stored in a 96-well v-bottom microtiter plate. Samples were stored at −20° C. until analysis.

Quantification Protocol:

The quantification of metabolites was performed using HPLC-MS on a Acquity UPLC-TQD MS. The chromatography and MS conditions are described below.

LC Conditions

Column: ACQUITY HSS C18 UPLC 50×1 mm, 1.8 μm particle size (PN:186003529); Column temperature: 45° C.; Flow rate: 0.350 mL/min; Eluent A: Water+0.1% Formic Acid; Eluent B: ACN+0.1% Formic Acid; Gradient is shown in Table 1-B.

TABLE 1-B
Gradient

| Time (min) | % B | Flow rate (mL/min) |
|---|---|---|
| 0 | 20 | 0.350 |
| 0.60 | 98 | 0.350 |
| 1.10 | 98 | 0.350 |
| 1.11 | 20 | 0.350 |
| 1.60 | 20 | 0.350 |

ESI-MS Conditions

The following conditions were utilized: Capillary: 2.90 (kV); Source temperature: 150° C.; Desolvation gas temperature: 250° C.; Desolvation gas flow (nitrogen): 500 L/hour; Cone gas flow (nitrogen): 1 L/hour; Collision gas flow (argon): 0.10 mL/min. Detection parameters are shown in Table 2.

TABLE 2
Detection Parameters

| | OVLa | OVL | CBGa | CBDa | THCa |
|---|---|---|---|---|---|
| Retention time (min) | 0.70 | 0.72 | 0.98 | 0.98 | 1.12 |
| Parent (m/z) | 223.0 | 181.1 | 359.2 | 357.2 | 357.2 |
| Daughter (m/z) | 179.0 | 71.0 | 341.2 | 245.2 | 313.2 |
| Mode | ES−, MRM | ES+, MRM | ES−, MRM | ES−, MRM | ES−, MRM |
| Cone (V) | 35 | 20 | 40 | 45 | 45 |
| Collision (V) | 20 | 12 | 25 | 35 | 30 |

Strains used are described in Table 3.

TABLE 3
Strains Used

| Strain # | Background | Plasmids | Genotype | Notes |
|---|---|---|---|---|
| HB42 | -URA, -LEU | None | Saccharomyces cerevisiae | Base strain |
| | | | CEN.PK2; ΔLEU2; ΔURA3; Erg20K197E::KanMx | |
| HB965 | -URA, -LEU | None | Saccharomyces cerevisiae | Base strain |
| | | | CEN.PK2; ΔLEU2; ΔURA3; Erg20K197E::KanMx; ALD6; | |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| HB1741 | -URA, -LEU | PLAS416 | Saccharomyces cerevisiae | RFP |
| | | | CEN.PK2; ΔLEU2; ΔURA3; Erg20K197E::KanMx; ALD6; | mScarlet |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| HB2010 | -URA, -LEU | PLAS513 | Saccharomyces cerevisiae | OXC154 |
| | | | CEN.PK2; ΔLEU2; ΔURA3; Erg20K197E::KanMx; ALD6; | |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254; | |
| HB1973 | -URA, -LEU | PLAS462 | Saccharomyces cerevisiae | OXC154- |
| | | | CEN.PK2; ΔLEU2; ΔURA3; Erg20K197E::KanMx; ALD6; | S88A/L451G |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254; | |
| PLT1504-H11 | -URA, -LEU | PLAS-564 | Saccharomyces cerevisiae | OXC154-R3G(=GGG)/ |
| | | | CEN.PK2; ΔLEU2; ΔURA3; Erg20K197E::KanMx; ALD6; | L21G/S60T/S88A(=GCT) |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1504-C8 | -URA, -LEU | PLAS-565 | Saccharomyces cerevisiae | OXC154-R3G(=GGG)/ |
| | | | CEN.PK2; ΔLEU2; ΔURA3; Erg20K197E::KanMx; ALD6; | A18E/T49R/S60T/ |
| | | | ASC1L641P; NPGA; MAF1; PGK1p: Acc1; tHMGR1; IDI; | S88A(=GCT) |
| | | | DiPKS_G1516R X 5; ACC1_S659A_S1157A; UBI4p: | |
| | | | ERG20; pGAL: OAC; pGAL: PT254 | |
| PLT1504-F12 | -URA, -LEU | PLAS-566 | Saccharomyces cerevisiae | OXC154-R3T/ |
| | | | CEN.PK2; ΔLEU2; ΔURA3; Erg20K197E::KanMx; ALD6; | T49R/S88A(=GCC) |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1505-D11 | -URA, -LEU | PLAS-567 | Saccharomyces cerevisiae | OXC154-R3W/ |
| | | | CEN.PK2; ΔLEU2; ΔURA3; Erg20K197E::KanMx; ALD6; | A18E/T49R/S60T/ |
| | | | ASC1L641P; NPGA; MAF1; PGK1p: Acc1; tHMGR1; IDI; | S88A(=GCT) |
| | | | DiPKS_G1516R X 5; ACC1_S659A_S1157A; UBI4p: | |
| | | | ERG20; pGAL: OAC; pGAL: PT254 | |
| PLT1505-C9 | -URA, -LEU | PLAS-568 | Saccharomyces cerevisiae | OXC154-R3V/ |
| | | | CEN.PK2; ΔLEU2; ΔURA3; Erg20K197E::KanMx; ALD6; | T49R/S60T/S88A |
| | | | ASC1L641P; NPGA; MAF1; PGK1p: Acc1; tHMGR1; IDI; | (=GCT) |
| | | | DiPKS_G1516R X 5; ACC1_S659A_S1157A; UBI4p: | |
| | | | ERG20; pGAL: OAC; pGAL: PT254 | |
| PLT1505-H5 | -URA, -LEU | PLAS-569 | Saccharomyces cerevisiae | OXC154-R3V/ |
| | | | CEN.PK2; ΔLEU2; ΔURA3; Erg20K197E::KanMx; ALD6; | T49R/S60T/S88A |
| | | | ASC1L641P; NPGA; MAF1; PGK1p: Acc1; tHMGR1; IDI; | (=GCC) |
| | | | DiPKS_G1516R X 5; ACC1_S659A_S1157A; UBI4p: | |
| | | | ERG20; pGAL: OAC; pGAL: PT254 | |
| PLT1504-G7 | -URA, -LEU | PLAS-570 | Saccharomyces cerevisiae | OXC154- |
| | | | CEN.PK2; ΔLEU2; ΔURA3; Erg20K197E::KanMx; ALD6; | A18A(=GCC) |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1504-E3 | -URA, -LEU | PLAS-571 | Saccharomyces cerevisiae | OXC154-R3T/ |
| | | | CEN.PK2; ΔLEU2; ΔURA3; Erg20K197E::KanMx; ALD6; | A18E/T49R/ |
| | | | ASC1L641P; NPGA; MAF1; PGK1p: Acc1; tHMGR1; IDI; | S88A(=GCC) |
| | | | DiPKS_G1516R X 5; ACC1_S659A_S1157A; UBI4p: | |
| | | | ERG20; pGAL: OAC; pGAL: PT254 | |
| PLT1505-B8 | -URA, -LEU | PLAS-572 | Saccharomyces cerevisiae | OXC154-R3T/ |
| | | | CEN.PK2; ΔLEU2; ΔURA3; Erg20K197E::KanMx; ALD6; | S88A(=GCC) |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1505-B9 | -URA, -LEU | PLAS-573 | Saccharomyces cerevisiae | OXC154- |
| | | | CEN.PK2; ΔLEU2; ΔURA3; Erg20K197E::KanMx; ALD6; | R3G(=GGG)/ |
| | | | ASC1L641P; NPGA; MAF1; PGK1p: Acc1; tHMGR1; IDI; | L21G/T49R |
| | | | DiPKS_G1516R X 5; ACC1_S659A_S1157A; UBI4p: | |
| | | | ERG20; pGAL: OAC; pGAL: PT254 | |
| PLT1504-C11 | -URA, -LEU | PLAS-574 | Saccharomyces cerevisiae | OXC154-R3T/ |
| | | | CEN.PK2; ΔLEU2; ΔURA3; Erg20K197E::KanMx; ALD6; | T49R/ |
| | | | ASC1L641P; NPGA; MAF1; PGK1p: Acc1; tHMGR1; IDI; | S88A(=GCT) |
| | | | DiPKS_G1516R X 5; ACC1_S659A_S1157A; UBI4p: | |
| | | | ERG20; pGAL: OAC; pGAL: PT254 | |
| PLT1504-F5 | -URA, -LEU | PLAS-575 | Saccharomyces cerevisiae | OXC154- |
| | | | CEN.PK2; ΔLEU2; ΔURA3; Erg20K197E::KanMx; ALD6; | R3G(=GGA)/ |
| | | | ASC1L641P; NPGA; MAF1; PGK1p: Acc1; tHMGR1; IDI; | A18E/T49R/ |
| | | | DiPKS_G1516R X 5; ACC1_S659A_S1157A; UBI4p: | S60T/ |
| | | | ERG20; pGAL: OAC; pGAL: PT254 | S88A(=GCC) |
| PLT1506-C5 | -URA, -LEU | PLAS-576 | Saccharomyces cerevisiae | OXC154-R3W/ |
| | | | CEN.PK2; ΔLEU2; ΔURA3; Erg20K197E::KanMx; ALD6; | T49R/ |
| | | | ASC1L641P; NPGA; MAF1; PGK1p: Acc1; tHMGR1; IDI; | S88A(=GCC)/ |
| | | | DiPKS_G1516R X 5; ACC1_S659A_S1157A; UBI4p: | V97E |
| | | | ERG20; pGAL: OAC; pGAL: PT254 | |
| PLT1505-F12 | -URA, -LEU | PLAS-577 | Saccharomyces cerevisiae | OXC154- |
| | | | CEN.PK2; ΔLEU2; ΔURA3; Erg20K197E::KanMx; ALD6; | R3G(=GGG)/ |
| | | | ASC1L641P; NPGA; MAF1; PGK1p: Acc1; tHMGR1; IDI; | A18E/ |
| | | | DiPKS_G1516R X 5; ACC1_S659A_S1157A; UBI4p: | S88A(=GCC) |
| | | | ERG20; pGAL: OAC; pGAL: PT254 | |
| PLT1504-D6 | -URA, -LEU | PLAS-578 | Saccharomyces cerevisiae | OXC154- |
| | | | CEN.PK2; ΔLEU2; ΔURA3; Erg20K197E::KanMx; ALD6; | R3V/A18E/ |
| | | | ASC1L641P; NPGA; MAF1; PGK1p: Acc1; tHMGR1; IDI; | T49R/S60T/ |
| | | | DiPKS_G1516R X 5; ACC1_S659A_S1157A; UBI4p: | S88A(=GCC) |
| | | | ERG20; pGAL: OAC; pGAL: PT254 | |
| PLT1504-A2 | -URA, -LEU | PLAS-579 | Saccharomyces cerevisiae | OXC154- |
| | | | CEN.PK2; ΔLEU2; ΔURA3; Erg20K197E::KanMx; ALD6; | S60T/S88A |
| | | | ASC1L641P; NPGA; MAF1; PGK1p: Acc1; tHMGR1; IDI; | (=GCC) |
| | | | DiPKS_G1516R X 5; ACC1_S659A_S1157A; UBI4p: | |
| | | | ERG20; pGAL: OAC; pGAL: PT254 | |
| PLT1505-E11 | -URA, -LEU | PLAS-580 | Saccharomyces cerevisiae | OXC154- |
| | | | CEN.PK2; ΔLEU2; ΔURA3; Erg20K197E::KanMx; ALD6; | R3T/A18E/ |
| | | | ASC1L641P; NPGA; MAF1; PGK1p: Acc1; tHMGR1; IDI; | T49R/S60T/ |
| | | | DiPKS_G1516R X 5; ACC1_S659A_S1157A; UBI4p: | S88A(=GCT) |
| | | | ERG20; pGAL: OAC; pGAL: PT254 | |
| PLT1505-F9 | -URA, -LEU | PLAS-581 | Saccharomyces cerevisiae | OXC154- R3W/ |
| | | | CEN.PK2; ΔLEU2; ΔURA3; Erg20K197E::KanMx; ALD6; | L21G/T49R/ |
| | | | ASC1L641P; NPGA; MAF1; PGK1p: Acc1; tHMGR1; IDI; | S88A(=GCC)/ |
| | | | DiPKS_G1516R X 5; ACC1_S659A_S1157A; UBI4p: | V97E |
| | | | ERG20; pGAL: OAC; pGAL: PT254 | |
| PLT1506-A2 | -URA, -LEU | PLAS-582 | Saccharomyces cerevisiae | OXC154- |
| | | | CEN.PK2; ΔLEU2; ΔURA3; Erg20K197E::KanMx; ALD6; | R3T/A18E/ |
| | | | ASC1L641P; NPGA; MAF1; PGK1p: Acc1; tHMGR1; IDI; | T49R/S60T |
| | | | DiPKS_G1516R X 5; ACC1_S659A_S1157A; UBI4p: | |
| | | | ERG20; pGAL: OAC; pGAL: PT254 | |
| PLT1504-D12 | -URA, -LEU | PLAS-583 | Saccharomyces cerevisiae | OXC154- |
| | | | CEN.PK2; ΔLEU2; ΔURA3; Erg20K197E::KanMx; ALD6; | P2W/T26A/ |
| | | | ASC1L641P; NPGA; MAF1; PGK1p: Acc1; tHMGR1; IDI; | S60T |
| | | | DiPKS_G1516R X 5; ACC1_S659A_S1157A; UBI4p: | |
| | | | ERG20; pGAL: OAC; pGAL: PT254 | |
| PLT1505-B11 | -URA, -LEU | PLAS-584 | Saccharomyces cerevisiae | OXC154- |
| | | | CEN.PK2; ΔLEU2; ΔURA3; Erg20K197E::KanMx; ALD6; | R3G(=GGG)/ |
| | | | ASC1L641P; NPGA; MAF1; PGK1p: Acc1; tHMGR1; IDI; | L21G/S60T/ |
| | | | DiPKS_G1516R X 5; ACC1_S659A_S1157A; UBI4p: | S88A(=GCC)/ |
| | | | ERG20; pGAL: OAC; pGAL: PT254 | V97E |
| PLT1505-G11 | -URA, -LEU | PLAS-585 | Saccharomyces cerevisiae | OXC154- |
| | | | CEN.PK2; ΔLEU2; ΔURA3; Erg20K197E::KanMx; ALD6; | R3G(=GGG)/ |
| | | | ASC1L641P; NPGA; MAF1; PGK1p: Acc1; tHMGR1; IDI; | A18E/T49R/ |
| | | | DiPKS_G1516R X 5; ACC1_S659A_S1157A; UBI4p: | S88A(=GCC) |
| | | | ERG20; pGAL: OAC; pGAL: PT254 | |
| PLT1506-A10 | -URA, -LEU | PLAS-586 | Saccharomyces cerevisiae | OXC154- |
| | | | CEN.PK2; ΔLEU2; ΔURA3; Erg20K197E::KanMx; ALD6; | R3T/L21G/ |
| | | | ASC1L641P; NPGA; MAF1; PGK1p: Acc1; tHMGR1; IDI; | S60T/S88A |
| | | | DiPKS_G1516R X 5; ACC1_S659A_S1157A; UBI4p: | (=GCC)/ |
| | | | ERG20; pGAL: OAC; pGAL: PT254 | V97D |
| PLT1505-H3 | -URA, -LEU | PLAS-587 | Saccharomyces cerevisiae | OXC154- P2W/ |
| | | | CEN.PK2; ΔLEU2; ΔURA3; Erg20K197E::KanMx; ALD6; | L21G/T49R/ |
| | | | ASC1L641P; NPGA; MAF1; PGK1p: Acc1; tHMGR1; IDI; | S88A(=GCC)/ |
| | | | DiPKS_G1516R X 5; ACC1_S659A_S1157A; UBI4p: | V97E |
| | | | ERG20; pGAL: OAC; pGAL: PT254 | |
| PLT1505-H2 | -URA, -LEU | PLAS-588 | Saccharomyces cerevisiae | OXC154- |
| | | | CEN.PK2; ΔLEU2; ΔURA3; Erg20K197E::KanMx; ALD6; | R3G(=GGG)/ |
| | | | ASC1L641P; NPGA; MAF1; PGK1p: Acc1; tHMGR1; IDI; | L21G/T49R/ |
| | | | DiPKS_G1516R X 5; ACC1_S659A_S1157A; UBI4p: | S88A(=GCT) |
| | | | ERG20; pGAL: OAC; pGAL: PT254 | |
| PLT1506-C4 | -URA, -LEU | PLAS-589 | Saccharomyces cerevisiae | OXC154- |
| | | | CEN.PK2; ΔLEU2; ΔURA3; Erg20K197E::KanMx; ALD6; | S295S(=TCA) |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1506-A8 | -URA, -LEU | PLAS-590 | Saccharomyces cerevisiae | OXC154- |
| | | | CEN.PK2; ΔLEU2; ΔURA3; Erg20K197E::KanMx; ALD6; | R3V/L21G/ |
| | | | ASC1L641P; NPGA; MAF1; PGK1p: Acc1; tHMGR1; IDI; | S60T/S88A |
| | | | DiPKS_G1516R X 5; ACC1_S659A_S1157A; UBI4p: | (=GCC) |
| | | | ERG20; pGAL: OAC; pGAL: PT254 | |
| PLT1505-F2 | -URA, -LEU | PLAS-591 | Saccharomyces cerevisiae | OXC154- |
| | | | CEN.PK2; ΔLEU2; ΔURA3; Erg20K197E::KanMx; ALD6; | R3T/A18E/ |
| | | | ASC1L641P; NPGA; MAF1; PGK1p: Acc1; tHMGR1; IDI; | S88A(=GCC) |
| | | | DiPKS_G1516R X 5; ACC1_S659A_S1157A; UBI4p: | |
| | | | ERG20; pGAL: OAC; pGAL: PT254 | |
| PLT1504-E2 | -URA, -LEU | PLAS-592 | Saccharomyces cerevisiae | OXC154- |
| | | | CEN.PK2; ΔLEU2; ΔURA3; Erg20K197E::KanMx; ALD6; | S60T/S88A |
| | | | ASC1L641P; NPGA; MAF1; PGK1p: Acc1; tHMGR1; IDI; | (=GCT) |
| | | | DiPKS_G1516R X 5; ACC1_S659A_S1157A; UBI4p: | |
| | | | ERG20; pGAL: OAC; pGAL: PT254 | |
| PLT1506-H2 | -URA, -LEU | PLAS-593 | Saccharomyces cerevisiae | OXC154- |
| | | | CEN.PK2; ΔLEU2; ΔURA3; Erg20K197E::KanMx; ALD6; | R3W/T49R/ |
| | | | ASC1L641P; NPGA; MAF1; PGK1p: Acc1; tHMGR1; IDI; | S88A(=GCT) |
| | | | DiPKS_G1516R X 5; ACC1_S659A_S1157A; UBI4p: | |
| | | | ERG20; pGAL: OAC; pGAL: PT254 | |
| PLT1505-A6 | -URA, -LEU | PLAS-594 | Saccharomyces cerevisiae | OXC154-T49R/ |
| | | | CEN.PK2; ΔLEU2; ΔURA3; Erg20K197E::KanMx; ALD6; | S88A(=GCC) |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1506-F10 | -URA, -LEU | PLAS-595 | Saccharomyces cerevisiae | OXC154- |
| | | | CEN.PK2; ΔLEU2; ΔURA3; Erg20K197E::KanMx; ALD6; | R3W/S47F |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1508-H10 | -URA, -LEU | PLAS-596 | Saccharomyces cerevisiae | OXC154- |
| | | | CEN.PK2; ΔLEU2; ΔURA3; Erg20K197E::KanMx; ALD6; | A347G/I372L/ |
| | | | ASC1L641P; NPGA; MAF1; PGK1p: Acc1; tHMGR1; IDI; | L451G |
| | | | DiPKS_G1516R X 5; ACC1_S659A_S1157A; UBI4p: | |
| | | | ERG20; pGAL: OAC; pGAL: PT254 | |
| PLT1505-E9 | -URA, -LEU | PLAS-597 | Saccharomyces cerevisiae | OXC154- |
| | | | CEN.PK2; ΔLEU2; ΔURA3; Erg20K197E::KanMx; ALD6; | R3G(=GGG)/ |
| | | | ASC1L641P; NPGA; MAF1; PGK1p: Acc1; tHMGR1; IDI; | L21G/S60T |
| | | | DiPKS_G1516R X 5; ACC1_S659A_S1157A; UBI4p: | |
| | | | ERG20; pGAL: OAC; pGAL: PT254 | |
| PLT1506-C10 | -URA, -LEU | PLAS-598 | Saccharomyces cerevisiae | OXC154- |
| | | | CEN.PK2; ΔLEU2; ΔURA3; Erg20K197E::KanMx; ALD6; | R3T/L21G/ |
| | | | ASC1L641P; NPGA; MAF1; PGK1p: Acc1; tHMGR1; IDI; | T49R/ |
| | | | DiPKS_G1516R X 5; ACC1_S659A_S1157A; UBI4p: | S88A(=GCT) |
| | | | ERG20; pGAL: OAC; pGAL: PT254 | |
| PLT1506-C4 | -URA, -LEU | PLAS-599 | Saccharomyces cerevisiae | OXC154- |
| | | | CEN.PK2; ΔLEU2; ΔURA3; Erg20K197E::KanMx; ALD6; | R3T/L21G/ |
| | | | ASC1L641P; NPGA; MAF1; PGK1p: Acc1; tHMGR1; IDI; | S60T |
| | | | DiPKS_G1516R X 5; ACC1_S659A_S1157A; UBI4p: | |
| | | | ERG20; pGAL: OAC; pGAL: PT254 | |
| PLT1506-A3 | -URA, -LEU | PLAS-600 | Saccharomyces cerevisiae | OXC154-R3W/ |
| | | | CEN.PK2; ΔLEU2; ΔURA3; Erg20K197E::KanMx; ALD6; | L21G/ |
| | | | ASC1L641P; NPGA; MAF1; PGK1p: Acc1; tHMGR1; IDI; | S88A(=GCT) |
| | | | DiPKS_G1516R X 5; ACC1_S659A_S1157A; UBI4p: | |
| | | | ERG20; pGAL: OAC; pGAL: PT254 | |
| PLT1506-D11 | -URA, -LEU | PLAS-601 | Saccharomyces cerevisiae | OXC154- |
| | | | CEN.PK2; ΔLEU2; ΔURA3; Erg20K197E::KanMx; ALD6; | L21G/T49R/ |
| | | | ASC1L641P; NPGA; MAF1; PGK1p: Acc1; tHMGR1; IDI; | S60T/S88A(=GCT) |
| | | | DiPKS_G1516R X 5; ACC1_S659A_S1157A; UBI4p: | |
| | | | ERG20; pGAL: OAC; pGAL: PT254 | |
| PLT1507-B11 | -URA, -LEU | PLAS-602 | Saccharomyces cerevisiae | OXC154- |
| | | | CEN.PK2; ΔLEU2; ΔURA3; Erg20K197E::KanMx; ALD6; | A347G/A383V |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1505-A9 | -URA, -LEU | PLAS-603 | Saccharomyces cerevisiae | OXC154- |
| | | | CEN.PK2; ΔLEU2; ΔURA3; Erg20K197E::KanMx; ALD6; | R3W/L21G/ |
| | | | ASC1L641P; NPGA; MAF1; PGK1p: Acc1; tHMGR1; IDI; | T49R/S60T/ |
| | | | DiPKS_G1516R X 5; ACC1_S659A_S1157A; UBI4p: | S88A(=GCT) |
| | | | ERG20; pGAL: OAC; pGAL: PT254 | |
| PLT1505-E12 | -URA, -LEU | PLAS-604 | Saccharomyces cerevisiae | OXC154- |
| | | | CEN.PK2; ΔLEU2; ΔURA3; Erg20K197E::KanMx; ALD6; | A18E/ |
| | | | ASC1L641P; NPGA; MAF1; PGK1p: Acc1; tHMGR1; IDI; | S88A(=GCC) |
| | | | DiPKS_G1516R X 5; ACC1_S659A_S1157A; UBI4p: | |
| | | | ERG20; pGAL: OAC; pGAL: PT254 | |
| PLT1506-H9 | -URA, -LEU | PLAS-605 | Saccharomyces cerevisiae | OXC154- |
| | | | CEN.PK2; ΔLEU2; ΔURA3; Erg20K197E::KanMx; ALD6; | R3W/L21G/ |
| | | | ASC1L641P; NPGA; MAF1; PGK1p: Acc1; tHMGR1; IDI; | T49R |
| | | | DiPKS_G1516R X 5; ACC1_S659A_S1157A; UBI4p: | |
| | | | ERG20; pGAL: OAC; pGAL: PT254 | |
| PLT1508-E1 | -URA, -LEU | PLAS-606 | Saccharomyces cerevisiae | OXC154- |
| | | | CEN.PK2; ΔLEU2; ΔURA3; Erg20K197E::KanMx; ALD6; | A347G/L451G |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1507-F10 | -URA, -LEU | PLAS-607 | Saccharomyces cerevisiae | OXC154- |
| | | | CEN.PK2; ΔLEU2; ΔURA3; Erg20K197E::KanMx; ALD6; | A347G/I372L/ |
| | | | ASC1L641P; NPGA; MAF1; PGK1p: Acc1; tHMGR1; IDI; | A383V/ |
| | | | DiPKS_G1516R X 5; ACC1_S659A_S1157A; UBI4p: | L451G |
| | | | ERG20; pGAL: OAC; pGAL: PT254 | |
| PLT1508-F7 | -URA, -LEU | PLAS-608 | Saccharomyces cerevisiae | OXC154- |
| | | | CEN.PK2; ΔLEU2; ΔURA3; Erg20K197E::KanMx; ALD6; | I372L/A383V/ |
| | | | ASC1L641P; NPGA; MAF1; PGK1p: Acc1; tHMGR1; IDI; | L451G |
| | | | DiPKS_G1516R X 5; ACC1_S659A_S1157A; UBI4p: | |
| | | | ERG20; pGAL: OAC; pGAL: PT254 | |
| PLT1506-A11 | -URA, -LEU | PLAS-609 | Saccharomyces cerevisiae | OXC154- |
| | | | CEN.PK2; ΔLEU2; ΔURA3; Erg20K197E::KanMx; ALD6; | R3V/T49R/ |
| | | | ASC1L641P; NPGA; MAF1; PGK1p: Acc1; tHMGR1; IDI; | S88A(=GCT) |
| | | | DiPKS_G1516R X 5; ACC1_S659A_S1157A; UBI4p: | |
| | | | ERG20; pGAL: OAC; pGAL: PT254 | |
| PLT1504-E5 | -URA, -LEU | PLAS-610 | Saccharomyces cerevisiae | OXC154- |
| | | | CEN.PK2; ΔLEU2; ΔURA3; Erg20K197E::KanMx; ALD6; | R3G(=GGG)/ |
| | | | ASC1L641P; NPGA; MAF1; PGK1p: Acc1; tHMGR1; IDI; | A18E/S60T |
| | | | DiPKS_G1516R X 5; ACC1_S659A_S1157A; UBI4p: | |
| | | | ERG20; pGAL: OAC; pGAL: PT254 | |
| PLT1508-D10 | -URA, -LEU | PLAS-611 | Saccharomyces cerevisiae | OXC154- |
| | | | CEN.PK2; ΔLEU2; ΔURA3; Erg20K197E::KanMx; ALD6; | A347G/I372L/ |
| | | | ASC1L641P; NPGA; MAF1; PGK1p: Acc1; tHMGR1; IDI; | A383V |
| | | | DiPKS_G1516R X 5; ACC1_S659A_S1157A; UBI4p: | |
| | | | ERG20; pGAL: OAC; pGAL: PT254 | |
| PLT1505-B2 | -URA, -LEU | PLAS-612 | Saccharomyces cerevisiae | OXC154- |
| | | | CEN.PK2; ΔLEU2; ΔURA3; Erg20K197E::KanMx; ALD6; | R3T |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1505-G1 | -URA, -LEU | PLAS-613 | Saccharomyces cerevisiae | OXC154- |
| | | | CEN.PK2; ΔLEU2; ΔURA3; Erg20K197E::KanMx; ALD6; | R3V/A18E/ |
| | | | ASC1L641P; NPGA; MAF1; PGK1p: Acc1; tHMGR1; IDI; | T49R/V97E |
| | | | DiPKS_G1516R X 5; ACC1_S659A_S1157A; UBI4p: | |
| | | | ERG20; pGAL: OAC; pGAL: PT254 | |
| PLT1505-G3 | -URA, -LEU | PLAS-614 | Saccharomyces cerevisiae | OXC154- |
| | | | CEN.PK2; ΔLEU2; ΔURA3; Erg20K197E::KanMx; ALD6; | R3T/L21G/ |
| | | | ASC1L641P; NPGA; MAF1; PGK1p: Acc1; tHMGR1; IDI; | T49R/S60T/ |
| | | | DiPKS_G1516R X 5; ACC1_S659A_S1157A; UBI4p: | S88A(=GCT) |
| | | | ERG20; pGAL: OAC; pGAL: PT254 | |
| PLT1504-F6 | -URA, -LEU | PLAS-615 | Saccharomyces cerevisiae | OXC154- |
| | | | CEN.PK2; ΔLEU2; ΔURA3; Erg20K197E::KanMx; ALD6; | R3T/L21G/ |
| | | | ASC1L641P; NPGA; MAF1; PGK1p: Acc1; tHMGR1; IDI; | T49R/V97E |
| | | | DiPKS_G1516R X 5; ACC1_S659A_S1157A; UBI4p: | |
| | | | ERG20; pGAL: OAC; pGAL: PT254 | |
| PLT1505-E3 | -URA, -LEU | PLAS-616 | Saccharomyces cerevisiae | OXC154- |
| | | | CEN.PK2; ΔLEU2; ΔURA3; Erg20K197E::KanMx; ALD6; | R3V/L21G/ |
| | | | ASC1L641P; NPGA; MAF1; PGK1p: Acc1; tHMGR1; IDI; | T49R/S60T |
| | | | DiPKS_G1516R X 5; ACC1_S659A_S1157A; UBI4p: | |
| | | | ERG20; pGAL: OAC; pGAL: PT254 | |
| PLT1509-A6 | -URA, -LEU | PLAS-617 | Saccharomyces cerevisiae | OXC154- |
| | | | CEN.PK2; ΔLEU2; ΔURA3; Erg20K197E::KanMx; ALD6; | G351I/I372L |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1508-E5 | -URA, -LEU | PLAS-618 | Saccharomyces cerevisiae | OXC154- |
| | | | CEN.PK2; ΔLEU2; ΔURA3; Erg20K197E::KanMx; ALD6; | G351I/A383V/ |
| | | | ASC1L641P; NPGA; MAF1; PGK1p: Acc1; tHMGR1; IDI; | L451G |
| | | | DiPKS_G1516R X 5; ACC1_S659A_S1157A; UBI4p: | |
| | | | ERG20; pGAL: OAC; pGAL: PT254 | |
| PLT1508-F5 | -URA, -LEU | PLAS-619 | Saccharomyces cerevisiae | OXC154- |
| | | | CEN.PK2; ΔLEU2; ΔURA3; Erg20K197E::KanMx; ALD6; | G351R/I372L/ |
| | | | ASC1L641P; NPGA; MAF1; PGK1p: Acc1; tHMGR1; IDI; | L451G |
| | | | DiPKS_G1516R X 5; ACC1_S659A_S1157A; UBI4p: | |
| | | | ERG20; pGAL: OAC; pGAL: PT254 | |
| PLT1509-A10 | -URA, -LEU | PLAS-620 | Saccharomyces cerevisiae | OXC154- |
| | | | CEN.PK2; ΔLEU2; ΔURA3; Erg20K197E::KanMx; ALD6; | G351I/I372L/ |
| | | | ASC1L641P; NPGA; MAF1; PGK1p: Acc1; tHMGR1; IDI; | A383V/L451G |
| | | | DiPKS_G1516R X 5; ACC1_S659A_S1157A; UBI4p: | |
| | | | ERG20; pGAL: OAC; pGAL: PT254 | |
| PLT1508-A10 | -URA, -LEU | PLAS-621 | Saccharomyces cerevisiae | OXC154- |
| | | | CEN.PK2; ΔLEU2; ΔURA3; Erg20K197E::KanMx; ALD6; | G351R/I372L/ |
| | | | ASC1L641P; NPGA; MAF1; PGK1p: Acc1; tHMGR1; IDI; | A383V/L451G |
| | | | DiPKS_G1516R X 5; ACC1_S659A_S1157A; UBI4p: | |
| | | | ERG20; pGAL: OAC; pGAL: PT254 | |
| PLT1507-E8 | -URA, -LEU | PLAS-622 | Saccharomyces cerevisiae | OXC154- |
| | | | CEN.PK2; ΔLEU2; ΔURA3; Erg20K197E::KanMx; ALD6; | G351I/I372L/ |
| | | | ASC1L641P; NPGA; MAF1; PGK1p: Acc1; tHMGR1; IDI; | A383V |
| | | | DiPKS_G1516R X 5; ACC1_S659A_S1157A; UBI4p: | |
| | | | ERG20; pGAL: OAC; pGAL: PT254 | |
| PLT1507-B8 | -URA, -LEU | PLAS-623 | Saccharomyces cerevisiae | OXC154- |
| | | | CEN.PK2; ΔLEU2; ΔURA3; Erg20K197E::KanMx; ALD6; | N331G/ |
| | | | ASC1L641P; NPGA; MAF1; PGK1p: Acc1; tHMGR1; IDI; | Q349G/I372L/ |
| | | | DiPKS_G1516R X 5; ACC1_S659A_S1157A; UBI4p: | L451G |
| | | | ERG20; pGAL: OAC; pGAL: PT254 | |
| PLT1508-A6 | -URA, -LEU | PLAS-624 | Saccharomyces cerevisiae | OXC154- |
| | | | CEN.PK2; ΔLEU2; ΔURA3; Erg20K197E::KanMx; ALD6; | G351R/ |
| | | | ASC1L641P; NPGA; MAF1; PGK1p: Acc1; tHMGR1; IDI; | A383V/L451G |
| | | | DiPKS_G1516R X 5; ACC1_S659A_S1157A; UBI4p: | |
| | | | ERG20; pGAL: OAC; pGAL: PT254 | |
| PLT1508-H4 | -URA, -LEU | PLAS-625 | Saccharomyces cerevisiae | OXC154- |
| | | | CEN.PK2; ΔLEU2; ΔURA3; Erg20K197E::KanMx; ALD6; | Q349G/ |
| | | | ASC1L641P; NPGA; MAF1; PGK1p: Acc1; tHMGR1; IDI; | A383V/L451G |
| | | | DiPKS_G1516R X 5; ACC1_S659A_S1157A; UBI4p: | |
| | | | ERG20; pGAL: OAC; pGAL: PT254 | |
| PLT1507-E9 | -URA, -LEU | PLAS-626 | Saccharomyces cerevisiae | OXC154- |
| | | | CEN.PK2; ΔLEU2; ΔURA3; Erg20K197E::KanMx; ALD6; | A383V/ |
| | | | ASC1L641P; NPGA; MAF1; PGK1p: Acc1; tHMGR1; IDI; | L451G |
| | | | DiPKS_G1516R X 5; ACC1_S659A_S1157A; UBI4p: | |
| | | | ERG20; pGAL: OAC; pGAL: PT254 | |
| PLT1507-E12 | -URA, -LEU | PLAS-627 | Saccharomyces cerevisiae | OXC154- |
| | | | CEN.PK2; ΔLEU2; ΔURA3; Erg20K197E::KanMx; ALD6; | N331G/ |
| | | | ASC1L641P; NPGA; MAF1; PGK1p: Acc1; tHMGR1; IDI; | Q349G |
| | | | DiPKS_G1516R X 5; ACC1_S659A_S1157A; UBI4p: | |
| | | | ERG20; pGAL: OAC; pGAL: PT254 | |
| PLT1507-B3 | -URA, -LEU | PLAS-628 | Saccharomyces cerevisiae | OXC154- |
| | | | CEN.PK2; ΔLEU2; ΔURA3; Erg20K197E::KanMx; ALD6; | G351I |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1509-A11 | -URA, -LEU | PLAS-629 | Saccharomyces cerevisiae | OXC154- |
| | | | CEN.PK2; ΔLEU2; ΔURA3; Erg20K197E::KanMx; ALD6; | L451G |
| | | | ASC1L641P; NPGA; MAF1; PGK1p: Acc1; tHMGR1; IDI; | |
| | | | DiPKS_G1516R X 5; ACC1_S659A_S1157A; UBI4p: | |
| | | | ERG20; pGAL: OAC; pGAL: PT254 | |
| PLT1507-H7 | -URA, -LEU | PLAS-630 | Saccharomyces cerevisiae | OXC154- |
| | | | CEN.PK2; ΔLEU2; ΔURA3; Erg20K197E::KanMx; ALD6; | N331G/G351I/ |
| | | | ASC1L641P; NPGA; MAF1; PGK1 p: Acc1; tHMGR1; IDI; | I372L/A383V |
| | | | DiPKS_G1516R X 5; ACC1_S659A_S1157A; UBI4p: | |
| | | | ERG20; pGAL: OAC; pGAL: PT254 | |

The following plasmids were used, as described in Table 4.

TABLE 4
Plasmids

| | Plasmid | | | |
|---|---|---|---|---|
| # | Name | SEQ ID NO: | Description | Selection |
| 1 | PLAS415 | SEQ ID NO: 1 | Ostl-pro-alpha-f(I)-OXC52-VB40 | Uracil |
| 2 | PLAS513 | SEQ ID NO: 2 | Ostl-pro-alpha-f(I)-OXC154-VB40 | Uracil |
| 3 | PLAS416 | SEQ ID NO: 3 | RFP mScarlet | RFP |
| 4 | PLAS462 | SEQ ID NO: 4 | Ostl-pro-alpha-f(I)-OXC154-S88A/L451G-VB40 | Uracil |
| 5 | PLAS-564 | SEQ ID NO: 5 | OXC154-R3G(=GGG)/L21G/S60T/S88A(=GCT)- | Uracil |
| | | | VB40 | |
| 6 | PLAS-565 | SEQ ID NO: 6 | OXC154- | Uracil |
| | | | R3G(=GGG)/A18E/T49R/S60T/S88A(=GCT)-VB40 | |
| 7 | PLAS-566 | SEQ ID NO: 7 | OXC154-R3T/T49R/S88A(=GCC)-VB40 | Uracil |
| 8 | PLAS-567 | SEQ ID NO: 8 | OXC154-R3W/A18E/T49R/S60T/S88A(=GCT)-VB40 | Uracil |
| 9 | PLAS-568 | SEQ ID NO: 9 | OXC154-R3V/T49R/S60T/S88A(=GCT)-VB40 | Uracil |
| 10 | PLAS-569 | SEQ ID NO: 10 | OXC154-R3V/T49R/S60T/S88A(=GCC)-VB40 | Uracil |
| 11 | PLAS-570 | SEQ ID NO: 11 | OXC154-A18A(=GCC)-VB40 | Uracil |
| 12 | PLAS-571 | SEQ ID NO: 12 | OXC154-R3T/A18E/T49R/S88A(=GCC)-VB40 | Uracil |
| 13 | PLAS-572 | SEQ ID NO: 13 | OXC154-R3T/S88A(=GCC)-VB40 | Uracil |
| 14 | PLAS-573 | SEQ ID NO: 14 | OXC154-R3G(=GGG)/L21G/T49R-VB40 | Uracil |
| 15 | PLAS-574 | SEQ ID NO: 15 | OXC154-R3T/T49R/S88A(=GCT)-VB40 | Uracil |
| 16 | PLAS-575 | SEQ ID NO: 16 | OXC154- | Uracil |
| | | | R3G(=GGA)/A18E/T49R/S60T/S88A(=GCC)-VB40 | |
| 17 | PLAS-576 | SEQ ID NO: 17 | OXC154-R3W/T49R/S88A(=GCC)/V97E-VB40 | Uracil |
| 18 | PLAS-577 | SEQ ID NO: 18 | OXC154-R3G(=GGG)/A18E/S88A(=GCC)-VB40 | Uracil |
| 19 | PLAS-578 | SEQ ID NO: 19 | OXC154-R3V/A18E/T49R/S60T/S88A(=GCC)-VB40 | Uracil |
| 20 | PLAS-579 | SEQ ID NO: 20 | OXC154-S60T/S88A(=GCC)-VB40 | Uracil |
| 21 | PLAS-580 | SEQ ID NO: 21 | OXC154-R3T/A18E/T49R/S60T/S88A(=GCT)-VB40 | Uracil |
| 22 | PLAS-581 | SEQ ID NO: 22 | OXC154-R3W/L21G/T49R/S88A(=GCC)/V97E- | Uracil |
| | | | VB40 | |
| 23 | PLAS-582 | SEQ ID NO: 23 | OXC154-R3T/A18E/T49R/S60T-VB40 | Uracil |
| 24 | PLAS-583 | SEQ ID NO: 24 | OXC154-P2W/T26A/S60T-VB40 | Uracil |
| 25 | PLAS-584 | SEQ ID NO: 25 | OXC154- | Uracil |
| | | | R3G(=GGG)/L21G/S60T/S88A(=GCC)/V97E-VB40 | |
| 26 | PLAS-585 | SEQ ID NO: 26 | OXC154-R3G(=GGG)/A18E/T49R/S88A(=GCC)- | Uracil |
| | | | VB40 | |
| 27 | PLAS-586 | SEQ ID NO: 27 | OXC154-R3T/L21G/S60T/S88A(=GCC)/V97D-VB40 | Uracil |
| 28 | PLAS-587 | SEQ ID NO: 28 | OXC154-P2W/L21G/T49R/S88A(=GCC)/V97E- | Uracil |
| | | | VB40 | |
| 29 | PLAS-588 | SEQ ID NO: 29 | OXC154-R3G(=GGG)/L21G/T49R/S88A(=GCT)- | Uracil |
| | | | VB40 | |
| 30 | PLAS-589 | SEQ ID NO: 30 | OXC154-S295S(=TCA)-VB40 | Uracil |
| 31 | PLAS-590 | SEQ ID NO: 31 | OXC154-R3V/L21G/S60T/S88A(=GCC)-VB40 | Uracil |
| 32 | PLAS-591 | SEQ ID NO: 32 | OXC154-R3T/A18E/S88A(=GCC)-VB40 | Uracil |
| 33 | PLAS-592 | SEQ ID NO: 33 | OXC154-S60T/S88A(=GCT)-VB40 | Uracil |
| 34 | PLAS-593 | SEQ ID NO: 34 | OXC154-R3W/T49R/S88A(=GCT)-VB40 | Uracil |
| 35 | PLAS-594 | SEQ ID NO: 35 | OXC154-T49R/S88A(=GCC)-VB40 | Uracil |
| 36 | PLAS-595 | SEQ ID NO: 36 | OXC154-R3W/S47F-VB40 | Uracil |
| 37 | PLAS-596 | SEQ ID NO: 37 | OXC154-A347G/I372L/L451G-VB40 | Uracil |
| 38 | PLAS-597 | SEQ ID NO: 38 | OXC154-R3G(=GGG)/L21G/S60T-VB40 | Uracil |
| 39 | PLAS-598 | SEQ ID NO: 39 | OXC154-R3T/L21G/T49R/S88A(=GCT)-VB40 | Uracil |
| 40 | PLAS-599 | SEQ ID NO: 40 | OXC154-R3T/L21G/S60T-VB40 | Uracil |
| 41 | PLAS-600 | SEQ ID NO: 41 | OXC154-R3W/L21G/S88A(=GCT)-VB40 | Uracil |
| 42 | PLAS-601 | SEQ ID NO: 42 | OXC154-L21G/T49R/S60T/S88A(=GCT)-VB40 | Uracil |
| 43 | PLAS-602 | SEQ ID NO: 43 | OXC154-A347G/A383V-VB40 | Uracil |
| 44 | PLAS-603 | SEQ ID NO: 44 | OXC154-R3W/L21G/T49R/S60T/S88A(=GCT)-VB40 | Uracil |
| 45 | PLAS-604 | SEQ ID NO: 45 | OXC154-A18E/S88A(=GCC)-VB40 | Uracil |
| 46 | PLAS-605 | SEQ ID NO: 46 | OXC154-R3W/L21G/T49R-VB40 | Uracil |
| 47 | PLAS-606 | SEQ ID NO: 47 | OXC154-A347G/L451G-VB40 | Uracil |
| 48 | PLAS-607 | SEQ ID NO: 48 | OXC154-A347G/I372L/A383V/L451G-VB40 | Uracil |
| 49 | PLAS-608 | SEQ ID NO: 49 | OXC154-I372L/A383V/L451G-VB40 | Uracil |
| 50 | PLAS-609 | SEQ ID NO: 50 | OXC154-R3V/T49R/S88A(=GCT)-VB40 | Uracil |
| 51 | PLAS-610 | SEQ ID NO: 51 | OXC154-R3G(=GGG)/A18E/S60T-VB40 | Uracil |
| 52 | PLAS-611 | SEQ ID NO: 52 | OXC154-A347G/I372L/A383V-VB40 | Uracil |
| 53 | PLAS-612 | SEQ ID NO: 53 | OXC154-R3T-VB40 | Uracil |
| 54 | PLAS-613 | SEQ ID NO: 54 | OXC154-R3V/A18E/T49R/V97E-VB40 | Uracil |
| 55 | PLAS-614 | SEQ ID NO: 55 | OXC154-R3T/L21G/T49R/S60T/S88A(=GCT)-VB40 | Uracil |
| 56 | PLAS-615 | SEQ ID NO: 56 | OXC154-R3T/L21G/T49R/V97E-VB40 | Uracil |
| 57 | PLAS-616 | SEQ ID NO: 57 | OXC154-R3V/L21G/T49R/S60T-VB40 | Uracil |
| 58 | PLAS-617 | SEQ ID NO: 58 | OXC154-G351I/I372L-VB40 | Uracil |
| 59 | PLAS-618 | SEQ ID NO: 59 | OXC154-G351I/A383V/L451G-VB40 | Uracil |
| 60 | PLAS-619 | SEQ ID NO: 60 | OXC154-G351R/I372L/L451G-VB40 | Uracil |
| 61 | PLAS-620 | SEQ ID NO: 61 | OXC154-G351I/I372L/A383V/L451G-VB40 | Uracil |
| 62 | PLAS-621 | SEQ ID NO: 62 | OXC154-G351R/I372L/A383V/L451G-VB40 | Uracil |
| 63 | PLAS-622 | SEQ ID NO: 63 | OXC154-G351I/I372L/A383V-VB40 | Uracil |
| 64 | PLAS-623 | SEQ ID NO: 64 | OXC154-N331G/Q349G/I372L/L451G-VB40 | Uracil |
| 65 | PLAS-624 | SEQ ID NO: 65 | OXC154-G351R/A383V/L451G-VB40 | Uracil |
| 66 | PLAS-625 | SEQ ID NO: 66 | OXC154-Q349G/A383V/L451G-VB40 | Uracil |
| 67 | PLAS-626 | SEQ ID NO: 67 | OXC154-A383V/L451G-VB40 | Uracil |
| 68 | PLAS-627 | SEQ ID NO: 68 | OXC154-N331G/Q349G-VB40 | Uracil |
| 69 | PLAS-628 | SEQ ID NO: 69 | OXC154-G351I-VB40 | Uracil |
| 70 | PLAS-629 | SEQ ID NO: 70 | OXC154-L451G-VB40 | Uracil |
| 71 | PLAS-630 | SEQ ID NO: 71 | OXC154-N331G/G351I/I372L/A383V-VB40 | Uracil |

The following sequences are described herein (Table 5). shows further sequences described herein. Assigned descriptive names for sequences indicate the starting sequence from which mutations are made, such as from “OXC154”. Where OXC154 is indicated, the listed mutated residues in the descriptive name are changed from SEQ ID NO:141, which is the mutated protein from wild type OXC52 (SEQ ID NO:140) by having a serine insertion between residues P224 and K225. For example, SEQ ID NO: 138 (Protein), is assigned “OXC154-L451G” as its descriptive name. Thus, for this sequence the mutation from SEQ ID NO:141 (OXC154) is L451G.

TABLE 5
Sequences

| | | DNA/ | Length of | Position of coding |
|---|---|---|---|---|
| SEQ ID NO: | Description | Protein | sequence | sequence |
| SEQ ID NO: 1 | Ostl-pro-alpha-f(I)-OXC52 | DNA | 7263 | 2925 to 4496 |
| SEQ ID NO: 2 | Ostl-pro-alpha-f(I)-OXC154 | DNA | 7266 | 2925 to 4499 |
| SEQ ID NO: 3 | RFP mScarlet | DNA | 6114 | 2649-3347 |
| SEQ ID NO: 4 | OXC154-S88A/L451G | DNA | 7266 | 2925 to 4499 |
| SEQ ID NO: 5 | OXC154- | DNA | 7266 | 2925 to 4499 |
| | R3G(=GGG)/L21G/S60T/S88A(=GCT) | | | |
| SEQ ID NO: 6 | OXC154- | DNA | 7266 | 2925 to 4499 |
| | R3G(=GGG)/A18E/T49R/S60T/S88A(=GCT) | | | |
| SEQ ID NO: 7 | OXC154-R3T/T49R/S88A(=GCC) | DNA | 7266 | 2925 to 4499 |
| SEQ ID NO: 8 | OXC154- | DNA | 7266 | 2925 to 4499 |
| | R3W/A18E/T49R/S60T/S88A(=GCT) | | | |
| SEQ ID NO: 9 | OXC154- | DNA | 7266 | 2925 to 4499 |
| | R3V/T49R/S60T/S88A(=GCT) | | | |
| SEQ ID NO: 10 | OXC154- | DNA | 7266 | 2925 to 4499 |
| | R3V/T49R/S60T/S88A(=GCC) | | | |
| SEQ ID NO: 11 | OXC154-A18A(=GCC) | DNA | 7266 | 2925 to 4499 |
| SEQ ID NO: 12 | OXC154- | DNA | 7266 | 2925 to 4499 |
| | R3T/A18E/T49R/S88A(=GCC) | | | |
| SEQ ID NO: 13 | OXC154-R3T/S88A(=GCC) | DNA | 7266 | 2925 to 4499 |
| SEQ ID NO: 14 | OXC154-R3G(=GGG)/L21G/T49R | DNA | 7266 | 2925 to 4499 |
| SEQ ID NO: 15 | OXC154-R3T/T49R/S88A(=GCT) | DNA | 7266 | 2925 to 4499 |
| SEQ ID NO: 16 | OXC154- | DNA | 7266 | 2925 to 4499 |
| | R3G(=GGA)/A18E/T49R/S60T/S88A(=GCC) | | | |
| SEQ ID NO: 17 | OXC154- | DNA | 7266 | 2925 to 4499 |
| | R3W/T49R/S88A(=GCC)/V97E | | | |
| SEQ ID NO: 18 | OXC154- | DNA | 7266 | 2925 to 4499 |
| | R3G(=GGG)/A18E/S88A(=GCC) | | | |
| SEQ ID NO: 19 | OXC154- | DNA | 7266 | 2925 to 4499 |
| | R3V/A18E/T49R/S60T/S88A(=GCC) | | | |
| SEQ ID NO: 20 | OXC154-S60T/S88A(=GCC) | DNA | 7266 | 2925 to 4499 |
| SEQ ID NO: 21 | OXC154- | DNA | 7266 | 2925 to 4499 |
| | R3T/A18E/T49R/S60T/S88A(=GCT) | | | |
| SEQ ID NO: 22 | OXC154- | DNA | 7266 | 2925 to 4499 |
| | R3W/L21G/T49R/S88A(=GCC)/V97E | | | |
| SEQ ID NO: 23 | OXC154-R3T/A18E/T49R/S60T | DNA | 7266 | 2925 to 4499 |
| SEQ ID NO: 24 | OXC154-P2W/T26A/S60T | DNA | 7266 | 2925 to 4499 |
| SEQ ID NO: 25 | OXC154- | DNA | 7266 | 2925 to 4499 |
| | R3G(=GGG)/L21G/S60T/S88A(=GCC)/V97E | | | |
| SEQ ID NO: 26 | OXC154- | DNA | 7266 | 2925 to 4499 |
| | R3G(=GGG)/A18E/T49R/S88A(=GCC) | | | |
| SEQ ID NO: 27 | OXC154- | DNA | 7266 | 2925 to 4499 |
| | R3T/L21G/S60T/S88A(=GCC)/V97D | | | |
| SEQ ID NO: 28 | OXC154- | DNA | 7266 | 2925 to 4499 |
| | P2W/L21G/T49R/S88A(=GCC)/V97E | | | |
| SEQ ID NO: 29 | OXC154- | DNA | 7266 | 2925 to 4499 |
| | R3G(=GGG)/L21G/T49R/S88A(=GCT) | | | |
| SEQ ID NO: 30 | OXC154-S295S(=TCA) | DNA | 7266 | 2925 to 4499 |
| SEQ ID NO: 31 | OXC154- | DNA | 7266 | 2925 to 4499 |
| | R3V/L21G/S60T/S88A(=GCC) | | | |
| SEQ ID NO: 32 | OXC154-R3T/A18E/S88A(=GCC) | DNA | 7266 | 2925 to 4499 |
| SEQ ID NO: 33 | OXC154-S60T/S88A(=GCT) | DNA | 7266 | 2925 to 4499 |
| SEQ ID NO: 34 | OXC154-R3W/T49R/S88A(=GCT) | DNA | 7266 | 2925 to 4499 |
| SEQ ID NO: 35 | OXC154-T49R/S88A(=GCC) | DNA | 7266 | 2925 to 4499 |
| SEQ ID NO: 36 | OXC154-R3W/S47F | DNA | 7266 | 2925 to 4499 |
| SEQ ID NO: 37 | OXC154-A347G/I372L/L451G | DNA | 7266 | 2925 to 4499 |
| SEQ ID NO: 38 | OXC154-R3G(=GGG)/L21G/S60T | DNA | 7266 | 2925 to 4499 |
| SEQ ID NO: 39 | OXC154- | DNA | 7266 | 2925 to 4499 |
| | R3T/L21G/T49R/S88A(=GCT) | | | |
| SEQ ID NO: 40 | OXC154-R3T/L21G/S60T | DNA | 7266 | 2925 to 4499 |
| SEQ ID NO: 41 | OXC154-R3W/L21G/S88A(=GCT) | DNA | 7266 | 2925 to 4499 |
| SEQ ID NO: 42 | OXC154- | DNA | 7266 | 2925 to 4499 |
| | L21G/T49R/S60T/S88A(=GCT) | | | |
| SEQ ID NO: 43 | OXC154-A347G/A383V | DNA | 7266 | 2925 to 4499 |
| SEQ ID NO: 44 | OXC154- | DNA | 7266 | 2925 to 4499 |
| | R3W/L21G/T49R/S60T/S88A(=GCT) | | | |
| SEQ ID NO: 45 | OXC154-A18E/S88A(=GCC) | DNA | 7266 | 2925 to 4499 |
| SEQ ID NO: 46 | OXC154-R3W/L21G/T49R | DNA | 7266 | 2925 to 4499 |
| SEQ ID NO: 47 | OXC154-A347G/L451G | DNA | 7266 | 2925 to 4499 |
| SEQ ID NO: 48 | OXC154-A347G/I372L/A383V/L451G | DNA | 7266 | 2925 to 4499 |
| SEQ ID NO: 49 | OXC154-I372L/A383V/L451G | DNA | 7266 | 2925 to 4499 |
| SEQ ID NO: 50 | OXC154-R3V/T49R/S88A(=GCT) | DNA | 7266 | 2925 to 4499 |
| SEQ ID NO: 51 | OXC154-R3G(=GGG)/A18E/S60T | DNA | 7266 | 2925 to 4499 |
| SEQ ID NO: 52 | OXC154-A347G/I372L/A383V | DNA | 7266 | 2925 to 4499 |
| SEQ ID NO: 53 | OXC154-R3T | DNA | 7266 | 2925 to 4499 |
| SEQ ID NO: 54 | OXC154-R3V/A18E/T49R/V97E | DNA | 7266 | 2925 to 4499 |
| SEQ ID NO: 55 | OXC154- | DNA | 7266 | 2925 to 4499 |
| | R3T/L21G/T49R/S60T/S88A(=GCT) | | | |
| SEQ ID NO: 56 | OXC154-R3T/L21G/T49R/V97E | DNA | 7266 | 2925 to 4499 |
| SEQ ID NO: 57 | OXC154-R3V/L21G/T49R/S60T | DNA | 7266 | 2925 to 4499 |
| SEQ ID NO: 58 | OXC154-G351I/I372L | DNA | 7266 | 2925 to 4499 |
| SEQ ID NO: 59 | OXC154-G351I/A383V/L451G | DNA | 7266 | 2925 to 4499 |
| SEQ ID NO: 60 | OXC154-G351R/I372L/L451G | DNA | 7266 | 2925 to 4499 |
| SEQ ID NO: 61 | OXC154-G351I/I372L/A383V/L451G | DNA | 7266 | 2925 to 4499 |
| SEQ ID NO: 62 | OXC154-G351R/I372L/A383V/L451G | DNA | 7266 | 2925 to 4499 |
| SEQ ID NO: 63 | OXC154-G351I/I372L/A383V | DNA | 7266 | 2925 to 4499 |
| SEQ ID NO: 64 | OXC154-N331G/Q349G/I372L/L451G | DNA | 7266 | 2925 to 4499 |
| SEQ ID NO: 65 | OXC154-G351R/A383V/L451G | DNA | 7266 | 2925 to 4499 |
| SEQ ID NO: 66 | OXC154-Q349G/A383V/L451G | DNA | 7266 | 2925 to 4499 |
| SEQ ID NO: 67 | OXC154-A383V/L451G | DNA | 7266 | 2925 to 4499 |
| SEQ ID NO: 68 | OXC154-N331G/Q349G | DNA | 7266 | 2925 to 4499 |
| SEQ ID NO: 69 | OXC154-G351I | DNA | 7266 | 2925 to 4499 |
| SEQ ID NO: 70 | OXC154-L451G | DNA | 7266 | 2925 to 4499 |
| SEQ ID NO: 71 | OXC154-N331G/G351I/I372L/A383V | DNA | 7266 | 2925 to 4499 |
| SEQ ID NO: 72 | OXC154-S88A/L451G | Protein | 524 | All |
| SEQ ID NO: 73 | OXC154- | Protein | 524 | All |
| | R3G(=GGG)/L21G/S60T/S88A(=GCT) | | | |
| SEQ ID NO: 74 | OXC154- | Protein | 524 | All |
| | R3G(=GGG)/A18E/T49R/S60T/S88A(=GCT) | | | |
| SEQ ID NO: 75 | OXC154-R3T/T49R/S88A(=GCC) | Protein | 524 | All |
| SEQ ID NO: 76 | OXC154- | Protein | 524 | All |
| | R3W/A18E/T49R/S60T/S88A(=GCT) | | | |
| SEQ ID NO: 77 | OXC154- | Protein | 524 | All |
| | R3V/T49R/S60T/S88A(=GCT) | | | |
| SEQ ID NO: 78 | OXC154- | Protein | 524 | All |
| | R3V/T49R/S60T/S88A(=GCC) | | | |
| SEQ ID NO: 79 | OXC154-A18A(=GCC) | Protein | 524 | All |
| SEQ ID NO: 80 | OXC154- | Protein | 524 | All |
| | R3T/A18E/T49R/S88A(=GCC) | | | |
| SEQ ID NO: 81 | OXC154-R3T/S88A(=GCC) | Protein | 524 | All |
| SEQ ID NO: 82 | OXC154-R3G(=GGG)/L21G/T49R | Protein | 524 | All |
| SEQ ID NO: 83 | OXC154-R3T/T49R/S88A(=GCT) | Protein | 524 | All |
| SEQ ID NO: 84 | OXC154- | Protein | 524 | All |
| | R3G(=GGA)/A18E/T49R/S60T/S88A(=GCC) | | | |
| SEQ ID NO: 85 | OXC154- | Protein | 524 | All |
| | R3W/T49R/S88A(=GCC)/V97E | | | |
| SEQ ID NO: 86 | OXC154- | Protein | 524 | All |
| | R3G(=GGG)/A18E/S88A(=GCC) | | | |
| SEQ ID NO: 87 | OXC154- | Protein | 524 | All |
| | R3V/A18E/T49R/S60T/S88A(=GCC) | | | |
| SEQ ID NO: 88 | OXC154-S60T/S88A(=GCC) | Protein | 524 | All |
| SEQ ID NO: 89 | OXC154- | Protein | 524 | All |
| | R3T/A18E/T49R/S60T/S88A(=GCT) | | | |
| SEQ ID NO: 90 | OXC154- | Protein | 524 | All |
| | R3W/L21G/T49R/S88A(=GCC)/V97E | | | |
| SEQ ID NO: 91 | OXC154-R3T/A18E/T49R/S60T | Protein | 524 | All |
| SEQ ID NO: 92 | OXC154-P2W/T26A/S60T | Protein | 524 | All |
| SEQ ID NO: 93 | OXC154- | Protein | 524 | All |
| | R3G(=GGG)/L21G/S60T/S88A(=GCC)/V97E | | | |
| SEQ ID NO: 94 | OXC154- | Protein | 524 | All |
| | R3G(=GGG)/A18E/T49R/S88A(=GCC) | | | |
| SEQ ID NO: 95 | OXC154- | Protein | 524 | All |
| | R3T/L21G/S60T/S88A(=GCC)/V97D | | | |
| SEQ ID NO: 96 | OXC154- | Protein | 524 | All |
| | P2W/L21G/T49R/S88A(=GCC)/V97E | | | |
| SEQ ID NO: 97 | OXC154- | Protein | 524 | All |
| | R3G(=GGG)/L21G/T49R/S88A(=GCT) | | | |
| SEQ ID NO: 98 | OXC154-S295S(=TCA) | Protein | 524 | All |
| SEQ ID NO: 99 | OXC154- | Protein | 524 | All |
| | R3V/L21G/S60T/S88A(=GCC) | | | |
| SEQ ID NO: 100 | OXC154-R3T/A18E/S88A(=GCC) | Protein | 524 | All |
| SEQ ID NO: 101 | OXC154-S60T/S88A(=GCT) | Protein | 524 | All |
| SEQ ID NO: 102 | OXC154-R3W/T49R/S88A(=GCT) | Protein | 524 | All |
| SEQ ID NO: 103 | OXC154-T49R/S88A(=GCC) | Protein | 524 | All |
| SEQ ID NO: 104 | OXC154-R3W/S47F | Protein | 524 | All |
| SEQ ID NO: 105 | OXC154-A347G/I372L/L451G | Protein | 524 | All |
| SEQ ID NO: 106 | OXC154-R3G(=GGG)/L21G/S60T | Protein | 524 | All |
| SEQ ID NO: 107 | OXC154- | Protein | 524 | All |
| | R3T/L21G/T49R/S88A(=GCT) | | | |
| SEQ ID NO: 108 | OXC154-R3T/L21G/S60T | Protein | 524 | All |
| SEQ ID NO: 109 | OXC154-R3W/L21G/S88A(=GCT) | Protein | 524 | All |
| SEQ ID NO: 110 | OXC154- | Protein | 524 | All |
| | L21G/T49R/S60T/S88A(=GCT) | | | |
| SEQ ID NO: 111 | OXC154-A347G/A383V | Protein | 524 | All |
| SEQ ID NO: 112 | OXC154- | Protein | 524 | All |
| | R3W/L21G/T49R/S60T/S88A(=GCT) | | | |
| SEQ ID NO: 113 | OXC154-A18E/S88A(=GCC) | Protein | 524 | All |
| SEQ ID NO: 114 | OXC154-R3W/L21G/T49R | Protein | 524 | All |
| SEQ ID NO: 115 | OXC154-A347G/L451G | Protein | 524 | All |
| SEQ ID NO: 116 | OXC154-A347G/I372L/A383V/L451G | Protein | 524 | All |
| SEQ ID NO: 117 | OXC154-I372L/A383V/L451G | Protein | 524 | All |
| SEQ ID NO: 118 | OXC154-R3V/T49R/S88A(=GCT) | Protein | 524 | All |
| SEQ ID NO: 119 | OXC154-R3G(=GGG)/A18E/S60T | Protein | 524 | All |
| SEQ ID NO: 120 | OXC154-A347G/I372L/A383V | Protein | 524 | All |
| SEQ ID NO: 121 | OXC154-R3T | Protein | 524 | All |
| SEQ ID NO: 122 | OXC154-R3V/A18E/T49R/V97E | Protein | 524 | All |
| SEQ ID NO: 123 | OXC154- | Protein | 524 | All |
| | R3T/L21G/T49R/S60T/S88A(=GCT) | | | |
| SEQ ID NO: 124 | OXC154-R3T/L21G/T49R/V97E | Protein | 524 | All |
| SEQ ID NO: 125 | OXC154-R3V/L21G/T49R/S60T | Protein | 524 | All |
| SEQ ID NO: 126 | OXC154-G351I/I372L | Protein | 524 | All |
| SEQ ID NO: 127 | OXC154-G351I/A383V/L451G | Protein | 524 | All |
| SEQ ID NO: 128 | OXC154-G351R/I372L/L451G | Protein | 524 | All |
| SEQ ID NO: 129 | OXC154-G351I/I372L/A383V/L451G | Protein | 524 | All |
| SEQ ID NO: 130 | OXC154-G351R/I372L/A383V/L451G | Protein | 524 | All |
| SEQ ID NO: 131 | OXC154-G351I/I372L/A383V | Protein | 524 | All |
| SEQ ID NO: 132 | OXC154-N331G/Q349G/I372L/L451G | Protein | 524 | All |
| SEQ ID NO: 133 | OXC154-G351R/A383V/L451G | Protein | 524 | All |
| SEQ ID NO: 134 | OXC154-Q349G/A383V/L451G | Protein | 524 | All |
| SEQ ID NO: 135 | OXC154-A383V/L451G | Protein | 524 | All |
| SEQ ID NO: 136 | OXC154-N331G/Q349G | Protein | 524 | All |
| SEQ ID NO: 137 | OXC154-G351I | Protein | 524 | All |
| SEQ ID NO: 138 | OXC154-L451G | Protein | 524 | All |
| SEQ ID NO: 139 | OXC154-N331G/G351I/I372L/A383V | Protein | 524 | All |
| SEQ ID NO: 140 | OXC52 | Protein | 523 | All |
| SEQ ID NO: 141 | OXC154 | Protein | 524 | All |
| SEQ ID NO: 142 | NpgA | DNA | 3564 | 1170-2201 |
| SEQ ID NO: 143 | DiPKS-1 | DNA | 11114 | 849-10292 |
| SEQ ID NO: 144 | DiPKS-2 | DNA | 10890 | 717-10160 |
| SEQ ID NO: 145 | DiPKS-3 | DNA | 11300 | 795-10238 |
| SEQ ID NO: 146 | DiPKS-4 | DNA | 11140 | 794-10237 |
| SEQ ID NO: 147 | DiPKS-5 | DNA | 11637 | 1172-10615 |
| SEQ ID NO: 148 | PDH | DNA | 7114 | Ald6: 1444-2949 |
| | | | | ACS: 3888-5843 |
| SEQ ID NO: 149 | Maf1 | DNA | 3256 | 936-2123 |
| SEQ ID NO: 150 | Erg20K197E | DNA | 4254/(4538) | 2842-3900 |
| SEQ ID NO: 151 | Erg1p: UBI4-Erg20: deg | DNA | 3503 | 1364-2701 |
| SEQ ID NO: 152 | tHMGr-IDI | DNA | 4843/(4859) | tHMGRI: 885-2393 |
| | | | | IDI1: 3209-4075 |
| SEQ ID NO: 153 | PGK1p: ACC1^{S659A, S1157A} | DNA | 7673 | Pgk1p: 222-971 |
| | | | | Acc1mut: 972-7673 |
| SEQ ID NO: 154 | OAC | DNA | 2177 | 842-1150 |
| SEQ ID NO: 155 | PT254-R2S | DNA | 4707 | 1957-2925 |
| SEQ ID NO: 156 | Ostl-pro-alpha-f(I) | Protein | 92 | all |
| SEQ ID NO: 207 | OXC154 Mutant/Variant | Protein | 524 | all |
| | (generalized) | | | |
| SEQ ID NO: 206 | His tag | Protein | 6 | all |

Modifications to base strains used herein are outlined below in Table 6.

TABLE 6
Modifications to Base Strains

| | | | Integration | | Genetic |
|---|---|---|---|---|---|
| | Modification | SEQ ID | Region/ | | Structure of |
| # | name | NO: | Plasmid | Description | Sequence |
| 1 | NpgA | 142 | Flagfeldt | Phosphopantetheinyl Transferase | Site14Up::Tef1p: |
| | | | Site 14 | from Aspergillus niger. Accessory | NpgA: Prm9t: Site14Down |
| | | | integration | Protein for DiPKS (Kim et al., | |
| | | | | 2015) | |
| 2 | DiPKS-1 | 143 | USER Site | Type 1 FAS fused to Type 3 PKS | XII- |
| | | | XII-1 | from D. discoideum. Produces | 1up::Gal1p: DiPKSG1516R: |
| | | | integration | Olivetol from malonyl-coA | Prm9t::XII1-down |
| | | | (Jensen et | | |
| | | | al, 2014) | | |
| 3 | DiPKS-2 | 144 | Wu site 1 | Type 1 FAS fused to Type 3 PKS | Wu1up::Gal1p: |
| | | | integration | from D. discoideum. Produces | DiPKSG1516R: |
| | | | | Olivetol from malonyl-coA | Prm9t::Wu1down |
| 4 | DiPKS-3 | 145 | Wu site 3 | Type 1 FAS fused to Type 3 PKS | Wu3up::Gal1p: |
| | | | integration | from D. discoideum. Produces | DiPKSG1516R: |
| | | | | Olivetol from malonyl-coA | Prm9t::Wu3down |
| 5 | DiPKS-4 | 146 | Wu site 6 | Type 1 FAS fused to Type 3 PKS | Wu6up::Gal1p: |
| | | | integration | from D. discoideum. Produces | DiPKSG1516R: |
| | | | | Olivetol from malonyl-coA | Prm9t::Wu6down |
| 6 | DiPKS-5 | 147 | Wu site 18 | Type 1 FAS fused to Type 3 PKS | Wu18up::Gal1p: |
| | | | integration | from D. discoideum. Produces | DiPKSG1516R: |
| | | | | Olivetol from malonyl-coA | Prm9t::Wu18down |
| 7 | PDH | 148 | Flagfeldt | Acetaldehyde dehydrogenase | 19Up::Tdh3p: Ald6: |
| | | | Site 19 | (ALD6) from S. cerevisiae and | Adh1::Tef1p: seACS1^{L641p}: Prm9t::19Down |
| | | | integration | acetoacetyl coA synthase | |
| | | | | (AscL641P) from Salmonella | |
| | | | | enterica. Will allow greater | |
| | | | | accumulation of acetyl-coA in the | |
| | | | | cell. (Shiba et al., 2007) | |
| 8 | Maf1 | 149 | Flagfeldt | Maf1 is a regulator of tRNA | Site5Up::Tef1p: Maf1: |
| | | | Site 5 | biosynthesis. Overexpression in | Prm9t: Site5Down |
| | | | integration | S. cerevisiae has demonstrated | |
| | | | | higher monoterpene (GPP) yields | |
| | | | | (Liu et al., 2013) | |
| 9 | Erg20K197E | 150 | Chromosomal | Mutant of Erg20 protein that | Tpi1t: ERG20K197E: |
| | | | modification | diminishes FPP synthase activity | Cyc1t::Tef1p: KanMX: Tef1t |
| | | | | creating greater pool of GPP | |
| | | | | precursor. Negatively affects | |
| | | | | growth phenotype (Oswald et al., | |
| | | | | 2007) | |
| 10 | Erg1p: UBI4- | SEQ. | Flagfeldt | Sterol responsive promoter | Site18Up::Erg1p: |
| | Erg20: deg | 151 | Site 18 | controlling Erg20 protein activity. | UBI4deg: ERG20: Adh1t: |
| | | | integration | Allows for regular FPP synthase | Site18down |
| | | | | activity and uninhibited growth | |
| | | | | phenotype until accumulation of | |
| | | | | sterols which leads to a | |
| | | | | suppression of expression of | |
| | | | | enzyme. (Peng et al., 2018) | |
| 11 | tHMGr- | 152 | USER Site | Overexpression of truncated | X3up::Tdh3p: tHMGR1: |
| | IDI | | X-3 | HMGr1 and IDI1 proteins that | Adh1t::Tef1p: IDI1: |
| | | | integration | have been previously identified to | Prm9t::X3down |
| | | | | be bottlenecks in the | |
| | | | | S. cerevisiae terpenoid pathway | |
| | | | | responsible for GPP production | |
| | | | | (Ro et al., 2006) | |
| 12 | PGK1p: | 153 | Chromosomal | Mutations in the native | Pgk1: ACC1^{S659A, S1157A}: |
| | ACC1^{S659A, S1157A} | | modification | S. cerevisiae acetyl-coA | Acc1t |
| | | | | carboxylase that removes post- | |
| | | | | translational modification based | |
| | | | | down-regulation. Leads to greater | |
| | | | | malonyl-coA pools. The promoter | |
| | | | | of Acc1 was also changed to a | |
| | | | | constitutive promoter for higher | |
| | | | | expression (Shi et al., 2014) | |
| 13 | OAC | 154 | Flagfeldt | The Cannabis sativa Olivetolic | FgF16up::Gal1p: csOAC: |
| | | | Site 16 | acid cyclase (OAC) protein allows | Eno2t::FgF16down |
| | | | integration | the production of olivetolic acid | |
| | | | | from a polyketide precursor. | |
| 14 | PT254- | 155 | Flagfeldt | The Cannabis sativa | FgF18up::Tef1p: |
| | R2S | | Site 18 | prenyltransferase PT254 allows | R2S-PT254: Cyct::FgF20down |
| | | | integration | CBGa to be produced from | |
| | | | | olivetolic acid and geranyl | |
| | | | | pyrophosphate (Luo et al., 2019). | |
| | | | | The N terminal arginine of this | |
| | | | | enzyme has been replaced with a | |
| | | | | serine in order to enhance protein | |
| | | | | stability in accordance with N-end | |
| | | | | rule (Varshavsky 1996). | |

Results:

Production of Cannabidiolic Acid

An OXC154 variant library was constructed in a plasmid regulated by the Gal1p promoter, and expressed in a CBGa-producing background strain (HB965) harbouring upstream enzymes of the cannabinoid production pathway. Strains expressing wild type OXC154 (HB21) and mScarlet fluorescent non-catalytic protein (HB1741) were utilized as controls in the screening to facilitate identification of OXC154 variants with improved activity.

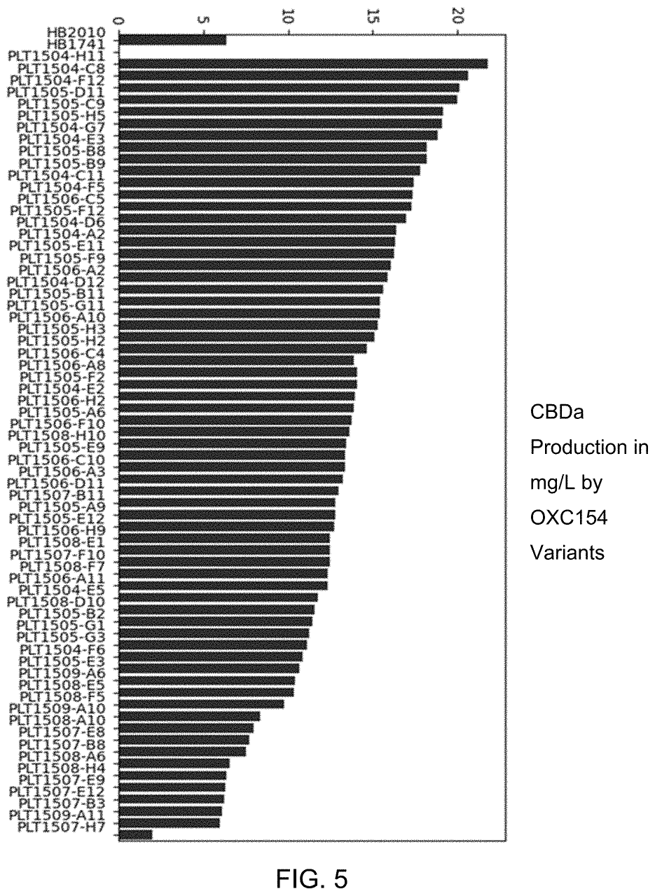

FIG. 5 shows cannabinoid CBDa production by engineered DXC154 variant strains. The CBDa production values (mg/I) observed for the different engineered OXC154 variant strains are shown.

Table 7 relates further information regarding cannabinoid production of the strains shown in FIG. 5. In particular, Table 7 shows production of olivetol, olivetolic acid, CBGa, THCa, CBDa, lists 00600, reports ratio of CBDa to [THCa+CBDa] combined, ratio of C+Da to [CBGa+CBDa] combined, and reports the ratio of CBDa to upstream metabolites in wild type and engineered OXC154 mutant strains.

TABLE 7
Production of CBDa, Upstream Metabolites, and other Cannabinoids

| | | | | | | | | Ratio of | Ratio of | Ratio of |
|---|---|---|---|---|---|---|---|---|---|---|
| Sample ID | | | Olivetolic | | | | | CBDa to | CBDa to | CBDa to |
| in the | | Olivetol | Acid | CBGa | THCa | CBDa | | [THCa + | [CBGa + | upstream |
| figure | Strain | (mg/L) | (mg/L) | (mg/L) | (mg/L) | (mg/L) | OD600 | CBDa] | CBDa] | metabolites |
| OXC154 | HB2010 | 57.729 | 91.475 | 7.507 | 0.493 | 6.282 | 3.422 | 0.949 | 0.53 | 0.043 |
| RFP | HB1741 | 70.825 | 67.6 | 19.1 | 0 | 0 | 3.845 | — | 0 | 0 |
| mScarlet | | | | | | | | | | |
| PLT1504- | OXC154- | 60.267 | 99.1 | 0.9 | 1.533 | 21.7 | 3.355 | 0.934 | 0.96 | 0.137 |
| H11 | R3G(=GGG)/ | | | | | | | | | |
| | L21G/S60T/ | | | | | | | | | |
| | S88A(=GCT) | | | | | | | | | |
| PLT1504- | OXC154- | 45.167 | 70.633 | 1.667 | 1.6 | 20.567 | 3.169 | 0.928 | 0.925 | 0.175 |
| C8 | R3G(=GGG)/ | | | | | | | | | |
| | A18E/T49R/S60T/ | | | | | | | | | |
| | S88A(=GCT) | | | | | | | | | |
| PLT1504- | OXC154- | 51.883 | 84.05 | 1.933 | 1.567 | 20.033 | 3.298 | 0.927 | 0.915 | 0.145 |
| F12 | R3T/T49R/ | | | | | | | | | |
| | S88A(=GCC) | | | | | | | | | |
| PLT1505- | OXC154- | 38.167 | 57.7 | 3.767 | 1.4 | 19.933 | 3.114 | 0.934 | 0.841 | 0.201 |
| D11 | R3W/A18E/T49R/ | | | | | | | | | |
| | S60T/S88A | | | | | | | | | |
| | (=GCT) | | | | | | | | | |
| PLT1505- | OXC154- | 35.233 | 58 | 2.533 | 1.433 | 19.067 | 3.325 | 0.93 | 0.883 | 0.199 |
| C9 | R3V/T49R/S60T/ | | | | | | | | | |
| | S88A(=GCT) | | | | | | | | | |
| PLT1505- | OXC154- | 46.85 | 72.533 | 2.517 | 1.467 | 19.033 | 3.461 | 0.929 | 0.88 | 0.159 |
| H5 | R3V/T49R/S60T/ | | | | | | | | | |
| | S88A(=GCC) | | | | | | | | | |
| PLT1504- | OXC154- | 60.8 | 96.933 | 4.7 | 1.333 | 18.733 | 3.403 | 0.935 | 0.798 | 0.116 |
| G7 | A18A(=GCC) | | | | | | | | | |
| PLT1504- | OXC154- | 43.6 | 74.767 | 1.733 | 1.433 | 18.133 | 3.17 | 0.927 | 0.914 | 0.152 |
| E3 | R3T/A18E/T49R/ | | | | | | | | | |
| | S88A(=GCC) | | | | | | | | | |
| PLT1505- | OXC154-R3T/ | 34.8 | 56.267 | 3.567 | 1.367 | 18.1 | 3.21 | 0.93 | 0.837 | 0.192 |
| B8 | S88A(=GCC) | | | | | | | | | |
| PLT1505- | OXC154- | 33.9 | 57.2 | 2.133 | 1.3 | 17.7 | 2.94 | 0.932 | 0.895 | 0.191 |
| B9 | R3G(=GGG)/ | | | | | | | | | |
| | L21G/T49R | | | | | | | | | |
| PLT1504- | OXC154- | 44.233 | 70.1 | 3 | 1.4 | 17.333 | 2.995 | 0.925 | 0.851 | 0.15 |
| C11 | R3T/T49R/ | | | | | | | | | |
| | S88A(=GCT) | | | | | | | | | |
| PLT1504- | OXC154- | 53.833 | 86.667 | 6.933 | 1.3 | 17.3 | 3.389 | 0.933 | 0.744 | 0.123 |
| F5 | R3G(=GGA)/ | | | | | | | | | |
| | A18E/T49R/S60T/ | | | | | | | | | |
| | S88A(=GCC) | | | | | | | | | |
| PLT1506- | OXC154-R3W/T49R/ | 47.433 | 83.2 | 2.433 | 1.233 | 17.2 | 3.44 | 0.933 | 0.877 | 0.129 |
| C5 | S88A(=GCC)/V97E | | | | | | | | | |
| PLT1505- | OXC154- | 53.267 | 90.667 | 1.1 | 1.2 | 16.867 | 3.619 | 0.934 | 0.938 | 0.116 |
| F12 | R3G(=GGG)/ | | | | | | | | | |
| | A18E/S88A(=GCC) | | | | | | | | | |
| PLT1504- | OXC154- | 42.967 | 73.433 | 1.6 | 1.3 | 16.333 | 3.09 | 0.927 | 0.905 | 0.14 |
| D6 | R3V/A18E/T49R/ | | | | | | | | | |
| | S60T/S88A(=GCC) | | | | | | | | | |
| PLT1504- | OXC154- | 47.25 | 74.95 | 3.233 | 1.25 | 16.267 | 3.362 | 0.93 | 0.83 | 0.134 |
| A2 | S60T/S88A(=GCC) | | | | | | | | | |
| PLT1505- | OXC154- | 49.9 | 85.433 | 0.433 | 1.2 | 16.167 | 3.594 | 0.932 | 0.973 | 0.121 |
| E11 | R3T/A18E/T49R/ | | | | | | | | | |
| | S60T/S88A(=GCT) | | | | | | | | | |
| PLT1505- | OXC154-R3W/ | 35.267 | 61.267 | 2.033 | 1.267 | 16 | 2.996 | 0.927 | 0.883 | 0.164 |
| F9 | L21G/T49R/ | | | | | | | | | |
| | S88A(=GCC)/ | | | | | | | | | |
| | V97E | | | | | | | | | |
| PLT1506- | OXC154- | 64.067 | 109.767 | 2.133 | 0.933 | 15.8 | 3.446 | 0.946 | 0.867 | 0.091 |
| A2 | R3T/A18E/ | | | | | | | | | |
| | T49R/S60T | | | | | | | | | |
| PLT1504- | OXC154- | 47.167 | 76.533 | 7.767 | 1.167 | 15.533 | 3.434 | 0.936 | 0.685 | 0.122 |
| D12 | P2W/T26A/S60T | | | | | | | | | |
| PLT1505- | OXC154- | 35.8 | 66.333 | 1.967 | 1.167 | 15.367 | 3.158 | 0.93 | 0.88 | 0.152 |
| B11 | R3G(=GGG)/ | | | | | | | | | |
| | L21G/S60T/ | | | | | | | | | |
| | S88A(=GCC)/ | | | | | | | | | |
| | V97E | | | | | | | | | |
| PLT1505- | OXC154- | 47.483 | 69.983 | 4.95 | 1.1 | 15.333 | 3.273 | 0.935 | 0.737 | 0.122 |
| G11 | R3G(=GGG)/A18E/ | | | | | | | | | |
| | T49R/S88A(=GCC) | | | | | | | | | |
| PLT1506- | OXC154- | 49.4 | 88.367 | 1.2 | 1.133 | 15.233 | 3.522 | 0.932 | 0.924 | 0.11 |
| A10 | R3T/L21G/S60T/ | | | | | | | | | |
| | S88A(=GCC)/ | | | | | | | | | |
| | V97D | | | | | | | | | |
| PLT1505- | OXC154- | 51.017 | 89.717 | 2.217 | 1.15 | 15.033 | 3.498 | 0.93 | 0.867 | 0.105 |
| H3 | P2W/L21G/T49R/ | | | | | | | | | |
| | S88A(=GCC)/ | | | | | | | | | |
| | V97E | | | | | | | | | |
| PLT1505- | OXC154- | 50.6 | 84.4 | 3.1 | 1.033 | 14.567 | 3.511 | 0.934 | 0.813 | 0.108 |
| H2 | R3G(=GGG)/ | | | | | | | | | |
| | L21G/T49R/ | | | | | | | | | |
| | S88A(=GCT) | | | | | | | | | |
| PLT1506- | OXC154- | 47.933 | 86.617 | 1.117 | 1.05 | 14.267 | 3.466 | 0.932 | 0.924 | 0.105 |
| C4 | S295S(=TCA) | | | | | | | | | |
| PLT1506- | OXC154- | 39.6 | 66.333 | 0.967 | 0.933 | 14 | 3.279 | 0.938 | 0.934 | 0.132 |
| A8 | R3V/L21G/S60T/ | | | | | | | | | |
| | S88A(=GCC) | | | | | | | | | |
| PLT1505- | OXC154- | 54.733 | 91.767 | 2.133 | 1.067 | 14 | 3.304 | 0.931 | 0.863 | 0.092 |
| F2 | R3T/A18E/ | | | | | | | | | |
| | S88A(=GCC) | | | | | | | | | |
| PLT1504- | OXC154-S60T/ | 49.833 | 88.733 | 2.833 | 1.033 | 13.9 | 3.045 | 0.932 | 0.824 | 0.097 |
| E2 | S88A(=GCT) | | | | | | | | | |
| PLT1506- | OXC154- | 49.767 | 88.367 | 2.583 | 1 | 13.8 | 3.437 | 0.934 | 0.833 | 0.103 |
| H2 | R3W/T49R/ | | | | | | | | | |
| | S88A(=GCT) | | | | | | | | | |
| PLT1505- | OXC154-T49R/ | 46.5 | 73.833 | 2.167 | 0.833 | 13.7 | 3.112 | 0.951 | 0.86 | 0.123 |
| A6 | S88A(=GCC) | | | | | | | | | |
| PLT1506- | OXC154- | 48.733 | 86.433 | 2.833 | 0.967 | 13.567 | 3.446 | 0.933 | 0.832 | 0.102 |
| F10 | R3W/S47F | | | | | | | | | |
| PLT1508- | OXC154- | 48.856 | 82.156 | 2.167 | 1.1 | 13.333 | 3.458 | 0.925 | 0.854 | 0.101 |
| H10 | A347G/I372L/ | | | | | | | | | |
| | L451G | | | | | | | | | |
| PLT1505- | OXC154- | 49.133 | 85.4 | 4.633 | 0.867 | 13.3 | 3.389 | 0.941 | 0.748 | 0.096 |
| E9 | R3G(=GGG)/ | | | | | | | | | |
| | L21G/S60T | | | | | | | | | |
| PLT1506- | OXC154- | 44.433 | 82.6 | 1.9 | 0.9 | 13.3 | 3.5 | 0.937 | 0.867 | 0.104 |
| C10 | R3T/L21G/T49R/ | | | | | | | | | |
| | S88A(=GCT) | | | | | | | | | |
| PLT1506- | OXC154- | 46.467 | 84.867 | 1.033 | 0.967 | 13.3 | 3.41 | 0.932 | 0.925 | 0.101 |
| C4 | S295S(=TCA) | | | | | | | | | |
| PLT1506- | OXC154- | 51.367 | 88.85 | 1.85 | 0.967 | 13.2 | 3.44 | 0.932 | 0.873 | 0.093 |
| A3 | R3W/L21G/ | | | | | | | | | |
| | S88A(=GCT) | | | | | | | | | |
| PLT1506- | OXC154- | 42.4 | 78.389 | 1.456 | 0.967 | 12.944 | 3.351 | 0.931 | 0.893 | 0.106 |
| D11 | L21G/T49R/S60T/ | | | | | | | | | |
| | S88A(=GCT) | | | | | | | | | |
| PLT1507- | OXC154- | 43.667 | 68.7 | 8.467 | 0.933 | 12.733 | 3.437 | 0.932 | 0.599 | 0.105 |
| B11 | A347G/A383V | | | | | | | | | |
| PLT1505- | OXC154- | 48.133 | 86.467 | 1.367 | 0.867 | 12.7 | 3.307 | 0.936 | 0.904 | 0.094 |
| A9 | R3W/L21G/T49R/ | | | | | | | | | |
| | S60T/S88A(=GCT) | | | | | | | | | |
| PLT1505- | OXC154-A18E/ | 50.367 | 90.933 | 3.4 | 0.9 | 12.667 | 3.382 | 0.935 | 0.774 | 0.087 |
| E12 | S88A(=GCC) | | | | | | | | | |
| PLT1506- | OXC154- | 42.633 | 77.233 | 2.467 | 0.8 | 12.4 | 3.439 | 0.939 | 0.832 | 0.103 |
| H9 | R3W/L21G/T49R | | | | | | | | | |
| PLT1508- | OXC154- | 49.933 | 83.3 | 3.267 | 0.967 | 12.4 | 3.405 | 0.927 | 0.783 | 0.093 |
| E1 | A347G/L451G | | | | | | | | | |
| PLT1507- | OXC154- | 45.733 | 74.717 | 1.967 | 1.033 | 12.383 | 3.511 | 0.923 | 0.862 | 0.102 |
| F10 | A347G/I372L/ | | | | | | | | | |
| | A383V/L451G | | | | | | | | | |
| PLT1508- | OXC154- | 43.2 | 71 | 4.567 | 0.833 | 12.267 | 3.508 | 0.937 | 0.732 | 0.103 |
| F7 | I372L/A383V/L451G | | | | | | | | | |
| PLT1506- | OXC154- | 45.333 | 83.1 | 1.467 | 0.8 | 12.267 | 3.135 | 0.939 | 0.893 | 0.095 |
| A11 | R3V/T49R/ | | | | | | | | | |
| | S88A(=GCT) | | | | | | | | | |
| PLT1504- | OXC154- | 45.2 | 74.2 | 10.833 | 0.7 | 11.667 | 3.276 | 0.944 | 0.513 | 0.089 |
| E5 | R3G(=GGG)/ | | | | | | | | | |
| | A18E/S60T | | | | | | | | | |
| PLT1508- | OXC154- | 48.733 | 89.3 | 3.6 | 0.8 | 11.533 | 3.486 | 0.934 | 0.752 | 0.081 |
| D10 | A347G/I372L/A383V | | | | | | | | | |
| PLT1505- | OXC154-R3T | 65.925 | 65.55 | 0.175 | 0.65 | 11.4 | 3.832 | 0.952 | 0.977 | 0.086 |
| B2 | | | | | | | | | | |
| PLT1505- | OXC154-R3V/ | 34.1 | 57.933 | 8.933 | 0.733 | 11.167 | 3.039 | 0.938 | 0.557 | 0.11 |
| G1 | A18E/T49R/V97E | | | | | | | | | |
| PLT1505- | OXC154- | 34.467 | 62.333 | 7.567 | 0.7 | 11.033 | 3.216 | 0.945 | 0.598 | 0.105 |
| G3 | R3T/L21G/T49R/ | | | | | | | | | |
| | S60T/S88A(=GCT) | | | | | | | | | |
| PLT1504- | OXC154- | 44.267 | 73.033 | 11.633 | 0.667 | 10.8 | 3.106 | 0.942 | 0.478 | 0.085 |
| F6 | R3T/L21G/T49R/ | | | | | | | | | |
| | V97E | | | | | | | | | |
| PLT1505- | OXC154- | 43.333 | 77.667 | 1.567 | 0.75 | 10.617 | 3.307 | 0.945 | 0.799 | 0.082 |
| E3 | R3V/L21G/T49R/ | | | | | | | | | |
| | S60T | | | | | | | | | |
| PLT1509- | OXC154- | 51.6 | 85.6 | 6.2 | 0.6 | 10.333 | 3.723 | 0.945 | 0.626 | 0.073 |
| A6 | G351I/I372L | | | | | | | | | |
| PLT1508- | OXC154- | 48.767 | 86.433 | 4.167 | 0.8 | 10.267 | 3.531 | 0.928 | 0.713 | 0.074 |
| E5 | G351I/A383V/ | | | | | | | | | |
| | L451G | | | | | | | | | |
| PLT1508- | OXC154- | 70.7 | 50.15 | 1.325 | 0 | 9.675 | 3.948 | 1 | 0.863 | 0.08 |
| F5 | G351R/I372L/ | | | | | | | | | |
| | L451G | | | | | | | | | |
| PLT1509- | OXC154- | 71.683 | 54.975 | 2.05 | 0.067 | 8.283 | 3.626 | 0.994 | 0.8 | 0.064 |
| A10 | G351I/I372L/ | | | | | | | | | |
| | A383V/L451G | | | | | | | | | |
| PLT1508- | OXC154- | 66.34 | 51.085 | 2.87 | 0.175 | 7.875 | 3.676 | 0.982 | 0.729 | 0.064 |
| A10 | G351R/I372L/ | | | | | | | | | |
| | A383V/L451G | | | | | | | | | |
| PLT1507- | OXC154- | 71.125 | 61.175 | 4.45 | 0 | 7.675 | 3.621 | 1 | 0.635 | 0.056 |
| E8 | G3511/I372L/ | | | | | | | | | |
| | A383V | | | | | | | | | |
| PLT1507- | OXC154- | 65.525 | 57.525 | 5.4 | 0 | 7.45 | 3.642 | 1 | 0.575 | 0.058 |
| B8 | N331G/Q349G/ | | | | | | | | | |
| | I372L/L451G | | | | | | | | | |
| PLT1508- | OXC154-G351R/ | 66.7 | 52.975 | 4.6 | 0 | 6.475 | 3.875 | 1 | 0.583 | 0.052 |
| A6 | A383V/L451G | | | | | | | | | |
| PLT1508- | OXC154-Q349G/ | 72.6 | 48.375 | 4.975 | 0 | 6.325 | 3.885 | 1 | 0.555 | 0.05 |
| H4 | A383V/L451G | | | | | | | | | |
| PLT1507- | OXC154- | 66.1 | 55.375 | 6.175 | 0 | 6.25 | 3.752 | 1 | 0.492 | 0.049 |
| E9 | A383V/L451G | | | | | | | | | |
| PLT1507- | OXC154- | 73.375 | 63.25 | 7.375 | 0 | 6.2 | 3.847 | 1 | 0.459 | 0.043 |
| E12 | N331G/Q349G | | | | | | | | | |
| PLT1507- | OXC154- | 70.375 | 59.625 | 7.45 | 0 | 6.025 | 3.825 | 1 | 0.443 | 0.043 |
| B3 | G351I | | | | | | | | | |
| PLT1509- | OXC154- | 76.2 | 56.45 | 7.1 | 0 | 5.925 | 3.513 | 1 | 0.469 | 0.042 |
| A11 | L451G | | | | | | | | | |
| PLT1507- | OXC154- | 48.35 | 41.8 | 8.35 | 0 | 1.925 | 3.694 | 1 | 0.155 | 0.02 |
| H7 | N331G/G351I/ | | | | | | | | | |
| | I372L/A383V | | | | | | | | | |

Table 8 provides a summary of mutations described herein, with additional mutations being described in Table 15, below.

TABLE 8
Mutations

| | | Number of occurrences |
|---|---|---|
| | | in SEQ ID NO: 72 to |
| Mutation | Type | SEQ ID NO: 139 |
| P2W | Non-conservative | 2 |
| R3T | Non-conservative | 12 |
| R3G(=GGG) | Non-conservative | 9 |
| R3G(=GGA) | Non-conservative | 1 |
| R3W | Non-conservative | 8 |
| R3V | Non-conservative | 7 |
| A18E | Non-conservative | 13 |
| A18A(=GCC) | Non-conservative | 1 |
| L21G | Conservative | 17 |
| T26A | Non-conservative | 1 |
| N331G | Non-conservative | 3 |
| S47F | Non-conservative | 1 |
| T49R | Non-conservative | 28 |
| S60T | Conservative | 21 |
| S88A(=GCC) | Non-conservative | 18 |
| S88A(=GCT) | Non-conservative | 15 |
| V97E | Non-conservative | 6 |
| V97D | Non-conservative | 1 |
| S295S(=TCA) | Conservative | 2 |
| A347G | Conservative | 5 |
| Q349G | Non-conservative | 3 |
| G351I | Conservative | 6 |
| G351R | Non-conservative | 3 |
| I372L | Conservative | 11 |
| A383V | Conservative | 12 |
| L451G | Conservative | 13 |

Use in Host Cells

Phytocannabinoids, such as tetrahydrocannabinol (THC) and cannabidiol (CEO), can be extracted from plant material for medical and psychotropic purposes. However, the synthesis of plant material is costly, not readily scalable to large volumes, and requires lengthy growth periods to produce sufficient quantities of phytocannabinoids. An organism capable of fermentation, such as Saccharomyces cerevisiae, that is capable of producing cannabinoids would provide an economical route to producing these compounds on an industrial scale.

The early stages of the cannabinoid pathway proceeds via the generation of olivetolic acid by the type III PKS olivetolic acid synthase (OAS) and cyclase olivetolic acid cyclase (OAC). This reaction uses a hexanoyl-CoA starter as well as three units of malonyl-CoA. Olivetolic acid is the backbone of most classical cannabinoids and can be prenylated to form CBGA, which is ultimately converted to CBDA or THCA by an oxidocyclase. Downstream phytocannabinoids can be prepared therefrom, and CBDa synthase activity based on the OXC154 variants described herein is envisioned for use in host cells.

Table 9 lists specific examples of host cell organisms in which the described cannabidiolic acid synthase (CBDa synthase) OXC154 variants may be utilized for preparation of cannabinoids in the described pathways.

TABLE 9
List of Host Cell Organisms

| Type | Organisms |
|---|---|
| Bacteria | Escherichia coli, Streptomyces coelicolor and other species., Bacillus |
| | subtilis, Mycoplasma genitalium, Synechocytis, Zymomonas mobilis, |
| | Corynebacterium glutamicum, Synechococcus sp., Salmonella typhi, |
| | Shigella flexneri, Shigella sonnei, and Shigella disenteriae, Pseudomonas |
| | putida, Pseudomonas aeruginosa, Pseudomonas mevalonii, Rhodobacter |
| | sphaeroides, Rhodobacter capsulatus, Rhodospirillum rubrum, |
| | Rhodococcus sp. |
| Fungi | Saccharomyces cerevisiae, Ogataea polymorpha, Komagataella phaffii, |
| | Kluyveromyces lactis, Neurospora crassa, Aspergillus niger, Aspergillus |
| | nidulans, Schizosaccharomyces pombe, Yarrowia lipolytica, |
| | Myceliophthora thermophila, Aspergillus oryzae, Trichoderma reesei, |
| | Chrysosporium lucknowense, Fusarium sp., Fusarium gramineum, |
| | Fusarium venenatum, Pichia finlandica, Pichia trehalophila, Pichia |
| | koclamae, Pichia membranaefaciens, Pichia opuntiae, Pichia |
| | thermotolerans, Pichia salictaria, Pichia guercuum, Pichia pijperi, Pichia |
| | stipitis, Pichia methanolica, Hansenula polymorpha. |
| Protists | Chlamydomonas reinhardtii, Dictyostelium discoideum, Chlorella sp., |
| | Haematococcus pluvialis, Arthrospira platensis, Dunaliella sp., |
| | Nannochloropsis oceanica. |
| Plants | Cannabis sativa, Arabidopsis thaliana, Theobroma cacao, maize, banana, |
| | peanut, field peas, sunflower, Nicotiana sp., tomato, canola, wheat, barley, |
| | oats, potato, soybeans, cotton, sorghum, lupin, rice. |

Phytocannabinoids may be produced in a host cell involving Dictyostelium discoideum polyketide synthase (DiPKS), olivetolic acid cyclase (OAC), prenyltransferases, and/or mutants of these, as described in Applicant's co-pending International Application No: PCT/CA2020/050687 (herein incorporated by reference). For example, a host cell transformed with a polyketide synthase coding sequence, an olivetolic acid cyclase coding sequence, and a prenyltransferase coding sequence may be prepared. The polyketide synthase and the olivetolic acid cyclase catalyze synthesis of olivetolic acid from malonyl CoA. The cannabidiolic acid (CBDa) synthase may include any of the functional mutants described herein. The host cell may include a yeast cell, a bacterial cell, a protest cell or a plant cell, selected from among those listed in Table 9.

Combinations of the methods, nucleotides, and expression vectors described herein as well as in Applicant's co-pending International Application No: PCT/CA2020/050687 may be employed together to produce CBDa, as well as other phytocannabinoids and phytocannabinoid precursors. Depending on the desired product, selections of characteristics of the cells and methods employed may be selected to achieve production of the cannabinoid, cannabinoid precursor, or intermediate of interest. For example, cannabivarins may be produced.

Methods of producing a phytocannabinoid may comprising culturing a host cell under suitable culture conditions to form a phytocannabinoid, said host cell comprising: a polynucleotide encoding a polyketide synthase (PKS) enzyme; a polynucleotide encoding an olivetolic acid cyclase (OAC) enzyme mutants as described herein; and a polynucleotide encoding a prenyltransferase (PT) enzyme; and optionally comprising: a polynucleotide encoding an acyl-CoA synthetase (Alk) enzyme; a polynucleotide encoding a fatty acyl CoA activating (CsAAE) enzyme; and/or a polynucleotide encoding a THCa synthase (OXC) enzyme.

An expression vector can be prepared comprising a polynucleotide encoding a polyketide synthase (PKS) enzyme; a polynucleotide encoding an olivetolic acid cyclase (OAC) enzyme mutants as described herein; and a polynucleotide encoding a prenyltransferase (PT) enzyme. The expression vector can optionally comprise a polynucleotide encoding an acyl-CoA synthetase (Alk) enzyme; a polynucleotide encoding an acyl-activating enzyme CsAAE1; and/or a polynucleotide encoding a THCa synthase (OXC) enzyme.

### Example 2

Set of CBDa Producing OXC Mutants Derived from OXC161

OXC161 is an OXC154 mutant as described in Example 1 (SEQ ID NO:59 (DNA) and SEQ ID NO:127 (AA)). Wild type cannabidiolic acid synthase (CBDa synthase), having been modified with the insertion of a serine between positions 224 and 225 in the OXC52 sequence, results in OXC154, a modified cannabidiolic acid synthase with improved CBDa production as compared with OXC52. OXC154 is described in Applicant's publication WO202/0232553 (PCT application PCT/CA2020/050687). Variants of OXC154, termed “OXC161”, and its mutants having CBDa synthase activity are prepared.

Materials and Methods:

Genetic Manipulations in the directed evolution of OXC. Genetic manipulations where conducted according to the same methodology as in Example 1, with the exception that OXC161 (SEQ ID NO:59) was used as the template plasmid for mutagenesis in place of OXC154. Modifications made to base strains for Examples 2-5 are outlined in Table 14, below, and point substitutions described in Examples 2-4, wherein amino acid position numbers refer to the OXC154 sequence, are provided in Table 15 below.

Strain growth and Media. Same as in Example 1.

Quantification Protocol. Same as in Example 1.

Results:

Production of Cannabidiolic Acid. Clonal strains harboring a library of mutant OXC161 (OXC154-G351 l/A383V/L451G) variants were expressed on plasmids under control of pGAL1 in a CBGa-producing background (HB2191). Comparison with strains expressing OXC161(HB2522) and a non-catalytic control mScarlet (HB2523) facilitated the identification of novel OXC variants with improved activity.

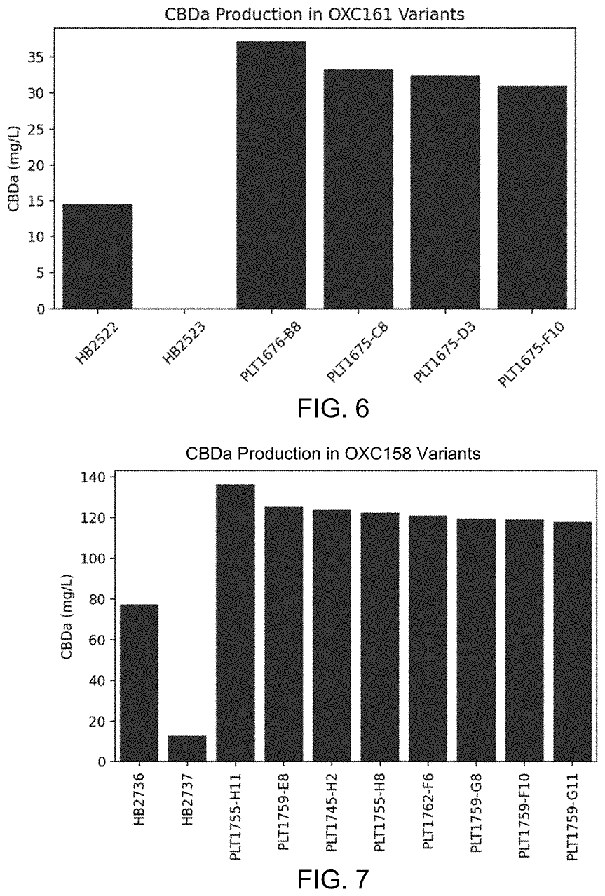

FIG. 6 shows cannabinoid production values in strains containing expressing OXC161 variants identified through a combinatorial library.

Table 10 shows production of C:Da and upstream metabolites observed in this example.

TABLE 10
Production of CBDa and Upstream Metabolites

| | | | | | | | | Ratio of | Ratio of | Ratio of |
|---|---|---|---|---|---|---|---|---|---|---|
| Sample ID | | | Olivetolic | | | | | CBDa to | CBDa to | CBDa to |
| in the | | Olivetol | Acid | CBGa | THCa | CBDa | | (THCa + | (CBGa + | upstream |
| figure | Strain | (mg/L) | (mg/L) | (mg/L) | (mg/L) | (mg/L) | OD600 | CBDa) | CBDa) | metabolites |
| HB2522 | OXC154- | 18.667 | 54.833 | 40.500 | 4.500 | 14.500 | 3.368 | 0.763 | 0.265 | 0.088 |
| | G351I/A383V/ | | | | | | | | | |
| | L451G | | | | | | | | | |
| HB2523 | mScarlet | 18.500 | 44.667 | 67.667 | 0.000 | 0.000 | 3.256 | | 0.000 | 0.000 |
| PLT1676- | OXC154- | 29.833 | 92.000 | 30.000 | 7.500 | 37.167 | 3.720 | 0.832 | 0.555 | 0.157 |
| B8 | R3G/A18E/S60T/ | | | | | | | | | |
| | G351I/A383V/ | | | | | | | | | |
| | L451G | | | | | | | | | |
| PLT1675- | OXC158 (OXC154- | 22.167 | 63.500 | 31.500 | 8.667 | 33.333 | 3.381 | 0.794 | 0.516 | 0.189 |
| C8 | R3W/A18E/T49R/ | | | | | | | | | |
| | V97E/G351I/A383V/ | | | | | | | | | |
| | L451G) | | | | | | | | | |
| PLT1675- | OXC154- | 23.167 | 86.667 | 27.167 | 7.167 | 32.500 | 3.432 | 0.819 | 0.546 | 0.154 |
| D3 | R3W/A18E/T49R/ | | | | | | | | | |
| | V97E/G351I/A383V/ | | | | | | | | | |
| | L451G | | | | | | | | | |
| PLT1675- | OXC154- | 17.667 | 60.167 | 8.000 | 8.500 | 31.000 | 3.357 | 0.785 | 0.796 | 0.223 |
| F10 | R3T/S60T/G351I/ | | | | | | | | | |
| | A383V/L451G | | | | | | | | | |

### Example 3

Set of CBDa Producing OXC Mutants Derived from OXC158

Wild type cannabidiolic acid synthase (OXC52 of 523 amino acids in length, represented herein as SEQ ID NO:140), when modified with the insertion of a serine between positions 224 and 225 is referred to herein as OXC154 (OXC154 being 524 amino acids in length, as represented here in as SEQ ID NO:141). In Example 2, OXC161 is formed, as derived from OXC154. In this example, OXC158 is formed as an OXC161 mutant. OXC158 may be referenced herein interchangeably with SEQ ID NO:162 (protein), and noting that SEQ ID NO:158 represents the DNA therefor, which may also be referenced as OXC154-R3W/A18E/T49R/V97E/G351I/A383V/L451G, representing the substitutions relative to the amino acids of OXC154 (with OXC154 being represented herein as SEQ ID:141). CBDa producing cannabidiolic acid synthase mutants of OXC158 are described with reference to the substitution positions relative to OXC154 (SEQ ID NO:141), or relative to OXC158 (SEQ ID NO:162), if so specified.

Materials and Methods:

Genetic Manipulations. Site-saturation mutagenesis libraries were constructed using the same kinase-ligase-Dpnl methods described in Example 1. OXC158 was used as the parental template sequence. Plasmid transformations into yeast cells were performed as described in Example 1. Modifications made to base strains for Examples 2-5 are outlined in Table 14, below, and point substitutions described in Examples 2-4, wherein amino acid position numbers refer to the OXC154 sequence, are provided in Table 15 below.

Strain Growth and Media. Library colonies were picked and grown in 300 μl of preculture media in a 96-well deepwell plate. The plate was incubated at 30° C. and shaken at 950 rpm for 22 hours. Next, 50 μl of incubated preculture was removed from each well and mixed into a new 96-well deepwell plate filled with 450 μl of macronutrient medium. The new plate was incubated at 30° C. and shaken at 950 rpm for 20 hours. Finally, 55 μl of feeding media was added into each plate well, and the incubation was continued for another 72 hours.

Metabolite extraction was performed by adding 30 μl of culture to 270 μl of 56% acetonitrile in a new 96-well microtiter plate. The solutions were mixed thoroughly, then centrifuged at 3750 rpm for 10 mins. The soluble layer was removed and diluted with 56% acetonitrile to an appropriate concentration in a 96-well v-bottom microtiter plate. Samples were stored at −20° C. until analysis.

All culturing steps, metabolites extraction, and assays were carried out in 96-well plate format. The media used in this screening protocol is defined below.

Preculture Media. Preculture media is composed of 1.7 g/L YNB without ammonium sulfate and amino acid, 1.92 g/L URA dropout amino acid supplement, 0.375 g/L hemimagnesium L-glutamate, with 1% w/v glucose.

Macronutrient Media. Macronutrient media contains 1.7 g/L YNB without ammonium sulfate and amino acid, 1.92 g/L URA dropout amino acid supplement, 1.5 g/L hemimagnesium L-glutamate, 2.5 g/L yeast extracts, with 2% w/v glucose.

Feeding Media. Feeding media contains 10 g/L KH₂PO₄, 20 g/L MgSO₄ heptahydrate, 19.4 g/L URA dropout amino acid supplement, 17 g/L hemimagnesium L-glutamate, 0.76 g/L uracil, 2% w/v glucose, 38% w/v galactose with 0.1% v/v vitamins supplement, and 1% v/v trace elements. Vitamin and trace elements solutions were prepared according to the protocol of van Hoek et al. (2000).

Quantification Protocol. Same as in Example 1.

Results:

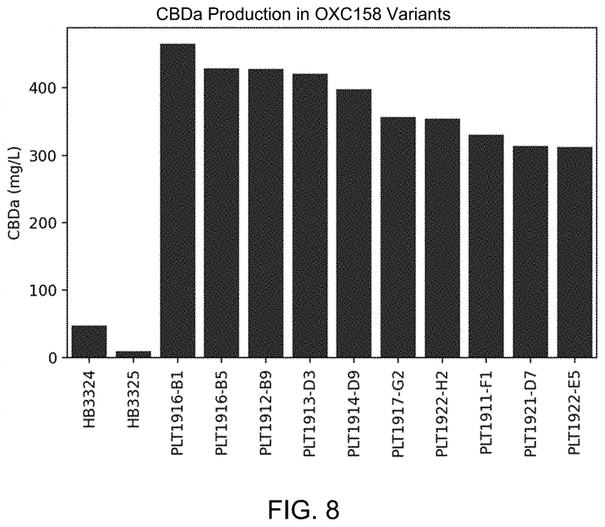

Production of CBDa. Clonal strains harboring a library of mutant OXC158 variants were expressed on plasmids under control of pGAL1 in an CBGa-producing background (HB2652). Comparison with strains expressing OXC158(HB2736) and a non-catalytic control mScarlet (HB2737) facilitated the identification of novel OXC variants with improved activity. FIG. 7 shows cannabinoid production values.

Table 11 shows production of CBDa and upstream metabolites observed in this example.

TABLE 11
Production of CBDa and Upstream Metabolites

| | | | | | | | | Ratio of | Ratio of | Ratio of |
|---|---|---|---|---|---|---|---|---|---|---|
| Sample ID | | | Olivetolic | | | | | CBDa to | CBDa to | CBDa to |
| in the | | Olivetol | Acid | CBGa | THCa | CBDa | | (THCa + | (CBGa + | upstream |
| figure | Enzyme | (mg/L) | (mg/L) | (mg/L) | (mg/L) | (mg/L) | OD600 | CBDa) | CBDa) | metabolites |
| HB2736 | OXC158 | 48.649 | 0.375 | 57.422 | 11.369 | 77.456 | 11.977 | 0.877 | 0.599 | 0.546 |
| HB2737 | mScarlet | 43.204 | 0.902 | 135.105 | 4.143 | 13.033 | 11.894 | 0.769 | 0.112 | |
| PLT1755- | OXC158- | 57.944 | 2.641 | 16.448 | 21.052 | 136.273 | 12.321 | 0.866 | 0.895 | 1.011 |
| H11 | I351G | | | | | | | | | |
| PLT1759- | OXC158- | 55.476 | 2.992 | 24.093 | 19.318 | 125.396 | 12.056 | 0.867 | 0.840 | 0.908 |
| E8 | S367R | | | | | | | | | |
| PLT1745- | OXC158- | 56.839 | 5.923 | 24.792 | 22.330 | 124.016 | 10.625 | 0.847 | 0.835 | 0.847 |
| H2 | Q274G | | | | | | | | | |
| PLT1755- | OXC158- | 56.165 | 5.093 | 17.535 | 20.320 | 122.355 | 11.650 | 0.858 | 0.876 | 0.898 |
| H8 | I351M | | | | | | | | | |
| PLT1762- | OXC158- | 54.780 | 4.918 | 10.877 | 20.917 | 121.047 | 12.542 | 0.853 | 0.918 | 0.958 |
| F6 | V383A | | | | | | | | | |
| PLT1759- | OXC158- | 49.135 | 4.704 | 14.729 | 18.550 | 119.581 | 12.343 | 0.866 | 0.892 | 1.004 |
| G8 | S367Q | | | | | | | | | |
| PLT1759- | OXC158- | 53.155 | 4.555 | 10.917 | 18.757 | 119.130 | 12.659 | 0.864 | 0.917 | 0.968 |
| F10 | S367N | | | | | | | | | |
| PLT1759- | OXC158- | 54.225 | 4.797 | 18.847 | 18.795 | 117.949 | 12.370 | 0.863 | 0.865 | 0.888 |
| G11 | S367R | | | | | | | | | |

### Example 4

Set of CBDa Producing OXC158 Mutants

In this example, an additional set of CBDa producing OXC158 enzyme mutants, derived from combinatorial expression of the single-site mutations identified in Example 3 are prepared.

Materials and Methods:

Genetic Manipulations. Similar methods were used for combinatorial mutant library construction as the Multiple Double-Strand Fragment methods described in Examples 1 and 2. However, some modifications were made to facilitate genomic integration of variant sequences. Mutagenic fragments pertaining to a target mutation were amplified by using corresponding mutagenic primers that were designed to overlap with adjacent fragments. A second overlap-extension PCR was applied to assemble multiple mutagenic fragments in one pot. In addition, the target variant sequences were fused with 3′ and 5′ flanking sequences via an additional overlap-extension PCR to create variant cassettes. Cassettes were then used for integration into the yeast genome via CRISPR-Cas9 techniques (Reider et al., 2017). All DNA was transformed into background strains using the transformation protocol of Geitz & Woods (2006). Modifications made to base strains for Examples 2-5 are outlined in Table 14, below, and point substitutions described in Examples 2-4, wherein amino acid position numbers refer to the OXC154 sequence, are provided in Table 15 below.

Strain growth and Media. Same as in Example 3, with exception to the assay culture incubation time; to facilitate selection of earlier maturing OXas with improved activity, following addition of feeding media, incubation time before extraction was shortened to 48 hours.

Quantification Protocol. Same as in Example 1.

Results:

Production of Cannabidiolic Acid. Clonal strains harboring a library of OXC158 combinatorial mutant variants were expressed from pGAL1 following CRISPR-Cas9 mediated genomic integration in a CBGa-producing background (HB3423). Comparison with strains expressing OXC158 (HB3324) and a non-catalytic control, mScarlet (HB3325), facilitated the identification of novel OXC variants with improved activity.

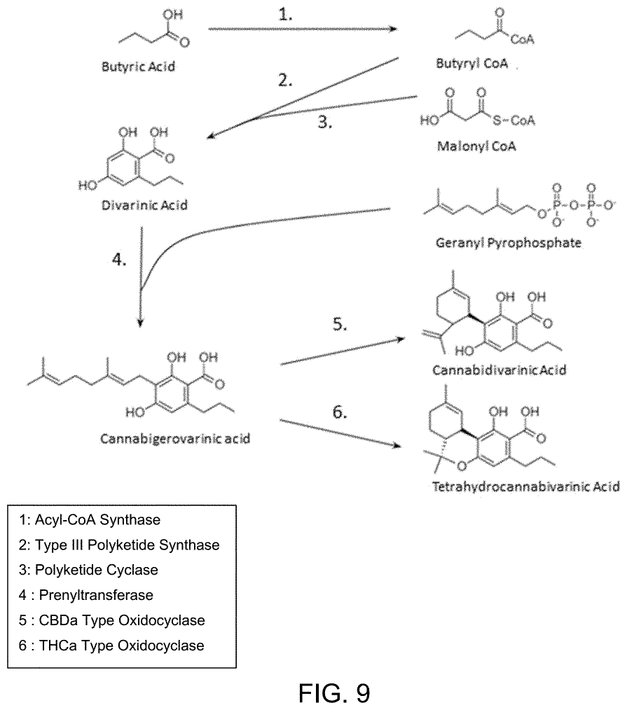

FIG. 8 shows CDa production in strains expressing OXC158 variants identified through a combinatorial library.

Table 12 illustrates production of CDa and upstream metabolites observed in this example.

TABLE 12
Production of CBDa and Upstream Metabolites

| | | | | | | | | Ratio of | Ratio of | Ratio of |
|---|---|---|---|---|---|---|---|---|---|---|
| Sample ID | | | Olivetolic | | | | | CBDa to | CBDa to | CBDa to |
| in the | | Olivetol | Acid | CBGa | THCa | CBDa | | (THCa + | (CBGa + | upstream |
| figure | Enzyme | (mg/L) | (mg/L) | (mg/L) | (mg/L) | (mg/L) | OD600 | CBDa) | CBDa) | metabolites |
| HB3324 | OXC158 | 110.697 | 47.996 | 292.600 | 0.769 | 77.948 | 12.090 | 0.991 | 0.222 | 0.133 |
| HB3325 | mScarlet | 129.350 | 59.252 | 557.170 | 7.443 | 12.545 | 12.072 | 0.641 | 0.023 | 0.014 |
| 1916-B1 | OXC158- | 243.692 | 207.932 | 72.178 | 27.650 | 465.483 | 10.377 | 0.944 | 0.868 | 0.524 |
| | L31E/V383G | | | | | | | | | |
| 1916-B5 | OXC158- | 260.055 | 213.678 | 237.003 | 22.748 | 428.807 | 10.175 | 0.950 | 0.646 | 0.392 |
| | N138T/V383M/ | | | | | | | | | |
| | H515E | | | | | | | | | |
| 1912-B9 | OXC158- | 212.695 | 134.557 | 41.805 | 27.518 | 428.278 | 11.255 | 0.940 | 0.911 | 0.634 |
| | S367K/V383A/ | | | | | | | | | |
| | P513V | | | | | | | | | |
| 1913-D3 | OXC158-V383A | 230.635 | 195.887 | 39.027 | 27.087 | 421.032 | 10.466 | 0.940 | 0.915 | 0.518 |
| 1914-D9 | OXC158-W3A/ | 219.145 | 113.923 | 187.890 | 21.275 | 397.760 | 11.575 | 0.949 | 0.684 | 0.493 |
| | L31E/K226M/ | | | | | | | | | |
| | S367Q/V383M/ | | | | | | | | | |
| | S399G/P513V | | | | | | | | | |
| 1917-G2 | OXC158-W3A/ | 127.282 | 52.465 | 16.403 | 17.580 | 356.487 | 12.007 | 0.953 | 0.956 | 1.022 |
| | N5Q/I351G | | | | | | | | | |
| 1922-H2 | OXC158- | 127.065 | 55.217 | 46.430 | 15.100 | 354.057 | 12.067 | 0.959 | 0.884 | 0.918 |
| | I351G/V383A | | | | | | | | | |
| 1911-F1 | OXC158-L31E/ | 182.950 | 105.485 | 66.557 | 21.540 | 330.195 | 11.671 | 0.939 | 0.836 | 0.550 |
| | Q274G/S367K/ | | | | | | | | | |
| | V383A | | | | | | | | | |
| 1921-D7 | OXC158- | 110.537 | 38.502 | 35.362 | 14.725 | 313.932 | 12.249 | 0.955 | 0.900 | 1.006 |
| | W3A/I351G/ | | | | | | | | | |
| | V383A | | | | | | | | | |
| 1922-E5 | OXC158- | 114.370 | 51.158 | 32.588 | 14.737 | 312.425 | 12.489 | 0.955 | 0.906 | 0.917 |
| | W3A/N5Q/N28E/ | | | | | | | | | |
| | I351G/S367R/ | | | | | | | | | |
| | V383A | | | | | | | | | |

### Example 5

OXC Variants for the Production of CBDVa

Wild type cannabidiolic acid synthase (CBDa synthase or “OXC52” herein), when modified with the insertion of a serine between positions 224 and 225 in the OXC52 sequence, results in OXC154. OXC variants for the production of CBDVa are described herein.

### INTRODUCTION

Cannabidiolic acid synthase (CBDAS) predominantly utilizes cannabigerolic acid (CBGa) as substrate to form CBDa but it can also accept cannabigerovarinic acid (CBGVa) as a precursor to generate cannabidivarinic acid (CBDVa). CBDVa is thought to have a number of useful therapeutic applications such as the treatment of epilepsy and autism (Zamberletti et al., 2021).

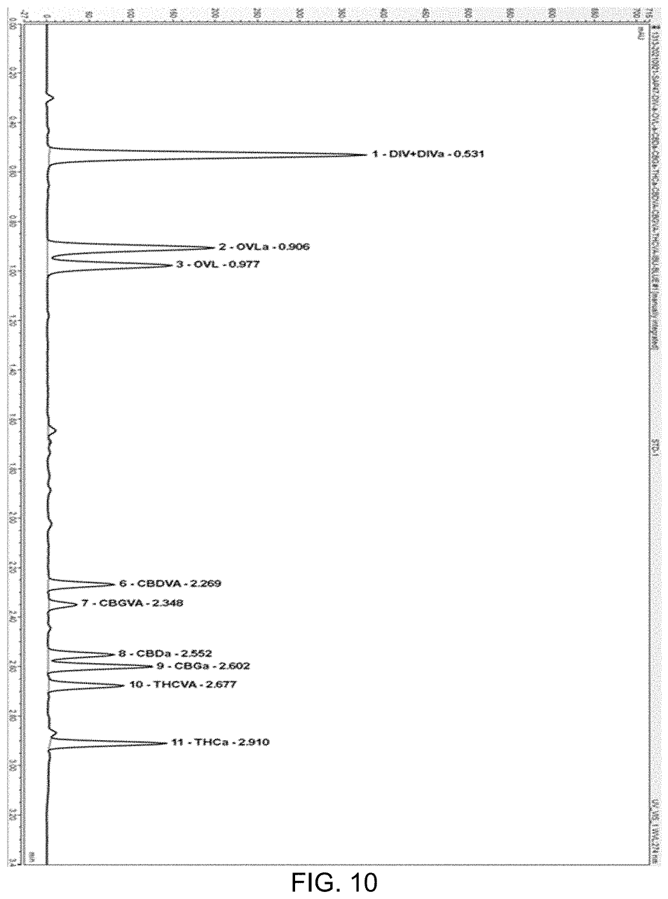

FIG. 9 shows the cannabivarinic acid biosynthesis pathway in Cannabis sativa. CBDVa can be produced in a heterologous host by expressing an appropriate acyl-CoA synthetase, polyketide cyclase, polyketide synthase, prenyltransferase and oxidocyclase in the presence of butyric acid. Butyric acid may be supplied exogenously or produced directly in the host. The oxidocylases described in Examples 1-4 can be used to produce CBDVa in addition to CBDa

Materials and Methods:

Genetic Manipulations. HB42 was used as a base strain to develop all other strains in this experiment. CRISPR and DNA transformation protocols were done as described in Example 4. CBDVa producing strains were generated by genomic integration of type III PKS (PKS73, DNA SEQ ID NO:202), an acyl-activating enzyme (CsAAE1, DNA SEQ ID NO:201), a prenyltransferase (PT254-R2S, SEQ ID NO:155) and an oxidocyclase (OXC52 (AA SEQ ID NO:140), OXC154-S88A/L451G (AA SEQ ID NO:72) or OXC157 which is also referred to herein as: OXC154-R3G/A18E/S60T/G351I/A383V/L451G (AA SEQ ID NO:161; DNA SEQ ID NO:205 or 157) into an appropriate yeast background.

Strain growth and Media. Same as in Example 3, with exception to the assay culture feeding media and incubation time. Following macronutrient growth, feeding media supplemented with 5 mM butyric acid was added to each well and the culture was incubated at 30° C. and shaken at 950 rpm for 96 hours.

Quantification Protocol. The quantification of metabolites was performed using a Thermo Scientific Vanquish™ UHPLC-UV system. The chromatography and UV conditions are described below. Divarin (DIV) and divarinic acid (DIVa, the precursor to varinoid biosynthesis) were not separated on the UV chromatograms and are therefore considered as a single peak.

LC Conditions:

Column: Raptor Biphenyl 100×2.1 mm, 1.8 μm particle size (PN: 9309212)

Guard column: UltraShield UHPLC PreColumn Filter (PN: 24997)

Column temperature: 55° C.

Flow rate: 0.800 mL/min

Eluent A: Water+0.1% Formic Acid

Eluent B: ACN+0.1% Formic Acid

Gradient:

| Time (min) | % B | Flow rate (mL/min) |
|---|---|---|
| 0.00 | 35 | 0.800 |
| 0.30 | 35 | 0.800 |
| 2.30 | 70 | 0.800 |
| 2.30 | 98 | 0.800 |
| 2.50 | 98 | 0.800 |
| 2.50 | 35 | 0.800 |
| 3.40 | 35 | 0.800 |

UV Conditions:

Wavelength: 274 nm

Data collection rate: 4.0 Hz

Response time: 1.00 s

Peak width: 0.100 min

Detection parameters:

| Compound | Retention time (min) |
|---|---|
| DIV + DIVa | 0.530 |
| OVLa | 0.905 |
| OVL | 0.977 |
| CBDa | 2.552 |
| CBGa | 2.602 |
| THCa | 2.910 |
| CBDVa | 2.269 |
| CBGVa | 2.348 |
| THCVa | 2.677 |

Results:

Production of CBDVa

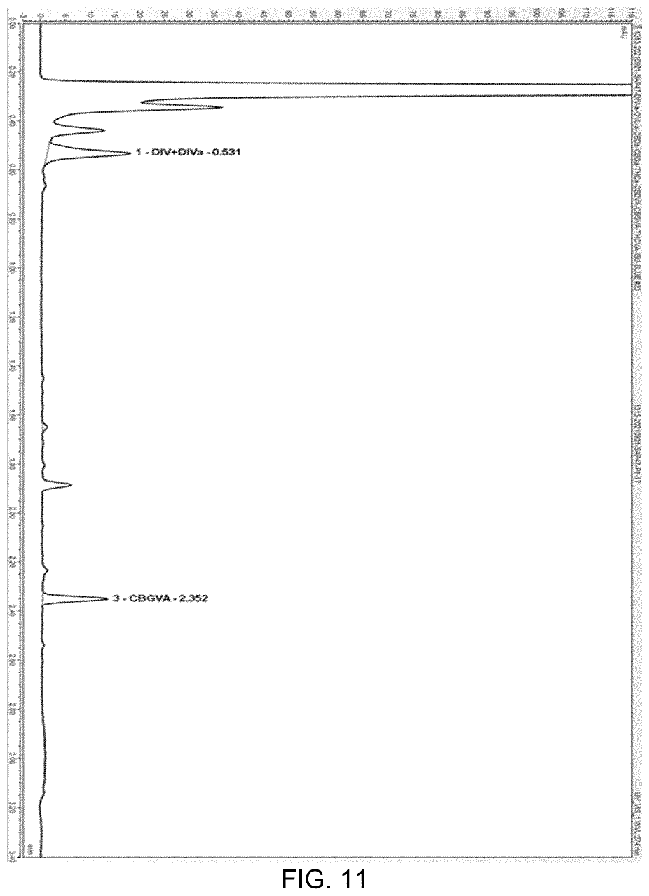

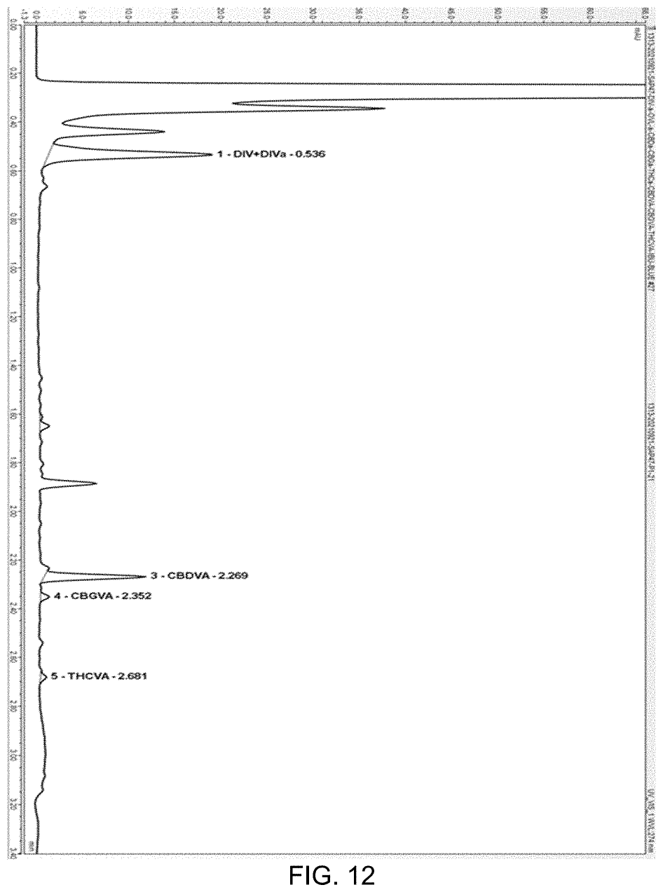

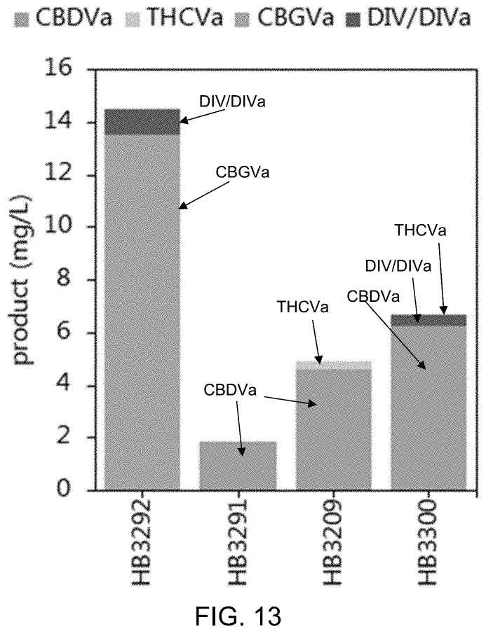

FIG. 10 shows the UV spectra of varinoid standards. FIG. 11 shows UV spectra for CBGVa control strain (HB3292, no oxidocyclase). FIG. 12 shows UV spectra CBDVa strain (HB3291). The presence of a peak at 2.269 minutes in the CBDVa strain (see FIG. 12), but not the CBGVa control (see FIG. 11) indicates the presence of CBDVa.

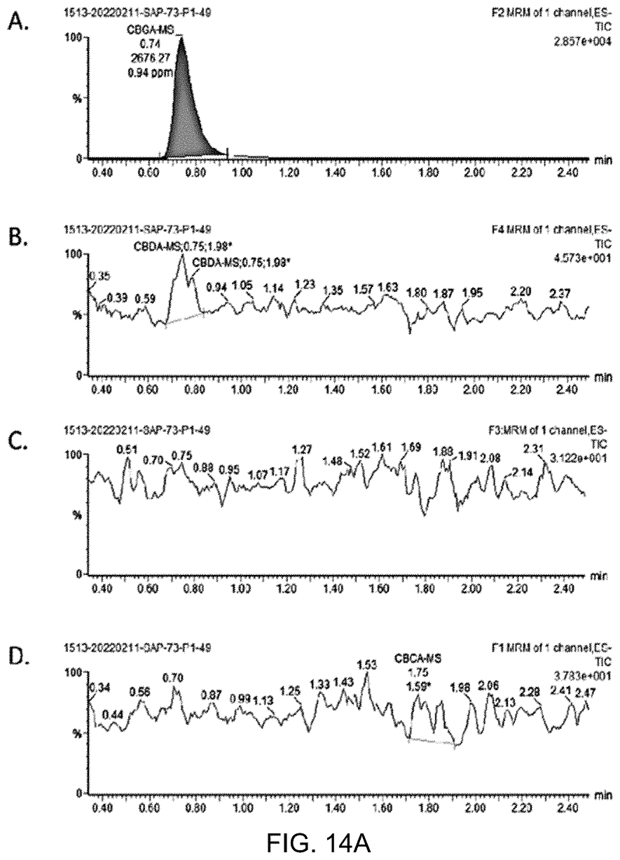

FIG. 13 shows CBDVa and intermediate products THCVa, CBGVa, DIV/DIVa in strains expressing OXC154 variants identified through a combinatorial library.

Table 13 shows CBDVa and intermediate products in strains expressing OXC154 variants identified through a combinatorial library.

TABLE 13
CBDVa and Intermediate Products in
Strains Expressing OXC154 Variants

| | Divarin/Divarinc acid | CBGVa | THCVa | CBDVa |
|---|---|---|---|---|
| strain ID | product (mg/L) | | | |
| HB3292 | 0.95 | 13.55 | 0 | 0 |
| HB3291 | 0 | 0 | 0 | 1.86 |
| HB3209 | 0 | 0 | 0.31 | 4.60 |
| HB3300 | 0.43 | 0 | 0 | 6.26 |

This example illustrates strains so modified are able to produce CBDVa and intermediate products in host cells transformed with a modified CBDa synthase protein according to the described method.

Table 14 shows modifications made to base strains in detail for Examples 2-5.

TABLE 14
List of strains described in Examples 2-5

| Strain # | Plasmid | Genotype/base strain | Notes | Example |
|---|---|---|---|---|
| HB2191 | none | A CBGa-producing Saccharomyces | Base strain for | 2 |
| | | cerevisiae strain, similar to HB965 in | OXC154 clib R2 | |
| | | example 1 | (SEQ ID NO: 141) | |
| HB2522 | PLAS-618 | Saccharomyces cerevisiae base strain | OXC161 (OXC154- | 2 |
| | | HB2191 | G351I/A383V/L451G) | |
| | | | (SEQ ID NO: 127) | |
| HB2523 | PLAS-416 | Saccharomyces cerevisiae base strain | mScarlet | 2 |
| | | HB2191 | | |
| PLT1676-B8 | PLAS-679 | Saccharomyces cerevisiae base strain | OXC154-R3G/A18E/ | 2 |
| | | HB2191 | S60T/G351I/A383V/L451G | |
| | | | (SEQ ID NO: 157) | |
| PLT1675-C8 | PLAS-680 | Saccharomyces cerevisiae base strain | OXC158 (OXC154- | 2 |
| | | HB2191 | R3W/A18E/T49R/V97E/G351I/ | |
| | | | A383V/L451G) | |
| | | | (DNA: SEQ ID NO: 159) | |
| PLT1675-D3 | PLAS-681 | Saccharomyces cerevisiae base strain | OXC154- | 2 |
| | | HB2191 | R3W/A18E/T49R/V97E/G351I/ | |
| | | | A383V/L451G | |
| | | | (DNA: SEQ ID NO: 159) | |
| PLT1675-F10 | PLAS-682 | Saccharomyces cerevisiae base strain | OXC154-R3T/S60T/ | 2 |
| | | HB2191 | G3511/A383V/L451G | |
| | | | (SEQ ID NO: 160) | |
| HB2652 | none | A CBGa-producing Saccharomyces | Base strain for | 3 |
| | | cerevisiae strain, similar to HB965 in | OXC158 SSM | |
| | | example 1 | | |
| HB2736 | PLAS-680 | Saccharomyces cerevisiae base strain | OXC158 | 3 |
| | | HB2652 | | |
| HB2737 | PLAS-416 | Saccharomyces cerevisiae base strain | mScarlet | 3 |
| | | HB2652 | | |
| PLT1755-H11 | PLAS-683 | Saccharomyces cerevisiae base strain | OXC158-I351G | 3 |
| | | HB2652 | (DNA: SEQ ID NO: 165) | |
| PLT1759-E8 | PLAS-684 | Saccharomyces cerevisiae base strain | OXC158-S367R | 3 |
| | | HB2652 | (DNA: SEQ ID NO: | |
| | | | 165 or SEQ ID NO: 166) | |
| PLT1745-H2 | PLAS-685 | Saccharomyces cerevisiae base strain | OXC158-Q274G | 3 |
| | | HB2652 | (DNA: SEQ ID NO: 167) | |
| PLT1755-H8 | PLAS-686 | Saccharomyces cerevisiae base strain | OXC158-I351M | 3 |
| | | HB2652 | (DNA: SEQ ID NO: 168) | |
| PLT1762-F6 | PLAS-687 | Saccharomyces cerevisiae base strain | OXC158-V383A | 3 |
| | | HB2652 | (DNA: SEQ ID NO: 169) | |
| PLT1759-G8 | PLAS-688 | Saccharomyces cerevisiae base strain | OXC158-S367Q | 3 |
| | | HB2652 | (DNA: SEQ ID NO: 170) | |
| PLT1759-F10 | PLAS-689 | Saccharomyces cerevisiae base strain | OXC158-S367N | 3 |
| | | HB2652 | (DNA: SEQ ID NO: 171) | |
| PLT1759-G11 | PLAS-690 | Saccharomyces cerevisiae base strain | OXC158-S367R | 3 |
| | | HB2652 | (DNA: SEQ ID NO: 172) | |
| HB3192 | none | A CBGa-producing Saccharomyces | Base strain for OXC158 | 4 |
| | | cerevisiae strain, similar to HB965 in | combinatorial library | |
| | | example 1 | | |
| HB3324 | none | Saccharomyces cerevisiae base strain | Integrated OXC158 | 4 |
| | | HB3192; pSmGAL2: OXC158 | control for OXC158 | |
| | | | combinatorial library | |
| HB3325 | PLAS-525 | Saccharomyces cerevisiae base strain | Cas9 plasmid control | 4 |
| | | HB3192 | | |
| PLT1916-B1 | none | Saccharomyces cerevisiae base strain | OXC158-L31E/V383G | 4 |
| | | HB3192; pSmGAL2: OXC158- | (DNA SEQ ID NO: 181) | |
| | | L31E/V383G | | |
| PLT1916-B5 | none | Saccharomyces cerevisiae base strain | OXC158-N138T/V383M/H515E | 4 |
| | | HB3192; pSmGAL2: OXC158- | (DNA SEQ ID NO: 182) | |
| | | N138T/V383M/H515E | | |
| PLT1912-B9 | none | Saccharomyces cerevisiae base strain | OXC158-S367K/V383A/P513V | 4 |
| | | HB3192; pSmGAL2: OXC158- | (DNA SEQ ID NO: 183) | |
| | | S367K/V383A/P513V | | |
| PLT1913-D3 | none | Saccharomyces cerevisiae base strain | OXC158-V383A | 4 |
| | | HB3192; pSmGAL2: OXC158-V383A | (DNA SEQ ID NO: 184) | |
| PLT1914-D9 | none | Saccharomyces cerevisiae base strain | OXC158- | 4 |
| | | HB3192; pSmGAL2: OXC158- | W3A/L31E/K226M/S367Q/ | |
| | | W3A/L31E/K226M/S367Q/V383M/S399G/ | V383M/S399G/P513V | |
| | | P513V | (DNA SEQ ID NO: 185) | |
| PLT1917-G2 | none | Saccharomyces cerevisiae base strain | OXC158-W3A/N5Q/I351G | 4 |
| | | HB3192; pSmGAL2: OXC158 - | | |
| | | W3A/N5Q/I351G | | |
| PLT1922-H2 | none | Saccharomyces cerevisiae base strain | OXC158-I351GA/383A | 4 |
| | | HB3192; pSmGAL2: OXC158 - | (SEQ ID NO: 186) | |
| | | I351G/V383A | | |
| PLT1911-F1 | none | Saccharomyces cerevisiae base strain | OXC158- | 4 |
| | | HB3192; pSmGAL2: OXC158- | L31E/Q274G/S367K/V383A | |
| | | L31E/Q274G/S367K/V383A | | |
| PLT1921-D7 | none | Saccharomyces cerevisiae base strain | OXC158-W3A/I351G/V383A | 4 |
| | | HB3192; pSmGAL2: OXC158 - | (DNA SEQ ID NO: 187) | |
| | | W3A/I351G/V383A | | |
| PLT1922-E5 | none | Saccharomyces cerevisiae base strain | OXC158- | 4 |
| | | HB3192; pSmGAL2: OXC158 - | W3A/N5Q/N28E/I351G/ | |
| | | W3A/N5Q/N28E/I351G/S367R/V383A | S367R/V383A | |
| | | | (DNA SEQ ID NO: 188) | |
| HB863 | None | CEN.PK2; ΔLEU2; ΔURA3; | | 5 |
| HB3292 | none | Saccharomyces cerevisiae | Base strain for CBGVa | 5 |
| | | CEN.PK2; ΔLEU2; ΔURA3; | production | |
| | | ERG20-K197E | | |
| | | ALD6; ACS1-L641P; NpgA; MAF1; | | |
| | | ΔpACC1::pPGK1: ACC1; tHMGR1; IDI1; | | |
| | | pTEF1: CsOAC; pGAL1: CsAAE1; | | |
| | | pGAL1: PT254-R2S; | | |
| | | pGAL1: PKS73 | | |
| HB3291 | none | Saccharomyces cerevisiae base strain | Cannabis sativa CBDAS | 5 |
| | | HB3292 | integrated control for CBDVa | |
| | | pTEF1: OXC52 | production | |
| HB3209 | none | Saccharomyces cerevisiae base strain | Integrated OXC154- | 5 |
| | | HB3292 | S88A/L451G | |
| | | pTEF1: OXC154-S88A/L451G | (DNA SEQ ID NO: 4) | |
| HB3300 | none | Saccharomyces cerevisiae base strain | Integrated OXC154- | 5 |
| | | HB3292 | R3G/A18E/S60T/G351I/ | |
| | | pGAL1: OXC154- | A383V/L451G | |
| | | R3G/A18E/S60T/G351I/A383V/L451G | | |

Table 15 lists point substitutions described in Examples 2-4. Amino acid position numbers refer to the OXC154 sequence. Table 8, above, lists other substitutions mentioned herein.

TABLE 15
Point Substitutions Described in Examples 2-4
Amino acid position numbers refer to the OXC154 or OX158 sequence

| | | Number of occurrences in |
|---|---|---|
| | | SEQ ID NOs: 161-164; |
| Substitution | Type | 173-180; 189-196 |
| W3A | Non-conservative | 4 |
| N5Q | Conservative | 2 |
| N28E | Non-conservative | 1 |
| L31E | Non-conservative | 3 |
| Q274G | Non-conservative | 2 |
| I351G | Conservative | 5 |
| I351M | Conservative | 1 |
| S367Q | Conservative | 2 |
| S367N | Conservative | 1 |
| S367R | Non-conservative | 3 |
| S367K | Non-conservative | 2 |
| V383A | Conservative | 7 |
| V383M | Conservative | 2 |
| V383G | Conservative | 1 |
| S399G | Non-conservative | 1 |
| P513V | Non-conservative | 2 |
| H515E | Non-conservative | 1 |

Table 16 shows plasmids used herein.

TABLE 16
Plasmids

| # | Plasmid Name | SEQ ID NO. | Description | Selection |
|---|---|---|---|---|
| 72 | PLAS-679 | SEQ ID | OXC154-R3G/A18E/S60T/G351I/ | Uracil |
| | | NO. 157 | A383V/L451G-VB40 | |
| 73 | PLAS-680 | SEQ ID | OXC154-R3W/A18E/T49R/V97E/ | Uracil |
| | | NO. 158 | G351I/A383V/L451G(OXC158)-VB40 | |
| 74 | PLAS-681 | SEQ ID | PLT1675-D3: OXC154- | Uracil |
| | | NO. 159 | R3W/A18E/T49R/V97E/G351I/ | |
| | | | A383V/L451G-VB40 | |
| 75 | PLAS-682 | SEQ ID | PLT1675-F10: OXC154-R3T/ | Uracil |
| | | NO. 160 | S60T/G351I/A383V/L451G-VB40 | |
| 76 | PLAS-683 | SEQ ID | PLT1755-H11: OXC158-I351G | Uracil |
| | | NO. 165 | | |
| 77 | PLAS-684 | SEQ ID | PLT1759-E8: OXC158-S367R | Uracil |
| | | NO. 166 | | |
| 78 | PLAS-685 | SEQ ID | PLT1745-H2: OXC158-Q274G | Uracil |
| | | NO. 167 | | |
| 79 | PLAS-686 | SEQ ID | PLT1755-H8: OXC158-I351M | Uracil |
| | | NO. 168 | | |
| 80 | PLAS-687 | SEQ ID | PLT1762-F6: OXC158-V383A | Uracil |
| | | NO. 169 | | |
| 81 | PLAS-688 | SEQ ID | PLT1759-G8: OXC158-S367Q | Uracil |
| | | NO. 170 | | |
| 82 | PLAS-689 | SEQ ID | PLT1759-F10: OXC158-S367N | Uracil |
| | | NO. 171 | | |
| 83 | PLAS-690 | SEQ ID | PLT1759-G11: OXC158-S367R | Uracil |
| | | NO. 172 | | |
| 84 | PLAS-635 | SEQ ID | pCAS-GRN248 | G418 |
| | | NO. 197 | | |

Table 17 shows further sequences described herein. Assigned descriptive names for sequences indicate the starting sequence from which mutations are made, which may be for example “OXC154” or “OXC158”. Where OXC154 is indicated, the listed mutated residues in the descriptive name are changed from SEQ ID NO:141. Where OXC158 is indicated in the descriptive name, the listed mutations in the descriptive indicate a change from those residues indicated in the protein of SEQ ID NO:162. For example, SEQ ID NO:195 (Protein), indicated as DNA SEQ ID NO:187, is assigned “OXC158-W3A/I351G/V383A” within its descriptive name. Thus, for this sequence the mutations from SEQ ID NO: 141 are firstly those of OXC158 (as in SEQ ID NO:162, specifically: R3W/A18E/T49R/V97E/G351I/A383V/L451 G), and from these mutations, further mutations are indicated as W3A/I351 G/V383A. Notably, this means that in SEQ ID NO:195, residue 3 is A, residue 351 is G, and residue 383 is A whereas residue 18 is E, residue 49 is R, residue 97 is E, and residue 451 is G.

TABLE 17
Sequences

| | | | Length of | Position of coding |
|---|---|---|---|---|
| SEQ ID NO: | Description | DNA/Protein | sequence | sequence |
| SEQ ID | OXC154- | DNA | 7266 | 2925 to 4499 |
| NO. 157 | R3G/A18E/S60T/G351I/ | | | |
| | A383V/L451G-VB40 | | | |
| SEQ ID | OXC154- | DNA | 7266 | 2925 to 4499 |
| NO. 158 | R3W/A18E/T49R/V97E/G351I/A383V/ | | | |
| | L451G(OXC158)-VB40 | | | |
| SEQ ID | PLT1675-D3: OXC154- | DNA | 7266 | 2925 to 4499 |
| NO. 159 | R3W/A18E/T49R/V97E/ | | | |
| | G351I/A383V/L451G-VB40 | | | |
| SEQ ID | PLT1675-F10: OXC154- | DNA | 7266 | 2925 to 4499 |
| NO. 160 | R3T/S60T/G351I/A383V/L451G-VB40 | | | |
| SEQ ID | OXC154- | Protein | 524 | All |
| NO. 161 | R3G/A18E/S60T/G351I/A383V/L451G-VB40 | | | |
| SEQ ID | OXC154- | Protein | 524 | All |
| NO. 162 | R3W/A18E/T49R/V97E/G351I/A383V/ | | | |
| | L451G(OXC158)-VB40 (which may be | | | |
| | alternatively referenced herein as | | | |
| | “OXC158”) | | | |
| SEQ ID | PLT1675-D3: OXC154- | Protein | 524 | All |
| NO. 163 | R3W/A18E/T49R/V97E/ | | | |
| | G351I/A383V/L451G-VB40 | | | |
| SEQ ID | PLT1675-F10: OXC154- | Protein | 524 | All |
| NO. 164 | R3T/S60T/G351I/A383V/L451G-VB40 | | | |
| SEQ ID | PLT1755-H11: OXC158-I351G | DNA | 7266 | 2925 to 4499 |
| NO. 165 | | | | |
| SEQ ID | PLT1759-E8: OXC158-S367R(=CGG) | DNA | 7266 | 2925 to 4499 |
| NO. 166 | | | | |
| SEQ ID | PLT1745-H2: OXC158-Q274G | DNA | 7266 | 2925 to 4499 |
| NO. 167 | | | | |
| SEQ ID | PLT1755-H8: OXC158-I351M | DNA | 7266 | 2925 to 4499 |
| NO. 168 | | | | |
| SEQ ID | PLT1762-F6: OXC158-V383A | DNA | 7266 | 2925 to 4499 |
| NO. 169 | | | | |
| SEQ ID | PLT1759-G8: OXC158-S367Q | DNA | 7266 | 2925 to 4499 |
| NO. 170 | | | | |
| SEQ ID | PLT1759-F10: OXC158-S367N | DNA | 7266 | 2925 to 4499 |
| NO. 171 | | | | |
| SEQ ID | PLT1759-G11: OXC158-S367R(=AGG) | DNA | 7266 | 2925 to 4499 |
| NO. 172 | | | | |
| SEQ ID | PLT1755-H11: OXC158-I351G | Protein | 524 | All |
| NO. 173 | | | | |
| SEQ ID | PLT1759-E8: OXC158-S367R(=CGG) | Protein | 524 | All |
| NO. 174 | | | | |
| SEQ ID | PLT1745-H2: OXC158-Q274G | Protein | 524 | All |
| NO. 175 | | | | |
| SEQ ID | PLT1755-H8: OXC158-I351M | Protein | 524 | All |
| NO. 176 | | | | |
| SEQ ID | PLT1762-F6: OXC158-V383A | Protein | 524 | All |
| NO. 177 | | | | |
| SEQ ID | PLT1759-G8: OXC158-S367Q | Protein | 524 | All |
| NO. 178 | | | | |
| SEQ ID | PLT1759-F10: OXC158-S367N | Protein | 524 | All |
| NO. 179 | | | | |
| SEQ ID | PLT1759-G11: OXC158-S367R(=AGG) | Protein | 524 | All |
| NO. 180 | | | | |
| SEQ ID | PLT1916-B1: OXC158-L31E/V383G | DNA | 3690 | 1379 to 2932 |
| NO. 181 | | | | |
| SEQ ID | PLT1916-B5: OXC158- | DNA | 3690 | 1379 to 2932 |
| NO. 182 | N138T/V383M/H515E | | | |
| SEQ ID | PLT1912-B9: OXC158- | DNA | 3690 | 1379 to 2932 |
| NO. 183 | S367K/V383A/P513V | | | |
| SEQ ID | PLT1913-D3: OXC158-V383A | DNA | 3690 | 1379 to 2932 |
| NO. 184 | | | | |
| SEQ ID | PLT1914-D9: OXC158- | DNA | 3690 | 1379 to 2932 |
| NO. 185 | W3A/L31E/K226M/S367Q/ | | | |
| | V383M/S399G/P513V | | | |
| SEQ ID | PLT1922-H2: OXC158-I351G/V383A | DNA | 3690 | 1379 to 2932 |
| NO. 186 | | | | |
| SEQ ID | PLT1921-D7: OXC158- | DNA | 3690 | 1379 to 2932 |
| NO. 187 | W3A/I351G/V383A | | | |
| SEQ ID | PLT1922-E5: OXC158- | DNA | 3690 | 1379 to 2932 |
| NO. 188 | W3A/N5Q/N28E/I351G/S367R/V383A | | | |
| SEQ ID | PLT1916-B1: OXC158-L31E/V383G | Protein | 524 | All |
| NO. 189 | | | | |
| SEQ ID | PLT1916-B5: OXC158- | Protein | 524 | All |
| NO. 190 | N138T/V383M/H515E | | | |
| SEQ ID | PLT1912-B9: OXC158- | Protein | 524 | All |
| NO. 191 | S367K/V383A/P513V | | | |
| SEQ ID | PLT1913-D3: OXC158-V383A | Protein | 524 | All |
| NO. 192 | | | | |
| SEQ ID | PLT1914-D9: OXC158- | Protein | 524 | All |
| NO. 193 | W3A/L31E/K226M/S367Q/ | | | |
| | V383M/S399G/P513V | | | |
| SEQ ID | PLT1922-H2: OXC158-I351G/V383A | Protein | 524 | All |
| NO. 194 | | | | |
| SEQ ID | PLT1921-D7: OXC158- | Protein | 524 | All |
| NO. 195 | W3A/I351G/V383A | | | |
| SEQ ID | PLT1922-E5: OXC158- | Protein | 524 | All |
| NO. 196 | W3A/N5Q/N28E/I351G/S367R/V383A | | | |
| SEQ ID | pCAS-GRN248 | DNA | 9706 | gRNA GRN248: |
| NO. 197 | | | | 1986-2105 |
| | | | | CAS9: 5279-9379 |
| SEQ ID | pSmGAL2: OXC158 | DNA | 3690 | 1379 to 2932 |
| NO. 198 | | | | |
| SEQ ID | ΔpACC1::pPGK1: ACC1 | DNA | 8989 | 1288 to 7989 |
| NO. 199 | | | | |
| SEQ ID | pTEF1: CsOAC | DNA | 2709 | 1374 to 1682 |
| NO. 200 | | | | |
| SEQ ID | pGAL1: CsAAE1 | DNA | 4288 | 1289 to 3454 |
| NO. 201 | | | | |
| SEQ ID | pGAL1: PKS73 | DNA | 2516 | 728 to 1825 |
| NO. 202 | | | | |
| SEQ ID | pTEF1: OXC52 | DNA | 3598 | 1112 to 2959 |
| NO. 203 | | | | |
| SEQ ID | pTEF1: OXC154-S88A/L451G | DNA | 3601 | 1112 to 2962 |
| NO. 204 | | | | |
| SEQ ID | pGAL1: OXC154- | DNA | 3390 | 922 to 2751 |
| NO. 205 | R3G/A18E/S60T/G351I/A383V/L451G | | | |
| SEQ ID | 3′ Histidine tag | AA | | |
| NO. 206 | | | | |
| SEQ ID | OXC154 with variant residues | Protein | 524 | All |
| NO. 207 | | | | |

Table 18 shows modifications to base strains used.

TABLE 18
Modifications to Base Strains

| | | | Integration | | |
|---|---|---|---|---|---|
| | Modification | SEQ | Region/ | | Genetic Structure |
| # | name | ID NO. | Plasmid | Description | of Sequence |
| 1 | ALD6; | SEQ | Flagfeldt | Acetaldehyde dehydrogenase | Fgf19Up::pTDH3: ALD6: |
| | ACS1- | ID NO. | Site 19 | (ALD6) | tADH1::pTEF1: SeACS1- |
| | L641P | 148 | integration | from S. cerevisiae and | L641P: tPRM9::Fgf19Down |
| | | | (Flagfeldt et | acetyl-CoA | |
| | | | al., 2009) | synthase from Salmonella enterica | |
| | | | | (SeACS1-L641P). Will | |
| | | | | allow greater | |
| | | | | accumulation of | |
| | | | | acetyl-CoA in the cell. | |
| | | | | (Shiba et al., 2007) | |
| 2 | NpgA | SEQ | Flagfeldt | Phosphopantetheinyl | Fgf14Up::pTEF1: NpgA: |
| | | ID NO. | Site 14 | transferase from | tPRM9::Fgf14Down |
| | | 142 | integration | Aspergillus niger. | |
| | | | (Flagfeldt et | Accessory Protein for | |
| | | | al., 2009) | DiPKS. (Kim et al., 2015) | |
| 3 | MAF1 | SEQ | Flagfeldt | MAF1 is a regulator of | Fgf5Up::pTEF1: Maf1: |
| | | ID NO. | Site 5 | tRNA biosynthesis. | tPRM9::Fgf5Down |
| | | 149 | integration | Overexpression in S. cerevisiae | |
| | | | (Flagfeldt et | has demonstrated higher | |
| | | | al., 2009) | monoterpene (GPP) | |
| | | | | yields. (Liu et | |
| | | | | al., 2013) | |
| 4 | Erg20K197E | SEQ | Chromosomal | Mutant of Erg20 protein | Tpi1t: ERG20K197E: |
| | | ID NO. | modification | that diminishes | Cyc1t::Tef1p: KanMX: Tef1t |
| | | 150 | | FPP synthase activity | |
| | | | | creating greater | |
| | | | | pool of GPP precursor. | |
| | | | | Negatively affects | |
| | | | | growth phenotype.(Oswald | |
| | | | | et al., 2007) | |
| 5 | tHMGR1; | SEQ | USER Site | Overexpression of truncated | USERX- |
| | IDI1 | ID NO. | X-3 | Hmg1 and Idi1 proteins that | 3Up::pTDH3: tHMGR1: |
| | | 152 | integration | have been previously | tADH1::pTEF1: IDI1: |
| | | | (Jensen et | identified to be bottlenecks in the | tPRM9::USERX-3Down |
| | | | al., 2014) | S. cerevisiae terpenoid | |
| | | | | pathway responsible | |
| | | | | for GPP production | |
| | | | | (Ro et al., 2006) | |
| 6 | pGAL1: PT254- | SEQ | Flagfeldt | The Cannabis sativa | Fgf20Up::pGAL1: PT254- |
| | R2S | ID NO. | Site 20 | prenyltransferase | R2S: tCYC1::Fgf20Down |
| | | 155 | integration | PT254 allows CBGa to | |
| | | | (Flagfeldt et | be produced from | |
| | | | al., 2009) | olivetolic acid and | |
| | | | | geranyl pyrophosphate | |
| | | | | (Luo et al., 2019). The | |
| | | | | N-terminal arginine | |
| | | | | of this enzyme has been | |
| | | | | replaced with a | |
| | | | | serine in order to enhance | |
| | | | | protein stability | |
| | | | | in accordance with N-end rule | |
| | | | | (Varshavsky 1996). | |
| 7 | pSmGAL2: OXC158 | SEQ | USER Site | Mutated Cannabis sativa | USERXI- |
| | | ID NO. | XI-1 | oxidocyclase | 1Up::pSmGAL2: OXC158: |
| | | 198 | integration | (CBDAS) protein allows | tCYC1::USERXI-1Down |
| | | | (Jensen et | the production of | |
| | | | al., 2014) | CBDa from CBGa. | |
| 8 | ΔpACC1::pPGK1: ACC1 | SEQ | Chromosomal | Leads to greater | pACC1::pPGK1: ACC1: tACC1 |
| | | ID NO. | Modification | malonyl-CoA pools. The | |
| | | 199 | | promoter of ACC1 was | |
| | | | | changed to a | |
| | | | | constitutive promoter | |
| | | | | for higher | |
| | | | | expression. (Shi et | |
| | | | | al., 2014) | |
| 9 | pTEF1: CsOAC | SEQ | Flagfeldt | The Cannabis sativa | Fgf16Up::pTEF1: CsOAC: |
| | | ID NO. | Site 16 | olivetolic acid | tENO2::Fgf16Down |
| | | 200 | integration | cyclase (CsOAC) protein | |
| | | | (Flagfeldt et | allows the production of | |
| | | | al., 2009) | divarinic acid from a | |
| | | | | polyketide precursor. | |
| 10 | pGAL1: CsAAE1 | SEQ | USER Site | The Cannabis sativa | USERXI- |
| | | ID NO. | XI-2 | acyl-activating | 2Up::GAL1: CsAAE1: |
| | | 201 | integration | enzyme (CsAAE1) | tCYC1::USERXI-2Down |
| | | | (Jensen et | | |
| | | | al., 2014) | | |
| 11 | pGAL1: PKS73 | SEQ | USER Site | Type III PKS domain from | USERX- |
| | | ID NO. | X-4 | PKS from | 4Up::pGAL1: PKS73: |
| | | 202 | integration | P. pallidum. PKS113 | tCYC1::USERX-4Down |
| | | | (Jensen et | | |
| | | | al., 2014) | | |
| 12 | pTEF1: OXC52 | SEQ | Apel-3 | The Cannabis sativa CBDa | Apel- |
| | | ID NO. | Integration | synthase | 3Up::pTEF1: OXC52: |
| | | 203 | Site (Reider | (OXC52) protein allows | tCYC1::Apel-3Down |
| | | | et al., 2017) | for the production | |
| | | | | of CBDVa from CBGVa | |
| 13 | pTEF1: OXC154- | SEQ | Apel-3 | Mutated Cannabis sativa CBDa | Apel-3Up::pTEF1: |
| | S88A/L451G | ID NO. | Integration | synthase (OXC154-S88A/L451G) | OXC154-S88A/L451G: |
| | | 204 | Site (Reider | protein allows for | tCYC1::Apel-3Down |
| | | | et al., 2017) | improved production of | |
| | | | | CBDVa from CBGVa | |
| 14 | pGAL1: OXC154- | SEQ | Apel-3 | Mutated Cannabis sativa | Apel-3Up::pGAL1: (XC154- |
| | R3G/A18E/S60T/ | ID NO. | Integration | CBDa synthase (OXC154-R3G/A18E/ | R3G/A18E/S60T/G351I/ |
| | G351I/A383V/ | 205 | Site (Reider | S60T/G351I/A383V/L451G) | A383V/L451G: |
| | L451G | | et al., 2017) | protein allows for improved | tCYC1::Apel-3Down |
| | | | | production of CBDVa | |
| | | | | from CBGVa | |
| 15 | PLT1916-B1: OXC158- | SEQ | USER Site | Mutated Cannabis sativa | USERXI-1Up::pSmGAL2: |
| | L31E/V383G | ID NO. | XI-1 | oxidocyclase (CBDAS) protein | OXC158-L31E/V383G: |
| | | 181 | integration | allows the production of | tCYC1::USERXI-1Down |
| | | | (Jensen et | CBDa from CBGa. | |
| | | | al., 2014) | | |
| 16 | PLT1916-B5: OXC158- | SEQ | USER Site | Mutated Cannabis sativa | USERXI-1Up::pSmGAL2: |
| | N138T/V383M/ | ID NO. | XI-1 | oxidocyclase (CBDAS) protein | OXC158-N138T/V383M/H515E: |
| | H515E | 182 | integration | allows the production of | tCYC1::USERXI-1Down |
| | | | (Jensen et | CBDa from CBGa. | |
| | | | al., 2014) | | |
| 17 | PLT1912-B9: OXC158- | SEQ | USER Site | Mutated Cannabis sativa | USERXI- |
| | S367K/V383A/P513V | ID NO. | XI-1 | oxidocyclase (CBDAS) protein | 1Up::pSmGAL2: OXC158- |
| | | 183 | integration | allows the production of | S367K/V383A/P513V: |
| | | | (Jensen et | CBDa from CBGa. | tCYC1::USERXI-1Down |
| | | | al., 2014) | | |
| 18 | PLT1913-D3: OXC158- | SEQ | USER Site | Mutated Cannabis sativa | USERXI- |
| | V383A | ID NO. | XI-1 | oxidocyclase (CBDAS) protein | 1Up::pSmGAL2: OXC158- |
| | | 184 | integration | allows the production of | V383A: tCYC1::USERXI-1Down |
| | | | (Jensen et | CBDa from CBGa. | |
| | | | al., 2014) | | |
| 19 | PLT1914-D9: OXC158- | SEQ | USER Site | Mutated Cannabis sativa | USERXI- |
| | W3A/L31E/K226M/ | ID NO. | XI-1 | oxidocyclase (CBDAS) protein | 1Up::pSmGAL2: OXC158- |
| | S367Q/V383M/S399G/ | 185 | integration | allows the production of | W3A/L31E/K226M/S367Q/V383M/S399G/ |
| | P513V | | (Jensen et | CBDa from CBGa. | P513V: tCYC1::USERXI-1Down |
| | | | al., 2014) | | |
| 20 | PLT1922-H2: OXC158- | SEQ | USER Site | Mutated Cannabis sativa | USERXI- |
| | I351G/ | ID NO. | XI-1 | oxidocyclase (CBDAS) protein | 1Up::pSmGAL2: OXC158- |
| | V383A | 186 | integration | allows the production of | I351G/V383A: |
| | | | (Jensen et | CBDa from CBGa. | tCYC1::USERXI-1Down |
| | | | al., 2014) | | |
| 21 | PLT1921-D7: OXC158-W3A/ | SEQ | USER Site | Mutated Cannabis sativa | USERXI- |
| | I351G/ | ID NO. | XI-1 | oxidocyclase (CBDAS) protein | 1Up::pSmGAL2: OXC158- |
| | V383A | 187 | integration | allows the production of | W3A/I351G/V383A: |
| | | | (Jensen et | CBDa from CBGa. | tCYC1::USERXI-1Down |
| | | | al., 2014) | | |
| 22 | PLT1922-E5: OXC158- | SEQ | USER Site | Mutated Cannabis sativa | USERXI- |
| | W3A/N5Q/ | ID NO. | XI-1 | oxidocyclase (CBDAS) protein | 1Up::pSmGAL2: OXC158- |
| | N28E/I351G/ | 188 | integration | allows the production of | W3A/N5Q/N28E/I351G/S367R/ |
| | S367R/ | | (Jensen et | CBDa from CBGa. | V383A: tCYC1::USERXI-1Down |
| | V383A | | al., 2014) | | |

### Example 6

OXC Variants for the Production of CBCa

CBCa is a naturally occurring phytocannabinoid similar in structure to THCa and CBDa. CBCa is produced when CBGa is brought into contact with an appropriate oxidocyclase (OXC). OXC variants for the production of CBCa are described herein.

Materials and Methods:

Genetic Manipulations:

HB42 was used as a base strain to develop all other strains in this experiment. CRISPR and DNA transformation protocols were done as described in Example 4.

Strain Growth and Media:

Strains were grown in a production media with a composition of 1.7 g/L YNB without ammonium sulfate and amino acid, 1.92 g/L URA dropout amino acid supplement, 1.5 g/L hemi-magnesium L-glutamate, 2.5 g/L yeast extracts, 1 g/L monopotassium phosphate, 2 g/L magnesium sulfate heptahydrate, with 2% w/v glucose and 3.8% w/v galactose (Sigma-Aldrich Canada). The culture was incubated at 30° C. for four days (96 hours).

Experimental Conditions:

Each variant was tested in three replicates and each replicate was clonally derived from single colonies. All strains were grown in 500 μL of media for 96 hours in 96-well deepwell plates. The 96-well deepwell plates were incubated at 30° C. and shaken at 950 rpm for 96 hrs.

Metabolite extraction was performed by adding 900 μl of an 83% Acetonitrile solution to 100 μl of culture in a new 96-well deepwell plate, followed by resuspension 10 times with a 200 ul pipette. The solutions were then centrifuged at 3750 rpm for 5 min. 200 μl of the soluble layer was removed and stored in a 96-well v-bottom microtiter plate. Samples were stored at −20° C. until analysis.

Metabolite extraction was conducted by thoroughly mixing 30 μL of sample culture with 270 μL of 56% acetonitrile in a 96-well microtiter plate, then centrifuged at 3750 rpm for 10 mins. The soluble layer was removed and diluted with 56% acetonitrile to an appropriate concentration in a new 96-well microtiter plate and stored at −20° C. until analysis.

Samples were quantified using HPLC-MS analysis.

Quantification Protocol:

The quantification of CBGa, THCa, CBDa and CBCa was performed using HPLC-MS on a Acquity UPLC-TQD MS. The chromatography and MS conditions are described below.

LC Conditions

Column: ACQUITY UPLC 50×1 mm, 1.8 μm particle size; Column temperature: 45° C.; Flow rate: 0.20 ml/min; Eluent A: Water 0.1% formic acid; Eluent B: Acetonitrile 0.1% formic acid; Gradient is shown in Table 19.

TABLE 19
Gradient

| Time (min) | % B |
|---|---|
| 0 | 75 |
| 2.5 | 75 |

ESI-MS Conditions

The following conditions were utilized: Capillary: 2.7 (kV); Source temperature: 150° C.; Desolvation gas temperature: 250° C.; Desolvation gas flow (nitrogen): 500 L/hour; Cone gas flow (nitrogen): 50 L/hour. Detection parameters are shown in Table 20.

TABLE 20
Detection Parameters

| | CBGa | THCa | CBDa | CBCa |
|---|---|---|---|---|
| Retention time (min) | 0.75 | 1.54 | 0.74 | 1.75 |
| Transition (m/z) | 359.2→341.2 | 357.2→313.2 | 357.2→245.1 | 357.1 → 191.1 |
| Mode | ES−, MRM | ES−, MRM | ES−, MRM | ES−, MRM |
| Cone | 30 | 45 | 45 | 44 |
| Cone (V) | 20 | 20 | 20 | 20 |

Results:

Production of CBCa in S. cerevisiae using oxidocyclases was observed.

CBDa, THCa and CBCa were producing by transforming a CBGa producing strain (HB3167) with plasmids containing OXC52 (SEQ ID NO:1), OXC157 (SEQ ID NO:205) and OXC158 (SEQ ID NO:158).

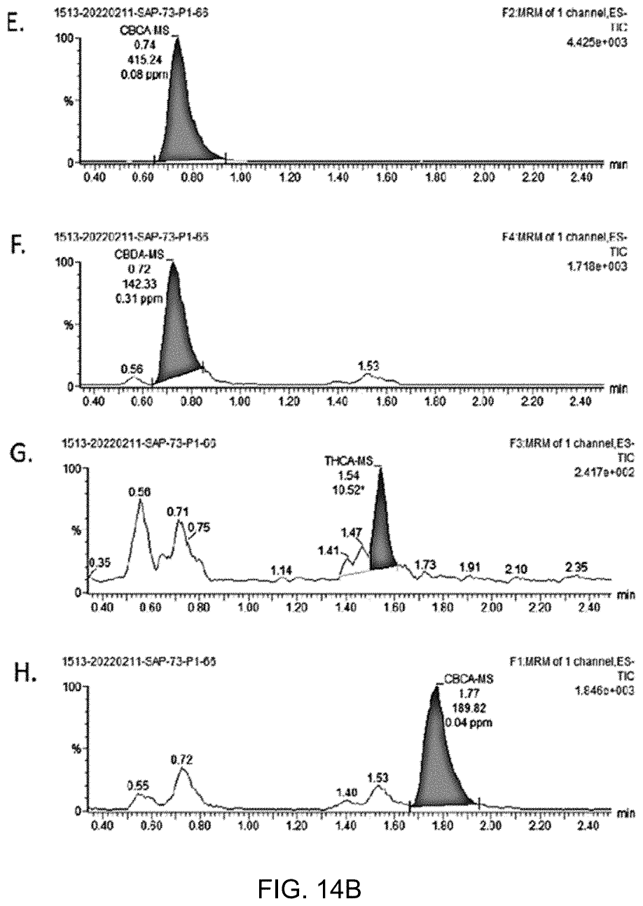

FIG. 14A and FIG. 14B show the results. FIG. 14A shows Panels A-D illustrating the production of meroterpenoids in HB3167 red fluorescent protein control (RFP). FIG. 14B shows Panels E-H illustrating red fluorescent protein control production of meroterpenoids in HB3167 transformed with OXC157. Integrated peaks (shaded solid peaks) indicate the presence of a specific Meroterpenoid. The integrated peaks (solid fill peaks) shown in Panel A (FIG. 14A) and Panels E-F (FIG. 14B) indicate the presence of a specific meroterpenoid.

Quantification of meroterpenoid production was also performed and it was shown that CBCa scaled with the production of CBDa.

Table 21 shows the quantified production of meroterpenoids (ppm) on the basis of strain.

TABLE 21
Quantified Production of Meroterpenoids (ppm) by Strain

| Strain | Plasmid | OXC # | CBGa | THCa | CBDa | CBCa |
|---|---|---|---|---|---|---|
| HB3167 | PLAS400 | RFP | 94.32 | 0.00 | 0.00 | 0.00 |
| HB3802 | PLAS415 | OXC52 | 44.15 | 0.00 | 10.58 | 0.00 |
| HB3803 | PLAS419 | OXC52 | 62.73 | 11.75 | 0.00 | 0.00 |
| HB3804 | PLAS679 | OXC157 | 0.89 | 2.56 | 46.14 | 12.24 |
| HB3805 | PLAS646 | OXC158 | 2.33 | 2.51 | 51.88 | 14.05 |

Table 22 lists characteristics of the strains utilized in this Example, beyond those strains already described in previous Examples 1 to 5.

TABLE 22
List of Strains Described in Examples 6

| Strain # | Plasmid | Genotype/base strain | Notes |
|---|---|---|---|
| HB3167 | none | A CBGa-producing Saccharomyces | Base strain for CBCa production |
| | | cerevisiae strain, similar to HB965 in | assays |
| | | example 1 | |
| HB3802 | PLAS-400 | Saccharomyces cerevisiae base strain | Gal1p: RFP: Cyc1t |
| | | HB3167 | |
| HB3803 | PLAS-415 | Saccharomyces cerevisiae base strain | VB40_OST1_pro-alpha- |
| | | HB3167 | f(I)_OXC52 |
| HB3804 | PLAS-419 | Saccharomyces cerevisiae base strain | VB40_ostl-proaf(I)_OXC53 |
| | | HB3167 | |
| HB3805 | PLAS-646 | Saccharomyces cerevisiae base strain | VB40_ost1-proaf1-OXC158 |
| | | HB3167 | (PLT1675-C8; OXC154-R3G(=GGG)/ |
| | | | A18E(=GAG)/S60T(=ACG)/ |
| | | | G351I(=ATC)/A383V(=GTG)/ |
| | | | L451G(=GGC)) |

Table 23 describes the plasmids used in this Example, beyond those already described in previous Examples 1 to 5.

TABLE 23
Plasmids

| | Plasmid | SEQ ID | | |
|---|---|---|---|---|
| # | Name | NO. | Description | Selection |
| 85 | PLAS400 | NO. 208 | Gal1p: RFP: Cyc1t | Uracil |
| 86 | PLAS419 | NO. 209 | VB40_ostl-proaf(I)_OXC53 | Uracil |
| 87 | PLAS646 | NO. 210 | VB40_ost1-proaf1-OXC158 | Uracil |
| | | | (PLT1675-C8; OXC154-R3G(=GGG)/ | |
| | | | A18E(=GAG)/S60T(=ACG)/ | |
| | | | G351I(=ATC)/A383V(=GTG)/ | |
| | | | L451G(=GGC)) | |

Table 24 lists certain sequences described in this Example, beyond those already described in previous Examples 1 to 5.

TABLE 24
Additional Sequences

| | | DNA/ | Length of | Position of coding |
|---|---|---|---|---|
| SEQ ID NO: | Description | Protein | sequence | sequence |
| SEQ ID NO. 208 | PLAS400 | DNA | 699 | 264-3347 |
| SEQ ID NO. 209 | PLAS419 | DNA | 1830 | 1-1830 |
| SEQ ID NO. 210 | PLAS646 | DNA | 1830 | 2649-478 |

Strains HB3804 and HB3805 with plasmids PLAS-419 and PLAS-646, respectively (based on Saccharomyces cerevisiae base strain HB3167) showed significant CBCa production. Noting HB3804 as having VB40_ostl-proaf(I)_OXC53 (SEQ ID NO: 209, DNA) and noting HB3805 as having VB40_ostl-proaf1-OXC158 (PLT1675-C8; OXC154-R3G(=GGG)/A18E(=GAG)/560T(=ACG)/G351 I(=ATC)/A383V(=GTG)/L451G(=GGC)) (SEQ ID NO: 210, DNA; SEQ ID N0:211, protein).

Noting for SEQ ID NO:209 and SEQ ID NO:210, relevant to the described sequences of length of 7266, the position of the coding sequence is at 2925 to 4499 encodes a protein of 524 residues with the noted modifications.

### Examples Only

In the preceding description, for purposes of explanation, numerous details are set forth in order to provide a thorough understanding of the embodiments. However, it will be apparent to one skilled in the art that these specific details are not required.

The embodiments described herein are intended to be examples only. Alterations, modifications and variations can be made to the particular embodiments by those of skill in the art. The scope of the claims should not be limited by the particular embodiments set forth herein, but should be construed in a manner consistent with the specification as a whole.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

### REFERENCES

All publications, patents and patent applications mentioned in this specification are indicative of the level of skill those skilled in the art to which this invention pertains and are herein incorporated by reference to the same extent as if each individual publication, patent, or patent application was specifically and individually indicated to be incorporated by reference.

### Patents and Applications

U.S. Pat. No. 7,361,482
U.S. Pat. No. 8,884,100 (Page et al.) Aromatic Prenyltransferase from Cannabis.
WO2018/148848 (Mookerjee et al.) publication of PCT/CA2018/050189, METHOD AND CELL LINE FOR PRODUCTION OF PHYTOCANNABINOIDS AND PHYTOCANNABINOID ANALOGUES IN YEAST
WO2018/148849 (Mookerjee et al.) publication of PCT/CA2018/050190, METHOD AND CELL LINE FOR PRODUCTION OF POLYKETIDES IN YEAST.
WO2020/232553 (Bourgeois et al.) publication of PCT/CA2020/050687, METHODS AND CELLS FOR PRODUCTION OF PHYTOCANNABINOIDS AND PHYTOCANNABINOID PRECURSORS.
WO2020/208411 (Szamecz et al.) publication of PCT/IB2020/000241, Microorganisms and methods for the fermentation of cannabinoids.

### Non-Patent Literature

Bai Flagfeldt, D., Siewers, V., Huang, L. and Nielsen, J. (2009) “Characterization of chromosomal integration sites for heterologous gene expression in Saccharomyces cerevisiae” Yeast, 26, 545-551.
Eisenberg, D., Schwarz, E., Komaromy, M., & Wall, R. (1984). “Analysis of membrane and surface protein sequences with the hydrophobic moment plot”. Journal of Molecular Biology, 179(1), 125-142. https://doi.org/10.1016/0022-2836(84)90309-7.
Gagne, S. J., et al. (2012) “Identification of Olivetolic Acid Cyclase from Cannabis Sativa Reveals a Unique Catalytic Route to Plant Polyketides.” Proceedings of the National Academy of Sciences, vol. 109, No: 31, 2012, pp. 12811-12816. doi:10.1073/pnas.1200330109.
Ghosh, R., A. Chhabra, P. A. Phatale, S. K. Samrat, J. Sharma, A. Gosain, D. Mohanty, S. Saran and R. S. Gokhale (2008) “Dissecting the Functional Role of Polyketide Synthases in Dictyostelium discoideum biosynthesis of the differentiation regulating factor 4-methyl-5-pentylbenzene-1,3-diol” Journal of Biological Chemistry, 283(17), 11348-11354.
Gietz, R. D., & Woods, R. A. (2006). Yeast transformation by the LiAc/SS Carrier DNA/PEG method. In Yeast Protocol (pp. 107-120). Humana Press, Totowa, N.J.
Gietz, R. D. and Schiestl, R. H., (2007) “High-efficiency yeast transformation using the LiAc/SS carrier DNA/PEG method.” Nat. Protoc. 2, 31-34.
Gietz R. D. (2014) Yeast Transformation by the LiAc/SS Carrier DNA/PEG Method (pp 1-12). In: Smith J., Burke D. (eds) Yeast Genetics. Methods in Molecular Biology (Methods and Protocols), vol 1205. Humana Press, New York, N.Y. https://doi.org/10.1007/978-1-4939-1363-3_1.
Jensen, N. B., Strucko, T., Kildegaard, K. R., David, F., et al., (2014). EasyClone: method for iterative chromosomal integration of multiple genes in Saccharomyces cerevisiae. FEMS Yeast Research, Volume 14, Issue 2, pages 238-248; https://doi.org/10.1111/1567-1364.12118.
Kim, J.-M., Song, H.-Y., Choi, H.-J., So, K.-K., Kim, D.-H., Chae, K.-S., . . . Jahng, K.-Y. (2015). “Characterization of NpgA, a 4′-phosphopantetheinyl transferase of Aspergillus nidulans, and evidence of its involvement in fungal growth and formation of conidia and cleistothecia for development.” Journal of Microbiology, 53(1), 21-31 https://doi.org/10.1007/s12275-015-4657-8.
Kuzuyama et al. (2005) Structural basis for the promiscuous biosynthetic prenylation of aromatic natural products, Nature, volume 435, pages 983-987; doi: 10.1038/nature03668.
Liu, J., Zhang, W., Du, G., Chen, J., & Zhou, J. (2013). “Overproduction of geraniol by enhanced precursor supply in Saccharomyces cerevisiae.” Journal of Biotechnology, 168(4), 446-451. https://doi.org/10.1016/J.JBIOTEC.2013.10.017.
Luo, X., Reiter, M., d'Espaux, L., Wong, J., Denby, C., Lechner, A., Zhang, Y., Grzybowski, A., Harth, S., Lin, W., Lee, H., Yu, C., Shin, J., Deng, K., Benites, V., Wang, G., Baidoo, E., Chen, Y., Dev, I., Petzold, C. and Keasling, J. (2019). “Complete biosynthesis of cannabinoids and their unnatural analogues in yeast.” Nature, 567(7746), pp. 123-126.
Oswald, Marilyne; Marc Fischer, Nicole Dirninger, Francis Karst, (2007) “Monoterpenoid biosynthesis in Saccharomyces cerevisiae.” FEMS Yeast Research, 7(3), 413-421. https://doi.org/10.1111/j.1567-1364.2006.00172.x
Peng, B., Nielsen, L. K., Kampranis, S. C., & Vickers, C. E. (2018). Engineered protein degradation of farnesyl pyrophosphate synthase is an effective regulatory mechanism to increase monoterpene production in Saccharomyces cerevisiae. Metabolic Engineering, 47, 83-93. https://doi.org/10.1016/J.YMBEN.2018.02.005.
Peng, B., Wood, R. J., Nielsen, L. K., Vickers, C. E. (2018). An Expanded Heterologous GAL Promoter Collection for Diauxie-Inducible Expression in Saccharomyces cerevisiae. ACS Synth Biol. 7(2):748-751. https://doi.org/10.1021/acssynbio.7b00355
Reider Apel A, d'Espaux L, Wehrs M, et al. A Cas9-based toolkit to program gene expression in Saccharomyces cerevisiae. Nucleic Acids Res. 2017; 45(1):496-508. doi:10.1093/nar/gkw1023
Ro, D.-K., Paradise, E. M., Ouellet, M., Fisher, K. J., Newman, K. L., Ndungu, J. M., Keasling, J. D. (2006). Production of the antimalarial drug precursor artemisinic acid in engineered yeast. Nature, 440(7086), 940-943. JOUR. https://doi.org/10.1038/nature04640.
Ryan, O. W., Poddar, S., & Cate, J. H. D. (2016). CRISPR-Cas9 Genome Engineering in Saccharomyces cerevisiae Cells. Cold Spring Harbor Protocols, 2016(6), pdb.prot086827. https://doi.org/10.1101/pdb.prot086827.
Saeki, H., Hara, R., Takahashi, H., Iijima, M., Munakata, R., Kenmoku, H., . . . Taura, F. (2018). An Aromatic Farnesyltransferase Functions in Biosynthesis of the Anti-HIV Meroterpenoid Daurichromenic Acid. Plant Physiology, 178(2), 535-551; https://doi.org/10.1104/PP.18.00655.
Shi, S., Chen, Y., Siewers, V., & Nielsen, J. (2014). “Improving Production of Malonyl Coenzyme A-Derived Metabolites by Abolishing Snf1-Dependent Regulation of Acc1.” mBio, 5(3), e01130-14. https://doi.org/10.1128/mBio.01130-14.
Shiba, Y., Paradise, E. M., Kirby, J., Ro, D.-K., & Keasling, J. D. (2007). “Engineering of the pyruvate dehydrogenase bypass in Saccharomyces cerevisiae for high-level production of isoprenoids.” Metabolic Engineering, 9(2), 160-168. https://doi.org/10.1016/J.YMBEN.2006.10.005.
Sirikantaramas, S., Taura, F., Tanaka, Y., Ishikawa, Y., Morimoto, S., & Shoyama, Y. (2005). “Tetrahydrocannabinolic acid synthase, the enzyme controlling marijuana psychoactivity, is secreted into the storage cavity of the glandular trichomes”. Plant and Cell Physiology, 46(9), 1578-1582.
Stout, J. M., Boubakir, Z., Ambrose, S. J., Purves, R. W., & Page, J. E. (2012). The hexanoyl-CoA precursor for cannabinoid biosynthesis is formed by an acyl-activating enzyme in Cannabis sativa trichomes. The Plant Journal, 71(3), 353-365.
Taura, F., Morimoto, S., & Shoyama, Y. (1996). Purification and characterization of cannabidiolic-acid synthase from Cannabis sativa L. Biochemical analysis of a novel enzyme that catalyzes the oxidocyclization of cannabigerolic acid to cannabidiolic acid. Journal of Biological Chemistry, 271(29), 17411-17416.
van Hoek, Pim, et al. “Fermentative capacity in high-cell-density fed-batch cultures of baker's yeast.” Biotechnology and bioengineering 68.5 (2000): 517-523.
Varshavsky, A. (1996). The N-end rule: functions, mysteries, uses. Proceedings of the National Academy of Sciences, 93(22), 12142-12149.
Varshavsky, A. (2011). The N-end rule pathway and regulation by proteolysis. Protein Science 20(8):1285-1476. https://doi.org/10.1002/pro.666.
Wu, X-L., Li, B-Z., Zhang, W-Z., Kong, K., Qi, H., Dai, J-b., & Yuan, Y-J. (2017). Genome-Wide Landscape of Position Effects on Heterogeneous Gene Expression in Saccharomyces cerevisiae. Biotechnology for Biofuels. 10(189) https://doi.org/10.1186/s13068-017-0872-3.
Zamberletti, E., Rubino, T., & Parolaro, D. (2021). Therapeutic potential of cannabidivarin for epilepsy and autism spectrum disorder. Pharmacology & Therapeutics, 107878.

1. A method of producing cannabidiolic acid (CBDa) or a phytocannabinoid produced therefrom in a heterologous host cell comprising CBDa-producing, CBCa-producing, or other phytocannabinoid-producing capacity, said method comprising:
transforming said host cell with a nucleotide encoding a variant CBDa synthase protein having a serine insertion between residues P224 and K225 and one or more other amino acid mutations relative to the wild type CBDa synthase protein OXC52 (SEQ ID NO:140), and
culturing said transformed host cell to produce CBDa, CBCa, and/or a phytocannabinoid therefrom,
wherein said variant CBDa synthase protein comprises at least 85%, 90%, 95%, or 99% sequence identity with OXC154 (SEQ ID NO:141).

2. The method of claim 1, wherein the one or more amino acid mutations is at a location:
selected from the group consisting of residues 451, 2, 3, 5, 18, 21, 26, 28, 31, 47, 49, 60, 88, 97, 225, 274, 295, 331, 347, 349, 351, 367, 372, 383, 399, 513, and 515 of OXC154 (SEQ ID NO:141),
selected from the group consisting of residues 451, 3, 18, 49, and 97 of OXC154, or
selected from the group consisting of residues 451, 3, and 18 of OXC154.

3. The method of claim 1, wherein said variant CBDa synthase protein has a non-conservative amino acid substitution in at least 2 amino acid locations
selected from the group consisting of: residues 451, 2, 3, 5, 18, 21, 26, 28, 31, 47, 49, 60, 88, 97, 225, 274, 295, 331, 347, 349, 351, 367, 372, 383, 399, 513, and 515 of OXC154 (SEQ ID NO:141),
selected from the group consisting of residues 451, 3, 18, 49, and 97 of OXC154, or
selected from the group consisting of residues 451, 3, and 18 of OXC154.

4. The method of claim 1, wherein said variant CBDa synthase protein comprises:
an amino acid mutation at 451, and
at least one other mutation comprising a non-conservative amino acid substitution at a location
selected from the group consisting of: residues 2, 3, 5, 18, 21, 26, 28, 31, 47, 49, 60, 88, 97, 225, 274, 295, 331, 347, 349, 351, 367, 372, 383, 399, 513, and 515 of OXC154 (SEQ ID NO:141),
selected from the group consisting of residues 3, 18, 49, and 97 of OXC154, or
selected from the group consisting of residues 3 and 18 of OXC154.

5. The method according to claim 1, wherein the nucleotide encoding the variant CBDa synthase protein has a sequence comprising:
(a) a nucleotide sequence according to:
SEQ ID NO:187,
SEQ ID NO:4-71,
SEQ ID NO:157-160,
SEQ ID NO:165-172,
SEQ ID NO:181-186, 188, 209, or 210;
(b) a nucleotide sequence having at least 85%, at least 90%, at least 95%, or at least 99%, identity with the sequence of (a); or
(c) a nucleotide sequence that hybridizes with the complementary strand of the nucleotide having the sequence of (a).

6. The method according to claim 1, wherein the variant CBDa synthase protein comprises:
(a) a sequence according to:
SEQ ID NO:195,
SEQ ID NO:72-139,
SEQ ID NO:161-164,
SEQ ID NO:173-180,
SEQ ID NO:189-194, 196, or 211;
(b) a sequence of at least 85%, at least 90%, at least 95%, or at least 99%, identity with the sequence of (a).

7. The method according to claim 1, wherein the amino acid mutations relative to OXC154 (SEQ ID NO:141), are selected from the group consisting of:
L451G;
P2W;
R3G, R3T, R3W, R3V, or R3A;
N5Q;
A18E;
L21G;
T26A;
N28E;
L31E;
S47F;
T49R;
S60T;
S88A;
V97E or V97D;
Q274G;
N331G;
A347G;
Q349G;
G351I, G351R, or G351M;
S367Q; S367N; S367R; or S367K;
1372L;
A383V;
V383A; V383M; V383G;
S399G;
L451G;
P513V; and/or
H515E.

8. The method of claim 1, wherein the host cell is transformed with a nucleotide encoding:
(a) a variant CBDa synthase protein with at least 85%, at least 90%, at least 95%, at least 99%, or 100% sequence identity of any one of the following sequences with the indicated substitutions from OXC154 (SEQ ID NO:141):
OXC154-S88A/L451G (SEQ ID NO:72),
OXC154-R3G/L21G/S60T/S88A (SEQ ID NO:73),
OXC154-R3G/A18E/T49R/S60T/S88A (SEQ ID NO:74),
OXC154-R3T/T49R/S88A (SEQ ID NO:75),
OXC154-R3W/A18E/T49R/S60T/S88A (SEQ ID NO:76),
OXC154-R3V/T49R/S60T/S88A (=GCT) (SEQ ID NO:77),
OXC154-R3V/T49R/S60T/S88A (=GCC) (SEQ ID NO:78),
OXC154-A18A (SEQ ID NO:79),
OXC154-R3T/A18E/T49R/S88A (=GCC) (SEQ ID NO:80),
OXC154-R3T/S88A (=GCC) (SEQ ID NO:81),
OXC154-R3G(=GGG)/L21G/T49R (=GCC) (SEQ ID NO:82),
OXC154-R3T/T49R/S88A(=GCT) (SEQ ID NO:83),
OXC154-R3G(=GGA)/A18E/T49R/S60T/S88A(=GCC) (SEQ ID NO:84),
OXC154-R3W/T49R/S88A(=GCC)/V97E (SEQ ID NO:85),
OXC154-R3G(=GGG)/A18E/S88A(=GCC) (SEQ ID NO:86),
OXC154-R3V/A18E/T49R/S60T/S88A(=GCC) (SEQ ID NO:87),
OXC154-S60T/S88A(=GCC) (SEQ ID NO:88),
OXC154-R3T/A18E/T49R/S60T/S88A(=GCT) (SEQ ID NO:89),
OXC154-R3W/L21G/T49R/S88A(=GCC)/V97E (SEQ ID NO:90),
OXC154-R3T/A18E/T49R/S60T (SEQ ID NO:91),
OXC154-P2W/T26A/S60T (SEQ ID NO:91),
OXC154-R3G(=GGG)/L21G/S60T/S88A(=GCC)/V97E (SEQ ID NO:93),
OXC154-R3G(=GGG)/A18E/T49R/S88A(=GCC) (SEQ ID NO:94),
OXC154-R3T/L21G/S60T/S88A(=GCC)/V97D (SEQ ID NO:95),
OXC154-P2W/L21G/T49R/S88A(=GCC)/V97E (SEQ ID NO:96),
OXC154-R3G(=GGG)/L21G/T49R/S88A(=GCT) (SEQ ID NO:97),
OXC154-S295S(=TCA) (SEQ ID NO:98),
OXC154-R3V/L21G/S60T/S88A(=GCC) (SEQ ID NO:99),
OXC154-R3T/A18E/S88A(=GCC) (SEQ ID NO:100),
OXC154-S60T/S88A(=GCT) (SEQ ID NO:101),
OXC154-R3W/T49R/S88A(=GCT) (SEQ ID NO:102),
OXC154-T49R/S88A(=GCC) (SEQ ID NO:103),
OXC154-R3W/S47F (SEQ ID NO:104),
OXC154-A347G/I372L/L451G (SEQ ID NO:105),
OXC154-R3G(=GGG)/L21G/S60T (SEQ ID NO:106),
OXC154-R3T/L21G/T49R/S88A(=GCT) (SEQ ID NO:107),
OXC154-R3T/L21G/S60T (SEQ ID NO:108),
OXC154-R3W/L21G/S88A(=GCT) (SEQ ID NO:109),
OXC154-L21G/T49R/S60T/S88A(=GCT) (SEQ ID NO:110),
OXC154-A347G/A383V (SEQ ID NO:111),
OXC154-R3W/L21G/T49R/S60T/S88A(=GCT) (SEQ ID NO:112),
OXC154-A18E/S88A(=GCC) (SEQ ID NO:113),
OXC154-R3W/L21G/T49R (SEQ ID NO:114),
OXC154-A347G/L451G (SEQ ID NO:115),
OXC154-A347G/I372L/A383V/L451G (SEQ ID NO:116),
OXC154-I372L/A383V/L451G (SEQ ID NO:117),
OXC154-R3V/T49R/S88A(=GCT) (SEQ ID NO:118),
OXC154-R3G(=GGG)/A18E/S60T (SEQ ID NO:119),
OXC154-A347G/I372L/A383V (SEQ ID NO:120),
OXC154-R3T (SEQ ID NO:121),
OXC154-R3V/A18E/T49R/V97E (SEQ ID NO:122),
OXC154-R3T/L21G/T49R/S60T/S88A(=GCT) (SEQ ID NO:123),
OXC154-R3T/L21G/T49R/V97E (SEQ ID NO:124),
OXC154-R3V/L21G/T49R/S60T (SEQ ID NO:125),
OXC154-G351I/1372L (SEQ ID NO:126),
OXC154-G351I/A383V/L451G (SEQ ID NO:127),
OXC154-G351R/I372L/L451G (SEQ ID NO:128),
OXC154-G351I/I372L/A383V/L451G (SEQ ID NO:129),
OXC154-G351R/I372L/A383V/L451G (SEQ ID NO:130),
OXC154-G351I/I372L/A383V (SEQ ID NO:131),
OXC154-N331G/Q349G/I372L/L451G (SEQ ID NO:132),
OXC154-G351R/A383V/L451G (SEQ ID NO:133),
OXC154-Q349G/A383V/L451G (SEQ ID NO:134),
OXC154-A383V/L451G (SEQ ID NO:135),
OXC154-N331G/Q349G (SEQ ID NO:136),
OXC154-G351I (SEQ ID NO:137),
OXC154-L451G (SEQ ID NO:138),
OXC154-N331G/G351I/I372L/A383V (SEQ ID NO:139),
OXC154-R3G/A18E/S60T/G351I/A383V/L451G (SEQ ID NO:161),
OXC154-R3W/A18E/T49R/V97E/G351I/A383V/L451G (SEQ ID NO:162),
OXC154-R3W/A18E/T49R/V97E/G351I/A383V/L451G (SEQ ID NO:163),
OXC154-R3T/S60T/G351I/A383V/L451G (SEQ ID NO:164),
OXC154-R3G/A18E/S60T/G351I/A383V/L451G (SEQ ID NO: 211);
or
(b) a variant CBDa synthase protein with at least 85%, at least 90%, at least 95%, at least 99% sequence identity, or with 100% identity with any one of the following sequences with the further indicated substitutions from OXC158 (SEQ ID NO:162):
OXC158-W3A/I351G/V383A (SEQ ID NO:195),
OXC158-I351G (SEQ ID NO:173),
OXC158-S367R(=CGG) (SEQ ID NO:174),
OXC158-Q274G (SEQ ID NO:175),
OXC158-I351M (SEQ ID NO:176),
OXC158-V383A (SEQ ID NO:177),
OXC158-S367Q (SEQ ID NO:178),
OXC158-S367N (SEQ ID NO:179),
OXC158-S367R(=AGG) (SEQ ID NO:180),
OXC158-L31E/V383G (SEQ ID NO:189),
OXC158-N138T/V383M/H515E (SEQ ID NO:190),
OXC158-S367K/V383A/P513V (SEQ ID NO:191),
OXC158-V383A (SEQ ID NO:192),
OXC158-W3A/L31E/K226M/S367Q/V383M/S399G/P513V (SEQ ID NO:193),
OXC158-I351GN383A (SEQ ID NO:194), or
OXC158-W3A/N5Q/N28E/I351G/S367R/V383A (SEQ ID NO:196).

9. The method of claim 1, wherein said phytocannabinoid produced is cannabigerol (CBG), cannabigerolic acid (CBGa), cannabigerovarin (CBGv), cannabigerovarinic acid (CBGVa), cannabigerocin (CBGO), cannabigerocinic acid (CBGOa), cannabidiovarinic acid (CBDVa), cannabichromenic acid (CBCa), cannabichromene (CBC), tetrahydrocannabinol (THC), or tetrahydrocannabinolic acid (THCa).

10. The method of claim 9, wherein the transformed host cell produces cannabidiovarinic acid (CBDVa) from cannabigerovarinic acid (CBGVa), optionally in the presence of endogenously produced or exogenously provided butyric acid.

11. The method of claim 1, wherein said host cell is a yeast cell, a bacterial cell, a fungal cell, a protist cell, or a plant cell.

12. The method of claim 11, wherein said host cell is S. cerevisiae, E. coli, Yarrowia lipolytica, or Komagataella phaffii.

13. The method of claim 1, wherein said transformed host cell additionally comprises a polynucleotide encoding a polyketide synthase enzyme, a polynucleotide encoding an olivetolic acid cyclase enzyme, and/or a polynucleotide encoding a prenyltransferase enzyme.

14. The method of claim 1, wherein said transformed host cell additionally comprises a polynucleotide encoding a type III PKS, an acyl-activating enzyme, a prenyltransferase enzyme, and/or an oxidocyclase enzyme.

15. An isolated polypeptide having cannabidiolic acid synthase activity comprising an amino acid sequence of at least 85%, of at least 90%, of at least 95%, of at least 99%, or of 100% sequence identity relative to OXC154 (SEQ ID NO:141), wherein one or more amino acid residues comprise mutations relative to OXC154 (SEQ ID NO:141), at least one of said one or more mutation being located at a position selected from the group consisting of: residues 451, 2, 3, 5, 18, 21, 26, 28, 31, 47, 49, 60, 88, 97, 225, 274, 295, 331, 347, 349, 351, 367, 372, 383, 399, 513, and 515 of SEQ ID NO:141.

16. The isolated polypeptide of claim 15, comprising an amino acid sequence having at least at least 85%, at least 90%, at least 95%, at least 99%, or 100% sequence identity with:
SEQ ID NO:195,
SEQ ID NO:72-139,
SEQ ID NO:161-164,
SEQ ID NO:173-180,
SEQ ID NO:189-194, 196, or 211.

17. An isolated polynucleotide encoding a polypeptide having cannabidiolic acid synthase activity comprising:
(a) a nucleotide sequence according to:
SEQ ID NO:187,
SEQ ID NO:4-71,
SEQ ID NO:157-160,
SEQ ID NO:165-172,
SEQ ID NO:181-186, 188, 209, or 210;
(b) a nucleotide sequence having at least 85%, at least 90%, at least 95%, or at least 99% sequence identity with the nucleotide sequence of (a); or
(c) a nucleotide sequence that hybridizes with the complementary strand of the nucleotide having the sequence of (a).

18. An expression vector comprising the polynucleotide according to claim 17, encoding a protein having CBDa synthase activity.

19. The expression vector of claim 18, wherein the polynucleotide encoding the polypeptide having CBDa synthase activity comprises the nucleotide sequence according to:
SEQ ID NO:187,
SEQ ID NO:4-71,
SEQ ID NO:157-160,
SEQ ID NO:165-172,
SEQ ID NO:181-186, 188, 209 or 210.

20. A host cell transformed with the expression vector of claim 18.

21. The host cell of claim 20, additionally comprising a polynucleotide encoding a polyketide synthase enzyme, a polynucleotide encoding an olivetolic acid cyclase enzyme, and/or a polynucleotide encoding a prenyltransferase enzyme.

22. The host cell of claim 20, additionally comprises a polynucleotide encoding a type III PKS, an acyl-activating enzyme, a prenyltransferase enzyme, and/or an oxidocyclase enzyme.
